SEQUENCE LISTING (35 sequences)

SEQ ID NO: 1 - DNA, 3619 bp, Homo sapiens
aataggaaaaaaaggaagcaaattacttctgggtaatgtttagaaattccctaacagatggtatttttctcttgaatagatcaaaacgtttcagataaatcatatttctatgaaacaaggataagattcgtctgttacgtatgaaaaaaactaatgcaggattgagtttacgttaagtattgagacccagaactctgcctcttgggcaaagccaagcataaagcagcaagccctcccagaattagacggctttccacggacaggagaacagatacagcagacacagaatgactacagaaggcgaaaacaagcaaaaacaaatgtcactccatctggttttaagccaaataagccctccaaccctaaccaaggcaaaggagaactgcctttccacagaacacacctgactggtttcagtacttgttttcccacgagtacccttgaaactcattttaatccaaaaatgaaaactatgggacaagcacggtggctcatgcctgtaatcccagcacttgtgggaggctgaggcgggcggatcatctgaggtcaggagtttgagaccagcctgaccgatatggcgaaaccacatctttactaaatacaaaaaattagctgggcgaggtagcaggcgcctgtaatcccagctactagggaggctgaggcaggagaatcgcatgaacccaggaggcggaggttgcagtgagtcgagattgcgccattgcactccagctagggcaacaagagcaaaacttcgtctcaaaaacaaacaaacaaaaactagtattttgtgatacagccagaactatcacaaggaaaagactcccagtactgtatcataatacttccacataaaatacctcctttagggatggggtcatttctcccgggataattcaattcaatgcttaatttttgagaagggctgcatgaaggagagacttgtagttaaaaaaaaaaaacgggtctaatttctggttctggtacttactagctgggttaccttatgcgagaattcttaatttcctagtcttagtttcctcaccttcaaattgagactaataactactttgtaggtctctggtaagagggagagagaaactaagcggttggaacaccatgggcactcaataatagctatcatttacataatgagtccctactaaatatggatgctgggttaggcgctgccgagatacaaggtgaaagttctagctgcaaatggcaaaagtcctgaatgttcttggatttcaaaatgctttttgtatatcgcctcttttctgcccgtgaggaggacaagggcaaacaattaaaataggaaaaatgtggggagcacttcatggtttataaaacgtttttctgcgtgccaacttattttaatgtcttaactttccagatttgaaaacaagtcctacaccaaataaatggtagagggacacaaactggaacccaggcttcctaaccgccagagcgaccttcccgccgcccaatgcctccctgaggtgctggtcggctgcttctgcccggggtacccagcagtcatcctactctccactcagcaccaccgcacgtcggccgcgcgccacgctacccgccgggttactgggcgctggtttggccttcggaggtcgaggctctctgagtgacctcctgaactgggctcactcttacttagcctcctctctcgaggcagcaccctcctctctacacgacaactccatcattcgcagccccacggtgttcccggggagcgcctcccgcccccaccggcttcgcctcagcttgtcctcgcctttcccgccctcagcatccgcggatacccgcgctcgccgggcccacgcggaagcatccgcagactgtcgcaaggtcccaccgtcttgctctcattgggcaaccttaccctttccccgcctcaatgtttttgcttcctcagtgattggacctgggcattgtccattccagctcccaggcaccgccctctctccacccactagaaagccacccgggttctccttcatccccaacgctcctcacctagcccctgtcctgagggtgcccctaaccctggagccagctttaaaggggcaggccccttctcctgccccagccccagccccacccaccgggcccacattctccgtcttccaggtccagcgctccctcacagaactcccccgcaccttgtctcgctcgctccgcccctccttcccagccacagctagaggtcccgggcgcacctgcagagccggaggcttgctggggcatgcgctccatgaaggctctgcagaaggccctgagccgggctggcagtcactgcgggcgaggaggctggggtcacccgagccggagccccctccttggcgggggcgtccggcaccacctcagtgaggccgcggcgcagggcagagagacgccacacagccaccagccgcagcaccaggatcagtaaggctcctgggacagggcaggggacgggcttgcccccactcccactgtggttctcgctagttcccgagtgcccttccaaggcagcgccgctcatctcccctctcctcgctcactagccctcggtgccttttcctttggatgtggggctttgggtgcgccgcgctcgggcggtaatctgccgtctgcctaggagtgcgttcgggcaagcgggtcctggggatggcgcctgtccaaggtgcgcacttaacccggatcacagatgcgcgccgagtgttgcttggctcacgtgtctgagtgcagggccgggctcacgccgccagtcctcaccttccctagtcctcgtgtgtattttaggagatgcgtggatgtggaacagcctcctgcctccggtccaggtgtactggggtctgtgtgttgtgtttctgcgtgttctcggcaggtaatgtgtgatgcgcgtgtattattggtgcgcaggtacgtccataagtgttgggtgtgtgcgtagcagtgctccctgaacagcagaatgggggggaatttccaaactggttgttgaatggggagaatatttgtaatggaggaaaggtctttaagacgcagatttcccaaaccagagccagtcagagcctttatttgtgctgcccaggctccagaatctaaaacgttctcttcctttgggtgtttcttccacccaggatgccaaacctttatcttccccaccgcctaggacacacttggtggctgagcctttttctctgagacccagacggtgctgagcaggaaatcttgtcctccccagagggccagcatagacactgtccaaagagcccctgctctactgtagcaggaacaggaaatgggccatctccctcgtcgctgccatctccctgatgacactcagaaacatggacaccatcagtaaagtagccagactgacacaacactacctcaccactactcgagtctctgacacaacactacctcaccactactcgagtctctgtcgtaagggacaccaataggtattatttattattaatgacattatgcagtacttttatttctttcttttttgagacggagtctcactctgtcacacaggctggagtgcagtggcaccatctcgactcactgcaatctccacctcctgggttcaagtaattctcctgcctca

SEQ ID NO: 2 - DNA, 12 bp, Artificial Sequence
  consensus motif 1
cgagatagcgcc

SEQ ID NO: 3 - DNA, 12 bp, Artificial Sequence
  consensus motif 2
cgagatagtgcc

SEQ ID NO: 4 - DNA, 20 bp, Artificial Sequence
  Primer 18S rRNA FWD
cggcgacgacccattcgaac

SEQ ID NO: 5 - DNA, 23 bp, Artificial Sequence
  Primer 18S rRNA REV
gaatcgaaccctgattccccgtc

SEQ ID NO: 6 - DNA, 21 bp, Artificial Sequence
  Primer GLS FWD
tctacaggattgcgaacgtct

SEQ ID NO: 7 - DNA, 23 bp, Artificial Sequence
  Primer GLS REV
ctttgtctagcatgacaccatct

SEQ ID NO: 8 - DNA, 21 bp, Artificial Sequence
  Primer GLS2 FWD
gcctgggtgatttgctctttt

SEQ ID NO: 9 - DNA, 22 bp, Artificial Sequence
  Primer GLS2 REV
cctttagtgcagtggtgaactt

SEQ ID NO: 10 - DNA, 23 bp, Artificial Sequence
  Primer SLC7A11 FWD
ggtccattaccagcttttgtacg

SEQ ID NO: 11 - DNA, 21 bp, Artificial Sequence
  Primer SLC7A11 REV
aatgtagcgtccaaatgccag

SEQ ID NO: 12 - DNA, 35 bp, Artificial Sequence
  Primer GLS2_000_10F
aggaagagagggaggtaggaggttgttttatattt

SEQ ID NO: 13 - DNA, 56 bp, Artificial Sequence
  Primer GLS2_000_T7R
cagtaatacgactcactatagggagaaggctcttacccccactcccactataattc

SEQ ID NO: 14 - DNA, 34 bp, Artificial Sequence
  Primer GLS2_001_10F
aggaagagagattatagtgggagtgggggtaagt

SEQ ID NO: 15 - DNA, 56 bp, Artificial Sequence
  Primer GLS2_001_T7R
cagtaatacgactcactatagggagaaggcttcctaaaaatacccctaaccctaaa

SEQ ID NO: 16 - DNA, 35 bp, Artificial Sequence
  Primer GLS2_002_10F
aggaagagaggggtttgtttttttaaagttggttt

SEQ ID NO: 17 - DNA, 57 bp, Artificial Sequence
  Primer GLS2_002_T7R
cagtaatacgactcactatagggagaaggctactctcattaaacaaccttacccttt

SEQ ID NO: 18 - DNA, 36 bp, Artificial Sequence
  Primer GLS2_003_10F
aggaagagaggagttggaatggataatgtttaggtt

SEQ ID NO: 19 - DNA, 57 bp, Artificial Sequence
  Primer GLS2_003_T7R
cagtaatacgactcactatagggagaaggctcccaatacctccctaaaatactaatc

SEQ ID NO: 20 - DNA, 36 bp, Artificial Sequence
  Primer GLS2_004_10F
aggaagagagaaagggtaaggttgtttaatgagagt

SEQ ID NO: 21 - DNA, 56 bp, Artificial Sequence
  Primer GLS2_004_T7R
cagtaatacgactcactatagggagaaggctcacaaactaaaacccaaacttccta

SEQ ID NO: 22 - DNA, 33 bp, Artificial Sequence
  Primer GLS2_005_10F
aggaagagagtaaggtttttgggatagggtagg

SEQ ID NO: 23 - DNA, 55 bp, Artificial Sequence
  Primer GLS2_005_T7R
cagtaatacgactcactatagggagaaggctacacaaaccccaatacacctaaac

SEQ ID NO: 24 - DNA, 35 bp, Artificial Sequence
  Primer GLS2_006_10F
aggaagagagtgaaggttttgtagaaggttttgag

SEQ ID NO: 25 - DNA, 56 bp, Artificial Sequence
  Primer GLS2_006_T7R
cagtaatacgactcactatagggagaaggctccacaataaaaataaaaacaaaccc

SEQ ID NO: 26 - DNA, 35 bp, Artificial Sequence
  Primer GLS2_007_10F
aggaagagagtgattggatttgggtattgtttatt

SEQ ID NO: 27 - DNA, 56 bp, Artificial Sequence
  Primer GLS2_007_T7R
cagtaatacgactcactatagggagaaggctctcaaaaccttctacaaaaccttca

SEQ ID NO: 28 - DNA, 35 bp, Artificial Sequence
  Primer GLS2_008_10F
aggaagagaggtttaatgtttttttgaggtgttgg

SEQ ID NO: 29 - DNA, 57 bp, Artificial Sequence
  Primer GLS2_008_T7R
cagtaatacgactcactatagggagaaggctaaaataaacaatacccaaatccaatc

SEQ ID NO: 30 - DNA, 39 bp, Artificial Sequence
  Primer GLS2_009_10F
aggaagagagtaaataaatggtagagggatataaattgg

SEQ ID NO: 31 - DNA, 56 bp, Artificial Sequence
  Primer GLS2_009_T7R
cagtaatacgactcactatagggagaaggctttcaaaaaatcactcaaaaaacctc

SEQ ID NO: 32 - DNA, 21 bp, Artificial Sequence
  GLS shRNA 1
gcacagacatggttggtatat

SEQ ID NO: 33 - DNA, 21 bp, Artificial Sequence
  GLS shRNA 2
gcacagacatggttggtatat

SEQ ID NO: 34 - DNA, 21 bp, Artificial Sequence
  GLS2 shRNA 1
gccatggatatggaacagaaa

SEQ ID NO: 35 - DNA, 21 bp, Artificial Sequence
  GLS2 shRNA 2
gccctgtccaaagagaactta

(19) United States
(12) Patent Application Publication
(10) Pub. No.: US 2025/0275964 A1
(43) Pub. Date: Sep. 4, 2025

(54) METHODS OF INHIBITING LIVER-TYPE GLUTAMINASE, GLS2

(71) Applicant: CORNELL UNIVERSITY, Rochester, NY (US)

(72) Inventors: Richard CERIONE, Ithaca, NY (US); William P. KATT, Brooktondale, NY (US); Michael J. LUKEY, Ithaca, NY (US); Sekar RAMACHANDRAN, Ithaca, NY (US)

(21) Appl. No.: 17/769,307

(22) PCT Filed: Oct. 15, 2020
(86) PCT No.: PCT/US2020/055855

§ 371 Date: Apr. 14, 2022

Related U.S. Application Data
(60) Provisional application No. 62/915,414, filed on Oct. 15, 2019.

(51) Int. Cl. A61K 31/501; A61P 35/00

(52) U.S. Cl. CPC A61K 31/501; A61P 35/00; C12Y 305/01002

(57) ABSTRACT

The present application relates to a method of reducing the production of glutamate from glutamine by GLS and by GLS2 in a cancerous cell or cancerous tissue. This method includes inhibiting glutaminase activity of GLS and GLS2 in the cancerous cell or cancerous tissue by a method involving selecting a cancerous cell or cancerous tissue; and contacting GLS and GLS2 in the cell or tissue with a dual GLS/GLS2 inhibitor, where the contacting reduces the production of glutamate from glutamine by GLS and by GLS2 in the cell or tissue.

This application is a national stage application under 35 U.S.C. § 371 of International Application No. PCT/US2020/055855, filed Oct. 15, 2020, which claims the priority benefit of U.S. Provisional Patent Application Ser. No. 62/915,414, filed Oct. 15, 2019, which is hereby incorporated by reference in its entirety.

This invention was made with government support under grant numbers GM122575 and CA201402 awarded by the National Institutes of Health. The government has certain rights in this invention.

### SEQUENCE LISTING

This application contains a computer readable Sequence Listing, which has been submitted electronically in ASCII format (“Sequence Listing ASCII”) and is hereby incorporated by reference in its entirety. The Sequence Listing ASCII, created on Mar. 19, 2024, is named 147402.008602_ST25.txt and is 11,622 bytes in size.

### FIELD

The present application relates to a method of reducing the production of glutamate from glutamine by GLS and by GLS2 in a cancerous cell or cancerous tissue using a dual GLS/GLS2 inhibitor.

### BACKGROUND

Sustained biomass accumulation in tumors depends on cancer cells acquiring nutrients from the environment and processing them to meet the biosynthetic, bioenergetic, and redox demands of proliferation (Pavlova et al., “The Emerging Hallmarks of Cancer Metabolism,” Cell Metab. 23:27-47 (2016)). Many oncogenic signaling pathways regulate the expression, activity, or localization of nutrient transporters and metabolic enzymes, and extrinsic factors such as O₂ availability also influence cellular metabolism (Vander Heiden and DeBerardinis, “Understanding the Intersections Between Metabolism and Cancer Biology,” Cell 168:657-669 (2017)). These variables cause cancer cell metabolism to be highly heterogeneous in nature, although certain metabolic alterations are consistently observed in diverse tumor types. For example, most tumors exhibit elevated glucose uptake coupled to lactate secretion regardless of O₂ availability (the Warburg effect), and cancer cells also frequently depend on an exogenous supply of glutamine (Pavlova et al., “The Emerging Hallmarks of Cancer Metabolism,” Cell Metab. 23:27-47 (2016)).

Glutamine is the most abundant amino acid in blood serum and is a major source of carbon and nitrogen for tumor cells. Its uptake into cells is facilitated by plasma membrane transporters, which in some cases are essential for tumorigenesis (Van Geldermalsen et al., “ASCT2/SLC1A5 Controls Glutamine Uptake and Tumour Growth in Triple-Negative Basal-Like Breast Cancer,” Oncogene 35:3201-3208 (2016)). Once in the cytosol, there are several possible fates for glutamine in addition to its role as a proteinogenic amino acid. In mitochondria, glutamine catabolism is initiated by glutaminase, which releases the amide nitrogen as ammonia to generate glutamate. In turn, glutamate can be incorporated into the glutathione and proline biosynthesis pathways, or deaminated to produce the tricarboxylic acid (TCA) cycle intermediate α-ketoglutarate (α-KG). This metabolic pathway is widely upregulated in cancer cells, with glutamine serving as a key anaplerotic substrate for the TCA cycle (Cluntun et al., “Glutamine Metabolism in Cancer: Understanding the Heterogeneity,” Trends in Cancer 3:169-180 (2017)).

Two genes encode glutaminases in mammals, GLS and GLS2, and different isoforms of each enzyme arise from alternative splicing and surrogate promoter mechanisms (Katt et al., “A Tale of Two Glutaminases: Homologous Enzymes with Distinct Roles in Tumorigenesis,” Future Med. Chem. 9:223-243 (2017)). The GLS isozyme is ubiquitous in healthy tissues, whereas liver-type glutaminase (GLS2) is restricted primarily to the liver, pancreas, and brain (Altman et al., “From Krebs to Clinic: Glutamine Metabolism to Cancer Therapy,” Nat. Rev. Cancer 16:619-634 (2016)). Expression of GLS is regulated by oncogenic transcription factors, and its role in cancer is well characterized and supportive of tumorigenesis (Gao et al., “c-Myc Suppression of miR-23a/b Enhances Mitochondrial Glutaminase Expression and Glutamine Metabolism,” Nature 458:762-765 (2009); Lukey et al., “The Oncogenic Transcription Factor c-Jun Regulates Glutaminase Expression and Sensitizes Cells to Glutaminase-Targeted Therapy,” Nat. Commun. 7:11321 (2016)).

The function of GLS2 in cancer is less well defined and appears to be context dependent. The GLS2 gene is a transcriptional target of p53 (Hu et al., “Glutaminase 2, a Novel p53 Target Gene Regulating Energy Metabolism and Antioxidant Function,” Proc. Natd. Acad. Sci. U.S.A. 107:7455-7460 (2010); Suzuki et al., “Phosphate-Activated Glutaminase (GLS2), a p53-Inducible Regulator of Glutamine Metabolism and Reactive Oxygen Species,” Proc. Natl. Acad. Sci. U.S.A. 107:7461-7466 (2010)), and in glioblastoma and liver cancer GLS2 has been described as a tumor suppressor (Mates et al., “Glutaminase Isoenzymes in the Metabolic Therapy of Cancer,” Biochim. Biophys. Acta—Rev. Cancer 1870:158-164 (2018)). However, GLS2 expression is also regulated by oncoproteins including N-myc (Xiao et al., “Myc Promotes Glutaminolysis in Human Neuroblastoma Through Direct Activation of Glutaminase 2,” Oncotarget 6:40655-40666 (2015)), and was identified as one of only 16 essential metabolic genes in a functional genomics screen (Possemato et al., “Functional Genomics Reveal that the Serine Synthesis Pathway is Essential in Breast Cancer,” Nature 476:346-350 (2011)).

Consequently, GLS has been investigated as a possible drug target for cancer therapy, and an allosteric inhibitor, CB-839, is currently being evaluated in clinical trials (Gross et al., “Antitumor Activity of the Glutaminase Inhibitor CB-839 in Triple-negative Breast Cancer,” Mol. Cancer Ther. 13:890-901 (2014)). However, CB-839 has only shown high potency in cancers which depend upon GLS. Cancers which utilize GLS2, such as receptor positive breast cancers, are largely resistant to the compound, despite requiring the action of glutaminase enzymes to survive.

The present application is directed to overcoming these and other deficiencies in the art.

### SUMMARY

A first aspect of the present application relates to a method of reducing the production of glutamate from glutamine by GLS and by GLS2 in a cancerous cell or cancerous tissue. This method includes inhibiting glutaminase activity of GLS and GLS2 in the cancerous cell or cancerous tissue by a method involving selecting a cancerous cell or cancerous tissue; and contacting GLS and GLS2 in the cell or tissue with a dual GLS/GLS2 inhibitor; where the contacting reduces the production of glutamate from glutamine by GLS and by GLS2 in the cell or tissue.

A second aspect of the present application relates to a method of treating a subject with a condition mediated by production of glutamate from glutamine by GLS and by GLS2. This method includes selecting a subject with a condition mediated by production of glutamate from glutamine by GLS and by GLS2 and administering to the selected subject a dual GLS/GLS2 inhibitor under conditions effective to treat the condition mediated by production of glutamate from glutamine.

A third aspect of the present application relates to a method of reducing the production of glutamate from glutamine by GLS2 in a cancerous cell or cancerous tissue. This method includes inhibiting glutaminase activity of GLS2 in the cancerous cell or cancerous tissue by a method involving selecting a cancerous cell or cancerous tissue characterized by GLS2 overexpression and/or GLS2 hyperactivity; and contacting GLS2 in the cell or tissue with an inhibitor of GLS2 glutaminase activity; where the contacting reduces the production of glutamate from glutamine by GLS2 in the cell or tissue.

A fourth aspect of the present application relates to a method of treating a subject with a condition mediated by production of glutamate from glutamine by GLS2. This method includes selecting a subject with a condition mediated by production of glutamate from glutamine by GLS2 and administering to said selected subject an inhibitor of GLS2 glutaminase activity under conditions effective to treat the condition mediated by production of glutamate from glutamine.

Efforts to target glutamine metabolism for cancer therapy have focused on the glutaminase isozyme GLS. The importance of the other isozyme, GLS2, in cancer has remained unclear, and it has been described as a tumor suppressor in some contexts. As demonstrated herein, it has been determined that GLS2 is upregulated and essential in luminal-subtype breast tumors, which account for >70% of breast cancer incidence. GLS2 expression is elevated by GATA3 in luminal-subtype cells but suppressed by promoter methylation in basal-subtype cells. Although luminal breast cancers resist GLS-selective inhibitors, it was found that they can be targeted with a dual-GLS/GLS2 inhibitor. These results establish a critical role for GLS2 in mammary tumorigenesis and advance the understanding of how to target glutamine metabolism in cancer generally.

It is reported herein that basal- and luminal-subtype breast cancers employ different strategies for glutamine catabolism, impacting their sensitivity profiles to glutaminase inhibitors. Elevated GLS2 expression in luminal-subtype cancers is driven in part by GATA3. Targeting GLS2 with the pan-glutaminase inhibitor 968 inhibits luminal-subtype breast cancer cell proliferation and tumorigenesis (FIG. 1).

A critical onco-supportive role for GLS2 in breast cancer is described herein. It is demonstrated that the expression of the GLS2 gene is regulated by GATA3, and that the gene product is essential for cell proliferation and tumorigenesis in luminal-subtype breast cancers, which account for ˜75% of total breast cancer incidence (Table 1) (Dai et al., “Breast Cancer Intrinsic Subtype Classification, Clinical Use and Future Trends,” Am. J. Cancer Res. 5:2929-2943 (2015), which is hereby incorporated by reference in its entirety). Moreover, it is shown that GLS2 can be targeted with the small-molecule inhibitor 968 to suppress tumorigenesis and overcome resistance to GLS-selective inhibitors. These findings establish a previously unappreciated, essential, role for GLS2 in breast cancer biology and provide important insights regarding how to target glutamine metabolism for cancer therapy.

TABLE 1
The molecular subtypes of breast cancer.

| | Typical receptor | | |
|---|---|---|---|
| Molecular subtype | staining | Other markers | Relative incidence |
| Luminal A | ER+ and/or PR+, | Low Ki67 (<14%) | 50-60% |
| | HER2− | | |
| Luminal B | ER+ and/or PR+, | High Ki67 (>14%) | 10-20% |
| | HER2+/− | | |
| HER2+ | ER−, PR−, HER2+ | | ~15% |
| Basal | ER−, PR−, HER2− | | ~15% |

The present application demonstrates, inter alia, the role of GLS and GLS2 in breast cancers, that the inhibitor 968 is a dual GLS/GLS2 inhibitor, and that compound 968 can be used to target GLS and GLS2 in breast cancers. Based on the findings described herein, it is expected that other dual GLS/GLS2 inhibitors (including other compounds derived from 968 known to inhibit GLS) can be used to treat other cancers in which GLS2 is active, including other cancers resistant to GLS-selective inhibitors such as BPTES and/or CB-839.

### BRIEF DESCRIPTION OF THE DRAWINGS

FIG. 1 depicts the different strategies for glutamine catabolism between basal- and luminal-subtype breast cancers.

FIG. 2 is a table of the reagents, sources and their identifiers that were used in this application.

FIGS. 3A-3C depict the knockdown of GLS2 or GLS disrupting glutamine-mediated anaplerosis in luminal- and basal-subtype cells, respectively. FIG. 3A is the graphical depiction of the fold changes in total abundance of TCA cycle metabolites in MDA-MB-453 and MDA-MB-231 cells resulting from knockdown of GLS2 and GLS, respectively. Mean±SEM of triplicates relative to the control. FIG. 3B is the graphical depiction of the fold changes in total abundance of TCA cycle metabolites in MDA-MB-453 and MDA-MB-231 cells caused by knockdown of GLS-targeted and GLS2-targeted shRNAs, respectively. Mean±SEM of triplicates relative to the control. FIG. 3C is the graph of the rescue of the inhibitory effects of glutaminase-knockdowns on cell proliferation by supplementation of culture medium with 2 mM dimethyl-α-KG. Each panel shows the percent of cell growth in cell lines transfected with either a control siRNA (“C”), or two independent cell lines transfected with siRNAs simultaneously targeting GLS2 and GLS.

FIGS. 4A-4D show that the luminal-subtype breast cancer cells use glutamine to supply the TCA cycle, but resist GLS inhibitors. FIG. 4A are graphs depicting the effect of the GLS inhibitors BPTES and CB-839 on proliferation of basal-subtype (MDA-MB-231 and TSE) and luminal-subtype (MDA-MB-453 and T-47D) breast cancer cells over 6 days. Mean±SD of triplicate assays. FIG. 4B is a graphical depiction of glutamine consumption rates, per mg of total cellular protein, of breast cancer cell lines. Mean±SD of biological triplicates. FIG. 4C is a western blot showing relative levels of SLC1A5 in breast cancer cell lines. (Note that SLC1A5 is an integral membrane protein subject to covalent post-translational modifications including glycosylations, which cause it to run at a range of molecular weights on SDS-PAGE.) FIG. 4D is the graph of the abundance ratios of fully labeled intracellular glutamine, glutamate, and TCA cycle metabolites in breast cancer cells supplied with [U-¹³C]-glutamine for 10 hours. Mean±SEM of biological triplicate samples.

FIGS. 5A-5C relate to TCA cycle anaplerosis in breast cancer cells. FIG. 5A is a schematic depicting the labeling of TCA cycle intermediates by a [U-¹³C]-glutamine tracer. The first turn of the TCA cycle in the oxidative direction generates m+4 citrate/isocitrate. In contrast, reductive carboxylation of glutamine-derived α-ketoglutarate (α-KG) generates m+5 citrate/isocitrate. FIG. 5B is the graph of the relative intracellular abundance of the TCA cycle metabolites α-KG, fumarate, and citrate/isocitrate in breast cancer cell lines. Mean±SD of biological triplicate samples. FIG. 5C is a graph of the abundance ratios of fully-labeled intracellular glutamate and α-KG in breast cancer cells supplied with [U-¹³C]-glutamine for 1 hour (left bar for each metabolite) or 24 hours (right bar for each metabolite). Mean±SEM of biological triplicate samples.

FIGS. 6A-6G show that GLS2 is upregulated in luminal-subtype breast cancers. FIG. 6A is box and whisker plots showing transcript levels of GLS2 (left panel) and GLS (right panel) in the molecular subtypes of breast cancer. RNA-Seq V2 RSEM data are from The Cancer Genome Atlas invasive breast cancer dataset. The mean expression in each group is indicated by a cross, and the box and whiskers indicate the minimum, first quartile, median, third quartile, and maximum values. **p≤0.01. FIG. 6B is a plot showing the relative GLS2 protein levels in tissue microarray slices of normal mammary tissue, receptor-positive, and receptor-negative breast tumors. **p≤0.01. FIG. 6C is microscopy images of breast tissue microarray slices stained brown for GLS2. Representative images are shown for normal breast tissue along with receptor-positive and receptor-negative breast tumors. Scale bars, 200 μm. Compared to the control tissue, which shows moderate brown staining, there is abundant brown staining in the receptor-positive breast tumor tissue but very little brown staining in the receptor-negative breast tumor tissue. FIG. 6D is a plot of the Quantitative RT-PCR data showing relative levels of GLS2 transcript in breast cancer cell lines. Reactions were carried out in triplicate, and error bars indicate the RQₘₐₓ and RQₘᵢₙ values. FIG. 6E is a plot of the quantitative RT-PCR data showing relative levels of GLS transcript in breast cancer cell lines. Reactions were carried out in triplicate, and error bars indicate the RQₘₐₓ and RQₘᵢₙ values. FIG. 6F is western blots showing relative levels of GLS and GLS2 in breast cancer cell lines. A non-specific band from the GLS2 antibody, clearly visible for MDA-MB-231 and Hs 578T lysates, is labeled N/S. FIG. 6G is western blots of whole-cell lysates (WCL), and cytosolic, mitochondrial, and nuclear fractions from MDA-MB-453 and MDA-MB-231 cells. VDAC, ASNS, and Lamin A serve as mitochondrial, cytosolic, and nuclear marker proteins, respectively.

FIGS. 7A-7D show that basal-subtype breast cancer cells express xCT and secrete glutamate, whereas luminal-subtype breast cancers have upregulated GLS2 which is localized to mitochondria. FIG. 7A is a mammary tissue microarray, probed for GLS2. The label ‘N’ indicates normal mammary tissue, ‘+’ indicates receptor-positive breast tumor tissue, and ‘−’ indicates receptor-negative breast tumor tissue. Unlabeled slices are of hyperplasia, non-malignant tumors, or sarcoma. FIG. 7B is western blots showing relative levels of SLC3A2 and SLC7A11, the heavy and light chains, respectively, of the xCT transporter in breast cancer cell lines. Both are integral membrane proteins subject to post-translational modifications including glycosylations, which cause them to run at a range of molecular weights on SDS-PAGE. FIG. 7C is a graph of the rate of glutamate secretion by breast cancer cell lines. Mean±SD of biological triplicate samples. FIG. 7D is immunofluorescence images of SK-BR-3 breast cancer cells ectopically expressing HA-tagged GLS2 and/or myc-tagged GLS. Hsp60 serves as a mitochondrial marker. Scale bars, 10 μm.

FIGS. 8A-8D relate to the analysis of the GLS2 gene promoter. FIG. 8A is the graph of the copy-number analysis showing copy-number gains and gene amplifications for GLS2 and GLS in the different subtypes of breast tumor. Data are from TCGA invasive breast cancer dataset. FIG. 8B shows approximately 3600 base-pairs (bp) of the GLS2 gene promoter region (SEQ ID NO:1). The CpG island is indicated by bold underlined text (i.e., from position −1040 relative to the transcription start site to position +1296 relative to the transcription start site). The predicted GATA3 binding site, the transcription start site (TSS), and the start codon are each highlighted. As close match to the GATA3 consensus motif is also shown (compare the predicted GATA3 binding site (residues 706-717 of SEQ ID NO:1) with consensus motif 1 (SEQ ID NO:2) and consensus motif 2 (SEQ ID NO:3)). FIG. 8C shows the position of the CpG island in the GLS2 gene promoter, centered around the TSS. FIG. 8D is a graph of the quantitative RT-PCR data showing relative levels of GLS2 transcript in breast cancer cell lines transfected with either a control siRNA (“C”) or two independent GATA3-targeted siRNAs (“GATA3 siRNA”). Reactions were carried out in triplicate, and error bars indicate the RQₘₐₓ and RQₘᵢₙ values.

FIGS. 9A-9F show that GLS2 gene expression is regulated by GATA3 and promoter methylation. FIG. 9A is western blots showing relative levels of GLS2, and previously identified transcription factors for the GLS2 gene, in breast cancer cell lines. A non-specific band is labeled N/S. FIG. 9B is western blots showing relative levels of GLS2, the luminal-transcription factor GATA3, and the receptors ERα, PR, and HER2 in breast cancer cell lines. A non-specific band is labeled N/S. FIG. 9C is a plot of GLS2 and GATA3 transcript levels in human breast tumors, with the Pearson correlation coefficient r indicated. RNA-Seq V2 RSEM data from TCGA invasive breast cancer dataset. FIG. 9D is a graphical analysis showing the quantitative RT-PCR data showing relative levels of a 176-bp fragment of the GLS2 gene promoter, centered on the putative GATA3 binding site, in chromatin immunoprecipitations (ChIPs) using negative control IgG or a GATA3-targeted antibody. Mean±SD of biological triplicate samples. FIG. 9E is western blots showing GLS2 and GATA3 levels in MDA-MB-453 and T-47D cells transfected with either a control siRNA (labeled C) or with two independent GATA3-targeted siRNAs. Bands in the GLS2 blot were quantified by densitometry using ImageJ, and relative band intensities are indicated above the blot. Since GATA3 regulates expression of Tubulin and several other cytoskeletal proteins, a non-specific (N/S) band is shown to demonstrate equal loading. FIG. 9F is heatmap visualizations showing relative methylation levels at CpG sites within the CpG island of the GLS2 gene promoter in breast cancer cell lines. Labels for the CpG sites refer to the amplicon in which the sites are located (amplicons 0 to 9) and then the position of the site within that amplicon (i.e. first CpG site, second CpG site, etc.). Numbers missing in the sequence are for sites at which methylation ratios could not be determined. Although the amplicons were designed to overlap, only a single reading is shown for sites covered by more than one amplicon. FIG. 9G is a western blot showing relative levels of GLS2 in TSE cells treated with different concentrations of the DNA hypomethylating agent azacitidine for 48 hours. Bands in the GLS2 blot were quantified by densitometry using ImageJ, and relative band intensities are indicated above the blot. A non-specific band is labeled N/S.

FIGS. 10A-10F show that GLS2 is essential in luminal-subtype breast cancers. FIG. 10A is western blots showing GLS and GLS2 levels in MDA-MB-453 cells and MDA-MB-231 cells expressing either a control shRNA or two independent GLS2-targeted (MDA-MB-453 cells) or GLS-targeted (MDA-MB-231 cells) shRNAs. FIG. 10B is a graph of the fold changes in fully-labeled glutamate and TCA cycle metabolites (i.e., derived directly from [U-¹³C]-glutamine) in MDA-MB-453 cells following knockdown of GLS2, or in MDA-MB-231 cells following knockdown of GLS. Mean±SEM of biological triplicates. FIG. 10C are graphs and western blots showing the effect of knocking down either GLS, GLS2, or both GLS and GLS2 simultaneously, on the proliferation of breast cancer cell lines over six days. For each condition, two independent siRNAs were used, and the effects were compared with those of a control siRNA (labeled C). Mean±SD of triplicate assays. Lower panels show western blots for GLS and GLS2 in each sample. *p≤0.05, **p≤0.01, ns=not significant. FIG. 10D is a western blot showing partial knockdown of GLS2 in MDA-MB-453 stably expressing two independent GLS2-targeted shRNAs, relative to cells stably expressing a control shRNA. FIG. 10E is a graphical analysis of the inhibition of proliferation over 6 days for MDA-MB-453 cells stably expressing GLS2-targeted shRNAs, relative to cells stably expressing a control shRNA (labeled C). Experiments were run either without (left panel) or with (right panel) supplementation of the culture medium with 2 mM dimethyl α-ketoglutarate (dm-α-KG). Mean±SD of triplicate assays. *p≤0.05, **p≤0.01, ns=not significant. FIG. 10F is a graph of the growth of xenograft tumors in mice by MDA-MB-453 cells stably expressing either a control shRNA or two independent GLS2-targeted shRNAs. Mean±SD (n=6 tumors per condition). *p≤0.05, **p≤0.01.

FIGS. 11A-11G show that GLS2 mediates resistance to BPTES. FIG. 11A is plots of real-time assays showing inhibition of recombinant GLS by different concentrations of BPTES (left panel) and CB-839 (right panel). FIG. 11B is plots of real-time assays showing resistance of full-length recombinant GLS2 to BPTES (left panel) and CB-839 (right panel). FIG. 11C is western blot standard curves to estimate absolute levels of GLS and GLS2 in breast cancer cells. Estimated levels per pg of total cellular protein are as follows: MDA-MB-453 GLS: 1.5 ng per 20 μg=75 pg/μg; MDA-MB-453 GLS2: 5.5 ng per 20 μg=275 pg/μg; MDA-MB-231 GLS: 10 ng per 20 μg=500 pg/μg; MDA-MB-231 GLS2: <75 pg/μg; T-47D GLS: 8 ng per 40 μg=200 pg/μg; T-47D GLS2: 8 ng per 40 μg=200 pg/μg; DU4475 GLS: 15 ng per 20 μg=750 pg/μg; DU4475 GLS2: 12.5 ng per 20 μg=625 pg/μg. FIG. 11D is the graphical analysis of the fold changes in total abundance of glutamine, glutamate, and TCA cycle metabolites in basal-subtype (upper panels) and luminal-subtype (lower panels) breast cancer cell lines following treatment with 10 μM BPTES. Mean±SEM of triplicates relative to the vehicle. FIG. 11E is the graphical analysis of the relative proliferation rates of basal-subtype breast cancer cells stably overexpressing GLS2 or carrying the plasmid vector only. Proliferation over 6 days was measured. Mean±SD of triplicate assays. All p values are slightly greater than 0.05, and changes are therefore classed as not significant (ns). FIG. 11F is western blots showing levels of GLS and GLS2 in DU4475 cells transfected with either a control siRNA (labeled C), two independent GLS-targeted siRNAs, two independent GLS2-targeted siRNAs, or GLS- and GLS2-targeted siRNAs simultaneously. FIG. 11G is a graphical depiction of the rescue of the inhibitory effects of glutaminase-knockdowns on cell proliferation by supplementation of culture medium with 2 mM dimethyl-α-KG.

FIGS. 12A-12H show that GLS2 expression is sufficient for resistance to GLS inhibitors. FIG. 12A is a graphical depiction of the inhibition of glutaminase activity by 10 μM BPTES in mitochondria isolated from breast cancer cell lines. The plot shows activity in the presence of 10 μM BPTES relative to matched samples with no BPTES present. Mean±SD of triplicate assays. FIG. 12B is a plot of the effect of increasing inorganic phosphate concentrations on the catalytic activity of recombinant full-length human GLS (GAC splice variant) and GLS2. Mean±SD of triplicate assays. FIG. 12C is the graph of the fold changes in fully labelled intracellular metabolites (i.e. derived directly from [U-¹³C]-glutamine) in basal-subtype (top) and luminal-subtype (bottom) breast cancer cell lines, following treatment with 10 μM BPTES. Mean±SEM of triplicates relative to the vehicle. FIG. 12D is western blots showing levels of endogenous GLS and ectopically-expressed V5-tagged GLS2 in MDA-MB-231 and TSE cells. FIG. 12E is the plot of the inhibition of proliferation over 6 days of MDA-MB-231 and TSE cells, stably expressing GLS2 (two clones for each cell line) or carrying the plasmid vector only, by treatment with different concentrations of BPTES. Mean±SD of triplicate assays. FIG. 12F is a plot of the effect of different concentrations of BPTES on proliferation of DU4475 cells over 6 days. Mean±SD of triplicate assays. FIG. 12G is western blots showing relative levels of GLS and GLS2 in MDA-MB-453, MDA-MB-231 and DU4475 cells. A non-specific band from the GLS antibody is marked N/S. FIG. 12H is the graphical analysis of the effect of knocking down either GLS, GLS2, or both GLS and GLS2 simultaneously, on the proliferation of DU4475 cells over six days. For each condition, two independent siRNAs were used, and the effects were compared with those of a control siRNA (labeled C). Mean±SD of triplicate assays. **p≤0.01, ns=not significant.

FIGS. 13A-13E show that 968 inhibits GLS2 and suppresses BPTES-resistant breast cancer growth. FIG. 13A is a plot of the inhibition of purified recombinant full-length human GLS (GAC splice variant) or GLS2 by different concentrations of 968. Mean±SD of triplicate assays. FIG. 13B is a plot of the effect of different concentrations of 968 or BPTES on proliferation of DU4475 cells over 6 days. Mean±SD of triplicate assays. FIG. 13C is a graph of the fold changes in fully labeled intracellular metabolites, derived directly from [U-¹³C]-glutamine, when breast cancer cells are treated with 10 μM 968 for the indicated periods of time. Mean±SEM of triplicates relative to the vehicle. (For each metabolite, the bars are, from left to right, vehicle, 10 hours, 24 hours, and 72 hours.) FIG. 13D is a plot of the growth of MDA-MB-453 xenograft tumors in mice. Once palpable tumors were detected (at Day 14), mice were divided into two groups, one of which received subcutaneous injections of 10 mg/kg 968 three times per week, while the other received carrier solution only. Mean±SD (n=6 tumors per condition). **p≤0.01. FIG. 13E is a plot showing the final size of MDA-MB-453 xenograft tumors following treatment of mice with 10 mg/kg 968, 10 mg/kg BPTES, or carrier solution only, three times per week from Day 14 until Day 35. Mice were sacrificed at Day 35 and tumors were excised prior to measurement. Mean±SD (n=6 tumors per condition). **p≤0.01, ns=not significant.

FIGS. 14A-14C show that the glutaminase inhibitor 968 suppresses proliferation and TCA cycle anaplerosis. FIG. 14A is a plot of the real-time effects of 968 and the inactive analog compound 26 on GLS2 activity (measured by NADH fluorescence). FIG. 14B is plots of the dose curves showing inhibition of breast cancer cell proliferation over 6 days by different concentrations of 968. Mean±SD of triplicate assays. FIG. 14C is a graphical analysis of the fold changes in total metabolite abundances in MDA-MB-231 (upper panel) and MDA-MB-453 (lower panel) cells following treatment with 10 μM 968 for different periods of time. Mean±SEM of triplicates relative to the vehicle.

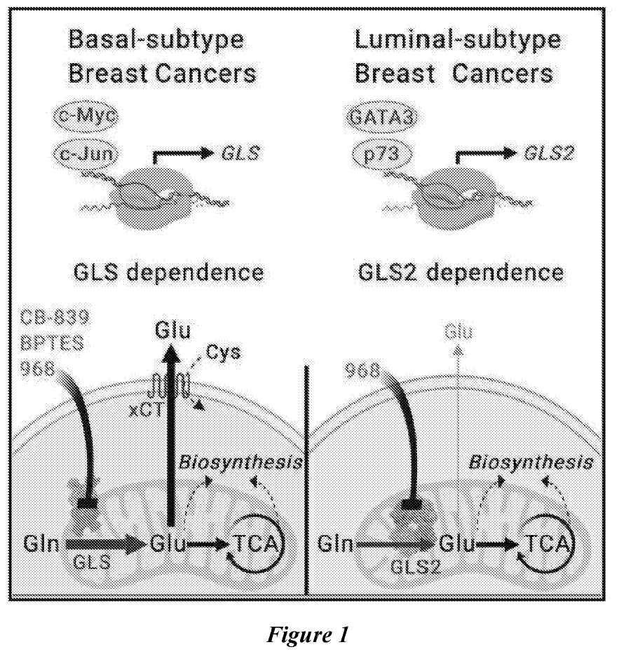

### DETAILED DESCRIPTION

One aspect of the present application relates to a method of reducing the production of glutamate from glutamine by GLS2 in a cancerous cell or cancerous tissue. This method includes inhibiting glutaminase activity of GLS2 in the cancerous cell or cancerous tissue by a method involving selecting a cancerous cell or cancerous tissue characterized by GLS2 overexpression and/or GLS2 hyperactivity; and contacting GLS2 in the cell or tissue with an inhibitor of GLS2 glutaminase activity; where the contacting reduces the production of glutamate from glutamine by GLS2 in the cell or tissue.

Another aspect of the present application relates to a method of reducing the production of glutamate from glutamine by GLS and by GLS2 in a cancerous cell or cancerous tissue. This method includes inhibiting glutaminase activity of GLS and GLS2 in the cancerous cell or cancerous tissue by a method involving selecting a cancerous cell or cancerous tissue; and contacting GLS and GLS2 in the cell or tissue with a dual GLS/GLS2 inhibitor; where the contacting reduces the production of glutamate from glutamine by GLS and by GLS2 in the cell or tissue.

Another aspect of the present application relates to a method of treating a subject with a condition mediated by production of glutamate from glutamine by GLS2. This method includes selecting a subject with a condition mediated by production of glutamate from glutamine by GLS2, and administering to said selected subject an inhibitor of GLS2 glutaminase activity under conditions effective to treat the condition mediated by production of glutamate from glutamine.

A second aspect of the present application relates to a method of treating a subject with a condition mediated by production of glutamate from glutamine by GLS and by GLS2. This method includes selecting a subject with a condition mediated by production of glutamate from glutamine by GLS and by GLS2 and administering to the selected subject a dual GLS/GLS2 inhibitor under conditions effective to treat the condition mediated by production of glutamate from glutamine.

The term “reducing” means to suppress, decrease, diminish, or lower the production of glutamate from glutamine. The term “treatment” or “treating” means any manner in which one or more of the symptoms of a disease or disorder are ameliorated or otherwise beneficially altered. Treatment also encompasses any pharmaceutical use of the compositions herein, such as use for treating conditions mediated by the production of glutamate from glutamine by GLS2 alone or by both GLS and GLS2.

The term “GLS” refers to kidney-type glutaminases encoded by a GIS gene, including the isoforms GAC and KGA. The term “GLS2” refers to liver-type glutaminases encoded by a GLS2 gene, including the isoforms LGA and GAB.

The methods of the present application involve the use of an inhibitor of GLS2 glutaminase activity. Such inhibitors include any inhibitor that is capable of inhibiting glutaminase activity of at least one GLS2 isoform (e.g., LGA and/or GAB). In at least some embodiments of all aspects of the present application, the inhibitor of GLS2 glutaminase activity is a dual GLS/GLS2 inhibitor. A “dual GLS/GLS2 inhibitor” is a compound that is capable of inhibiting glutaminase activity of at least one GLS isoform (e.g., GAC and/or KGA) and at least one GLS2 isoform (e.g., LGA and/or GAB), in any combination.

In at least one embodiment of all aspects of the present application, the inhibitor is a compound (or a pharmaceutically acceptable salt, ester, enol ether, enol ester, solvate, hydrate, or prodrug thereof) selected from the group consisting of:
I) compounds of Formula IA:

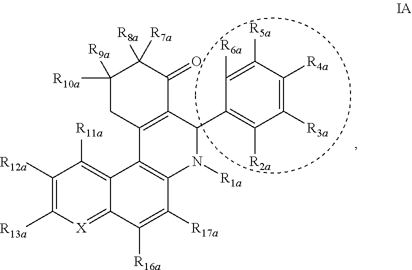

wherein:
the dotted circle identifies an active moiety;
X is independently —CR₁₄ₐ— or N;
R₁ₐ is independently H, —OH, —OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₂ₐ, R₃ₐ, R₄ₐ, R₅ₐ, and R₆ₐ are each independently a photoreactive moiety, H, halogen, —NO₂, —OH, —OR₁₄ₐ, —SR₁₄ₐ, —NH₂, —NHR₁₄ₐ, —NR₁₄ₐR₁₅ₐ, R₁₄ₐC(O)—, R₁₄ₐOC(O)—, R₁₄ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; or R₂ₐ and R₃ₐ, R₃ₐ and R₄ₐ, R₄ₐ and R₅ₐ, or R₅ₐ and R₆ₐ are combined to form a heterocyclic ring optionally substituted with a photoreactive moiety;
R₇ₐ, R₈ₐ, R₉ₐ, and R₁₀ₐ are each independently a photoreactive moiety, H, —OH, —NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl are optionally substituted from 1 to 3 times with substituents selected from the group consisting of, halogen, —OH, —NH₂, C₁-C₆ alkyl, C₂-C₆ alkenyl, C₁-C₆ alkoxy, —SH, and C₁-C₆ thioalkyl, and wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; and
R₁₁ₐ, R₁₂ₐ, R₁₃ₐ, R₁₄ₐ, R₁₅ₐ, R₁₆ₐ, and R₁₇ₐ are each independently a photoreactive moiety, H, halogen, —OH, —O—C₁-C₆ alkyl, —O—C₂-C₆ alkenyl, —O—C₂-C₆ alkynyl, —NO₂, C₁-C₆ alkyl, C₂-C₆ alkenyl, C₂-C₆ alkynyl, C₃₋₆ cycloalkyl, C₄-C₇ cycloalkylalkyl, aryl C₁-C₆ alkyl, mono or polycyclic aryl, wherein the alkyl, alkenyl, alkynyl, cycloalkyl, cycloalkylalkyl, arylalkyl, and mono or polycyclic aryl are optionally substituted with a photoreactive moiety and each one of R₁₁ₐ-R₁₇ₐ is optionally substituted with —NH₂, —OH, halogen, —COOH, —NO₂, or —CN;
II) compounds of Formula IB:

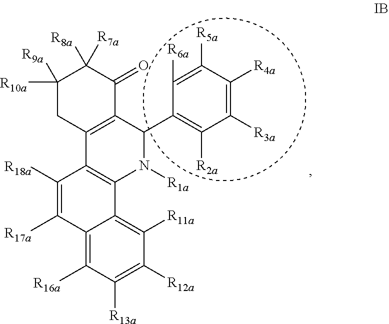

wherein:
the dotted circle identifies an active moiety;
R₁ₐ is H, —OH, —OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₂ₐ, R₃ₐ, R₄ₐ, R₅ₐ, and R₆ₐ are each independently a photoreactive moiety, H, halogen, —NO₂, —OH, —OR₁₄ₐ, —SR₁₄ₐ, —NH₂, —NHR₁₄ₐ, —NR₁₄ₐR₁₅ₐ, R₁₄ₐC(O)—, R₁₄ₐOC(O)—, R₁₄ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; or R₂ₐ and R₃ₐ, R₃ₐ and R₄ₐ, R₄ₐ and R₅ₐ, or R₅ₐ and R₆ₐ are combined to form a heterocyclic ring optionally substituted with a photoreactive moiety; wherein at least two of R₂, R₃ₐ, R₄ₐ, R₅ₐ, and R₆ₐ are not hydrogen;
R₇ₐ, R₈ₐ, R₉ₐ, and R₁₀ₐ are each independently a photoreactive moiety, H, —OH, —NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl are optionally substituted from 1 to 3 times with substituents selected from the group consisting of, halogen, —OH, —NH₂, C₁-C₆ alkyl, C₂-C₆ alkenyl, C₁-C₆ alkoxy, —SH, and C₁-C₆ thioalkyl, and wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; and
R₁₁ₐ, R₁₂ₐ, R₁₃ₐ, R₁₄ₐ, R₁₅ₐ, R₁₆ₐ, R₁₇ₐ, and R₁₈ₐ are each independently a photoreactive moiety, H, halogen, —OH, —O—C₁-C₆ alkyl, —O—C₂-C₆ alkenyl, —O—C₂-C₆ alkynyl, —NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, wherein the alkyl, alkenyl, alkynyl, cycloalkyl, cycloalkylalkyl, arylalkyl, and mono or polycyclic aryl are optionally substituted with a photoreactive moiety and each one of R₁₁ₐ-R₁₈ₐ is optionally substituted with —NH₂, —OH, halogen, —COOH, —NO₂, or —CN;
III) compounds of Formula IC:

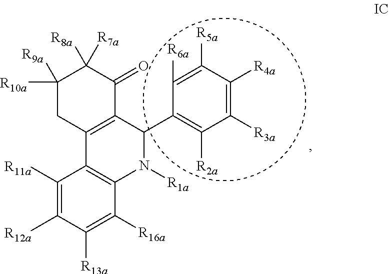

wherein:
the dotted circle identifies an active moiety;
R₁ₐ is H, —OH, —OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₂ₐ, R₃ₐ, R₄ₐ, R₅ₐ, and R₆ₐ are each independently a photoreactive moiety, H, halogen, —NO₂, —OH, —OR₁₄ₐ, —SR₁₄ₐ, —NH₂, —NHR₁₄ₐ, —NR₁₄ₐR₁₅ₐ, R₁₄ₐC(O)—, R₁₄ₐOC(O)—, R₁₄ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; or R₂ₐ and R₃ₐ, R₃ₐ and R₄ₐ, R₄ₐ and R₅ₐ, or R₅ₐ and R₆ₐ are combined to form a heterocyclic ring optionally substituted with a photoreactive moiety; wherein at least two of R₂ₐ, R₃ₐ, R₄ₐ, R₅ₐ, and R₆ₐ are not hydrogen;
R₇ₐ, R₈ₐ, R₉ₐ, and R₁₀ₐ are each independently a photoreactive moiety, H, —OH, —NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl are optionally substituted from 1 to 3 times with substituents selected from the group consisting of, halogen, —OH, —NH₂, C₁-C₆ alkyl, C₂-C₆ alkenyl, C₁-C₆ alkoxy, —SH, and C₁-C₆ thioalkyl, and wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; and
R₁₁ₐ, R₁₂ₐ, R₁₃ₐ, R₁₄ₐ, R₁₅ₐ, and R₁₆ₐ are each independently a photoreactive moiety, H, halogen, —OH, —O—C₁-C₆ alkyl, —O—C₂-C₆ alkenyl, —O—C₂-C₆ alkynyl, —NO₂, C₁-C₆ alkyl, C₂-C₆ alkenyl, C₂-C₆ alkynyl, C₃-C₇ cycloalkyl, C₄-C₇ cycloalkylalkyl, aryl C₁-C₆ alkyl, mono or polycyclic aryl, wherein the alkyl, alkenyl, alkynyl, cycloalkyl, cycloalkylalkyl, arylalkyl, and mono or polycyclic aryl are optionally substituted with a photoreactive moiety and each one of R₁₁ₐ—R₁₆ₐ is optionally substituted with —NH₂, —OH, halogen, —COOH, —NO₂, or —CN;
IV) compounds of Formula ID:

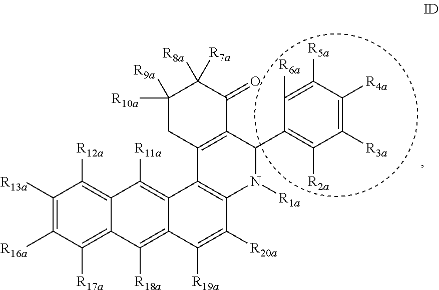

wherein:
the dotted circle identifies an active moiety;
R₁ₐ is H, —OH, —OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₂ₐ, R₃ₐ, R₄ₐ, R₅ₐ, and R₆ₐ are each independently a photoreactive moiety, H, halogen, —NO₂, —OH, —OR₁₄ₐ, —SR₁₄ₐ, —NH₂, —NHR₁₄ₐ, —NR₁₄ₐR₁₅ₐ, R₁₄ₐC(O)—, R₁₄ₐOC(O)—, R₁₄ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; or R₂ₐ and R₃ₐ, R₃ₐ and R₄ₐ, R₄ₐ and R₅ₐ, or R₅ₐ and R₆ₐ are combined to form a heterocyclic ring optionally substituted with a photoreactive moiety; wherein at least two of R₂ₐ, R₃ₐ, R₄ₐ, R₅ₐ, and R₆ₐ are not hydrogen;
R₇ₐ, R₈ₐ, R₉ₐ, and R₁₀ₐ are each independently a photoreactive moiety, H, —OH, —NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆alkyl are optionally substituted from 1 to 3 times with substituents selected from the group consisting of, halogen, —OH, —NH₂, C₁-C₆ alkyl, C₂-C₆ alkenyl, C₁-C₆ alkoxy, —SH, and C₁-C₆ thioalkyl, and wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; and
R₁₁ₐ, R₁₂ₐ, R₁₃ₐ, R₁₄ₐ, R₁₅ₐ, R₁₆ₐ, R₁₇ₐ, R₁₈ₐ, R₁₉ₐ and R₂₀ₐ are each independently a photoreactive moiety, H, halogen, —OH, —O—C₁-C₆ alkyl, —O—C₂-C₆ alkenyl, —O—C₂-C₆ alkynyl, —NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, wherein the alkyl, alkenyl, alkynyl, cycloalkyl, cycloalkylalkyl, arylalkyl, and mono or polycyclic aryl are optionally substituted with a photoreactive moiety and each one of R₁₁ₐ-R₂₀ₐ is optionally substituted with —NH₂, —OH, halogen, —COOH, —NO₂, or —CN;
V) compounds of Formula II:

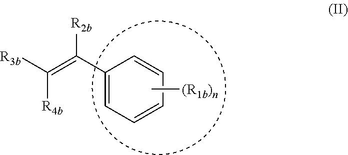

wherein:
the dotted circle identifies an active moiety;
n is an integer from 1 to 4;
R_{1b} is independently at each occurrence H, OH, OR_{5b}, halogen, CN, NO₂, NH₂, NHR_{5b}, NR_{5b}R_{6b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen;
R_{2b} is independently H, halogen, C₁-C₆ alkyl, C₂-C₆ alkenyl, C₂-C₆ alkynyl, C₃-C₆ cycloalkyl, C₄-C₇ cycloalkylalkyl, or mono or polycyclic aryl;
R_{3b} and R_{4b} are independently H, OR_{5b}, SR_{5b}, R_{5b}S(O)—, R_{5b}S(O)₂—, —COOR_{5b}, —C(O)NR_{5b}R_{6b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; or
R_{3b} and R_{4b} can combine together to form a mono or polycyclic heterocyclyl or heteroaryl containing from 1-5 heteroatoms selected from the group consisting of nitrogen, sulfur, and oxygen, each formed heteroaryl or heterocyclyl optionally substituted with substituents selected from the group consisting of oxo, thio, amino, C₁-C₆ alkyl, C₂-C₆ alkenyl, and C₂-C₆ alkynyl; and
R_{5b} and R_{6b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, each one of R_{5b} or R_{6b} optionally substituted from 1 to 3 times with substituents selected from the group consisting of H, C₁-C₆ alkyl, C₂-C₆ alkenyl, C₂-C₆ alkynyl, C₃-C₆ cycloalkyl, and C₄-C₇ cycloalkylalkyl;
VI) compounds of Formula III:

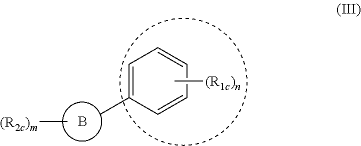

wherein:
the dotted circle identifies an active moiety;
m and n are integers from 1 to 4;
B is a substituted or unsubstituted mono or polycyclic aryl or mono or polycyclic heterocyclyl or heteroaryl with each cyclic unit containing from 1 to 5 heteroatoms selected from the group consisting of nitrogen, sulfur, and oxygen;
R_{1c} and R_{2c} are independently H, OH, OR_{3c}, halogen, CO, CN, NO₂, COOH, NH₂, NHR_{3c}, NR_{3c}R_{4c}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3c} and R_{4c} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen; and
VII) compounds of Formula IV

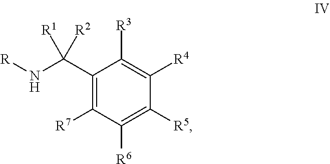

wherein:
R is selected from the group consisting of monocyclic or bicyclic aryl, monocyclic or bicyclic heteroaryl, and monocyclic or bicyclic heterocyclyl, wherein each monocyclic or bicyclic aryl, monocyclic or bicyclic heteroaryl, and monocyclic or bicyclic heterocyclyl can be optionally substituted from 1 to 4 times with substituents independently selected at each occurrence thereof from the group consisting of H, halogen, C₁₋₆ alkyl, aryl, —OR⁸, —CF₃, and —CHF₂;
R¹ and R² are each independently selected from the group consisting of a photoreactive moiety, H, halogen, and C₁₋₆ alkyl; or R¹ and R² are combined to form ═O;
R³, R⁴, R⁵, R⁶, and R⁷ are each independently selected from the group consisting of a photoreactive moiety, H, halogen, —NO₂, —NR⁸R⁹, —SO₂NR⁸R⁹, —N₃, —C(O)R⁸, aryl, heteroaryl, heterocyclyl, and

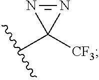 [SMILES: *C1(C(F)(F)F)N=N1]

and
R⁸ and R⁹ are each independently selected from the group consisting of H, C₁₋₆ alkyl, C₂₋₆ alkenyl, C₂₋₆ alkynyl, and aryl; or R⁸ and R⁹ are combined with the nitrogen to which they are attached to form a heterocyclyl, wherein the heterocyclyl can be optionally substituted with —COOH or —COOMe.

The term “halo” or “halogen” means fluoro, chloro, bromo, or iodo.

The term “optionally substituted” indicates that a group may have a substituent at each substitutable atom of the group (including more than one substituent on a single atom), and the identity of each substituent is independent of the others.

The term “substituted” or “substitution” of an atom means that one or more hydrogen on the designated atom is replaced with a selection from the indicated group, provided that the designated atom's normal valency is not exceeded. “Unsubstituted” atoms bear all of the hydrogen atoms dictated by their valency. When a substituent is oxo (i.e., ═O), then 2 hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent. Exemplary substituents include, without limitation, oxo, thio (i.e. ═S), nitro, cyano, halo, OH, NH2, C1-C6 alkyl, C1-C6 alkoxy, C2-C6 alkenyl, C2-C6 alkynyl, C3-C6 cycloalkyl, C4-C7 cycloalkylalkyl, monocyclic aryl, monocyclic heteroaryl, polycyclic aryl, and polycyclic heteroaryl.

The term “monocyclic” indicates a molecular structure having one ring.

The term “polycyclic” indicates a molecular structure having two (“bicyclic”) or more rings, including, but not limited to, fused, bridged, or spiro rings.

The term “alkyl” means an aliphatic hydrocarbon group which may be straight or branched having about 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “thioalkyl” means an alkyl group as described above bonded through a sulfur linkage.

The term “alkenyl” means an aliphatic hydrocarbon group containing a carbon-carbon double bond and which may be straight or branched having about 2 to about 6 carbon atoms in the chain. Preferred alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl.

The term “alkynyl” means an aliphatic hydrocarbon group containing a carbon-carbon triple bond and which may be straight or branched having about 2 to about 6 carbon atoms in the chain. Preferred alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl.

The term “alkoxy” means an alkyl-O—, alkenyl-O—, or alkynyl-O— group wherein the alkyl, alkenyl, or alkynyl group is described above. Exemplary alkoxy groups include methoxy, ethoxy, n-propoxy, i-propoxy, n-butoxy, pentoxy, and hexoxy.

The term “cycloalkyl” refers to a non-aromatic saturated or unsaturated mono- or polycyclic ring system which may contain 3 to 6 carbon atoms; and which may include at least one double bond. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, and syn-bicyclopropane.

The term “cycloalkylalkyl” refers to a radical of the formula —RaRb where Ra is an alkyl radical as defined above and Rb is a cycloalkyl radical as defined above. The alkyl radical and the cycloalkyl radical may be optionally substituted as defined above.

The term “aryl” refers to aromatic monocyclic or polycyclic ring system containing from 6 to 19 carbon atoms, where the ring system may be optionally substituted. Aryl groups of the present application include, but are not limited to, groups such as phenyl, naphthyl, azulenyl, phenanthrenyl, anthracenyl, fluorenyl, pyrenyl, triphenylenyl, chrysenyl, and naphthacenyl.

The term “arylalkyl” refers to a radical of the formula —RaRb where Ra is an alkyl radical as defined above and Rb is an aryl radical as defined above. The alkyl radical and the cycloalkyl radical may be optionally substituted as defined above.

The term “arylarylalkyl” refers to a radical of the formula —RaRbRc where Ra is an alkyl as defined above, Rb is an aryl radical as defined above, and Rc is an aryl radical as defined above. The alkyl radical and both aryl radicals may be optionally substituted as defined above.

The term “heterocyclyl” refers to a stable 3- to 18-membered ring radical which consists of carbon atoms and from one to five heteroatoms selected from the group consisting of nitrogen, oxygen and sulfur. For purposes of this application, the heterocyclyl radical may be a monocyclic, or a polycyclic ring system, which may include fused, bridged, or spiro ring systems; and the nitrogen, carbon, or sulfur atoms in the heterocyclyl radical may be optionally oxidized; the nitrogen atom may be optionally quaternized; and the ring radical may be partially or fully saturated. Examples of such heterocyclyl radicals include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone.

The term “heteroaryl” refers to an aromatic ring radical which consists of carbon atoms and from one to five heteroatoms selected from the group consisting of nitrogen, oxygen, and sulfur. For purposes of this application the heteroaryl may be a monocyclic or polycyclic ring system; and the nitrogen, carbon, and sulfur atoms in the heteroaryl ring may be optionally oxidized; the nitrogen may optionally be quaternized. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl.

Further heterocycles and heteroaryls are described in COMPREHENSIVE HETEROCYCLIC CHEMISTRY: THE STRUCTURE, REACTIONS, SYNTHESIS AND USE OF HETEROCYCLIC COMPOUNDS Vol. 1-8 (Alan R. Katritzky et al. eds., 1st ed. 1984), which is hereby incorporated by reference in its entirety.

A “photoreactive moiety” as used herein is a moiety that becomes reactive when exposed to ultraviolet or visible light. Suitable photoreactive moieties include, for example, aryl azides, diazirines, and benzophenone; when the photoreactive moiety is an aryl azide or benzophenone, it is attached to an aromatic ring. Suitable examples include, without limitation, —N═N⁺═N⁻:

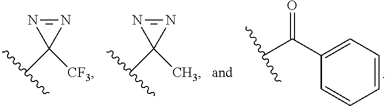 [SMILES: *C1(C(F)(F)F)N=N1 ; *C1(C)N=N1 ; "and" ; *C(=O)c1ccccc1]

Pharmaceutically acceptable salts include, but are not limited to, amine salts, such as but not limited to, N, N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine, and tris(hydroxymethyl) aminomethane; alkali metal salts, such as but not limited to, lithium, potassium, and sodium; alkali earth metal salts, such as but not limited to, barium, calcium, and magnesium; transition metal salts, such as but not limited to, zinc; and other metal salts, such as but not limited to, sodium hydrogen phosphate and disodium phosphate; and also including, but not limited to, salts of mineral acids, such as but not limited to, hydrochlorides and sulfates; and salts of organic acids, such as but not limited to, acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, cycloalkyl and heterocyclyl esters of acidic groups, including, but not limited to, carboxylic acids, phosphoric acids, phosphinic acids, sulfonic acids, sulfinic acids, and boronic acids. Pharmaceutical acceptable enol ethers include, but are not limited to, derivatives of formula C═C (OR) where R is hydrogen, alkyl, alkenyl, alkynyl, aryl, heteroaryl, cycloalkyl, or heterocyclyl. Pharmaceutically acceptable enol esters include, but are not limited to, derivatives of formula C═C (OC(O) R) where R is hydrogen, alkyl, alkenyl, alkynyl, aryl, heteroaryl, cycloalkyl, or heterocyclyl. Pharmaceutical acceptable solvates and hydrates are complexes of a compound with one or more solvent or water molecules, or 1 to about 100, or 1 to about 10, or one to about 2, 3 or 4, solvent or water molecules.

In accordance with some embodiments of the inhibitor compound of Formula III,

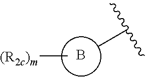

has the formula:

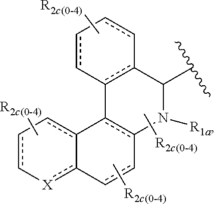

where
X is carbon or nitrogen;
R₁ₐ is H, OH, OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₁₄ₐ is H, halogen, OH, NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, or mono or polycyclic aryl, with R₁₄ₐ is optionally substituted with NH₂, OH, halogen, COOH, NO₂, or CN; and
the total number of R_{2c} substituents is from 1 to 4.

In accordance with some embodiments of the compounds of Formula IA, compounds of Formula IB, compounds of Formula IC, compounds of Formula ID, compounds of Formula II, and compounds of Formula III, R₄ₐ is not hydrogen. In a further embodiment, at least two of R₂ₐ, R₃ₐ, R₄ₐ, R₅ₐ, and R₆ₐ are not hydrogen. In another embodiment, at least two of R₃ₐ, R₄ₐ, and R₅ₐ are not hydrogen.

In at least one embodiment of the compounds of Formula IV, R⁵ is not hydrogen. In a further embodiment, at least two of R³, R⁴, R⁵, R⁶, and R⁷ are not hydrogen. In another embodiment, at least two of R⁴, R⁵, and R⁶ are not hydrogen.

With respect to compounds of Formula IA, Formula IB, Formula IC, Formula ID, Formula II, Formula III, and Formula IV, suitable active moieties include, but are not limited to:

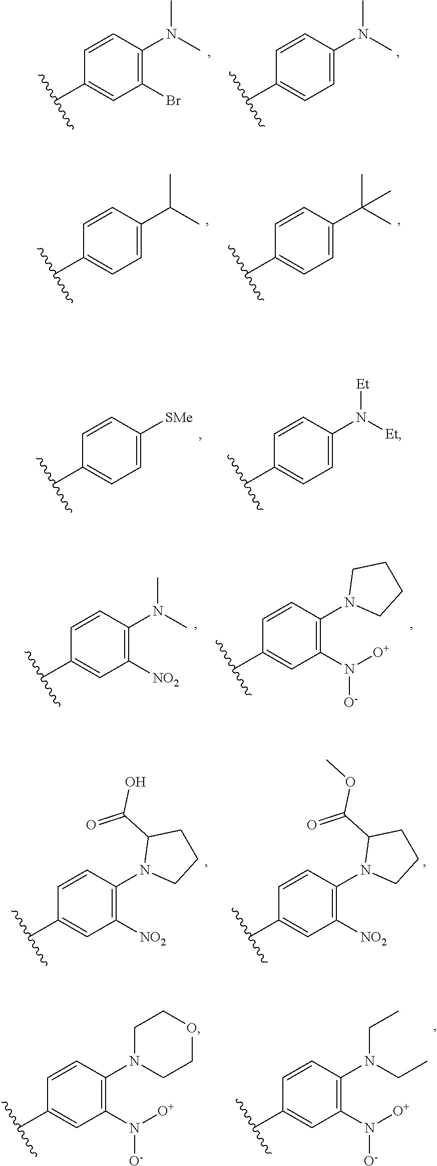

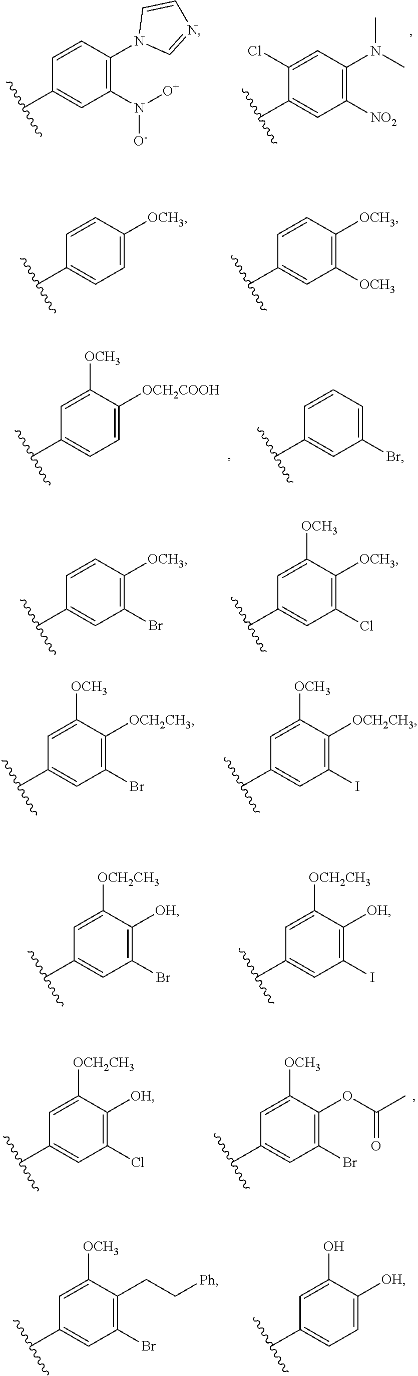

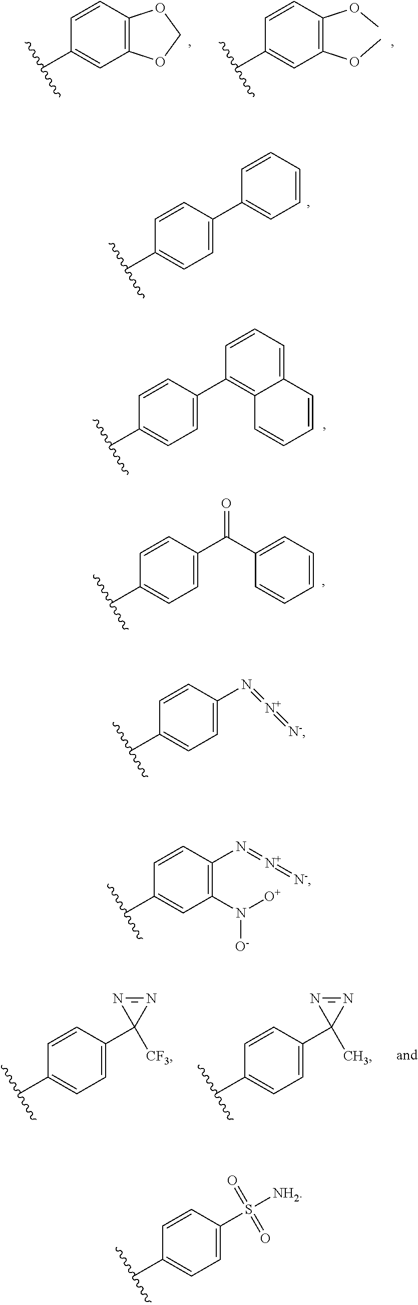

In a preferred embodiment, the active moiety is

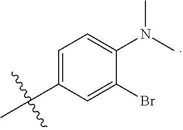 [SMILES: *c1ccc(N(C)C)c(Br)c1]

In at least one embodiment of the methods of the present application, the inhibitor is a compound selected from the group consisting of glutaminase inhibitors identified in International Application No. PCT/US2010/028688 to Cerione et al. (filed Mar. 25, 2010) (which is hereby incorporated by reference in its entirety), optionally modified to include a photoreactive moiety, if not already present; glutaminase inhibitors identified in Katt et al., Mol. Cancer Ther. 11:1269-78 (2012) (“Katt 2012”, which is hereby incorporated by reference in its entirety), optionally modified to include a photoreactive moiety, if not already present; and glutaminase inhibitors identified in International Application No. PCT/US2015/064152 to Cerione et al. (filed Dec. 5, 2015) (which is hereby incorporated by reference in its entirety), optionally modified to include a photoreactive moiety, if not already present, and optionally modified to exclude a photoreactive moiety, if present. In at least one embodiment of all aspects of the present application, the inhibitor is selected from the group consisting of Compound 968, Compound 27 of Katt 2012, Compound 17 of Katt 2012, Compound 23 of Katt 2012, Compound SU-1 of PCT/US2015/064152, Compound SU-6 of PCT/US2015/064152, Compound SU-12 of PCT/US2015/064152, Compound SU-14 of PCT/US2015/064152, Compound SU-21 of PCT/US2015/064152, and Compound SU-29 of PCT/US2015/064152.

Some aspects of the present application involve selecting a cancerous cell or cancerous tissue. In some aspects/embodiments, the cancerous cell or cancerous tissue is characterized by GLS2 overexpression and/or GLS2 hyperactivity. In some embodiments, the cancerous cell/tissue is also characterized by GLS overexpression and/or hyperactivity. Suitable cells/tissue include cells/tissue of the cancer types set forth infra. Suitable cells/tissue include cells/tissue taken or derived from the subjects set forth infra.

Some aspects of the present application involve treating a subject with a condition mediated by production of glutamate from glutamine by GLS2. In some embodiments, the condition is also mediated by production of glutamate from glutamine by GLS. As will be apparent to the skilled artisan, such conditions include those in which GLS and GLS2 are simultaneously active in the absence of a GLS2 inhibitor or dual GLS/GLS2 inhibitor, as well as conditions in which, in the absence of a GLS2 inhibitor or dual GLS/GLS2 inhibitor, GLS and GLS2 are active at different stages of the condition (for example, when a reduction in GLS activity is followed by subsequent activation of (or increase in) GLS2 activity).

In at least one embodiment of the methods of the present application, the condition mediated by production of glutamate from glutamine is a cancer. The cancer may exhibit active GLS2 glutaminase activity. Exemplary cancers include, but are not limited to, breast cancer, triple-negative breast cancer, receptor-positive breast cancer, acute myeloid leukemia, bladder cancer, bladder urothelial carcinoma, brain lower grade glioma, cervical cancer, cervical squamous cell carcinoma, radiation-resistant cervical cancer, colorectal cancer, colorectal tumor, colon adenocarcinoma, glioblastoma multiforme, head and neck cancer, head and neck squamous cell carcinoma, kidney cancer, kidney chromophobe, kidney renal papillary cell carcinoma, large B cell lymphoma, liver cancer, liver hepatocellular carcinoma, lung cancer, lung adenocarcinoma, lung squamous cell carcinoma, melanoma, non-small cell lung cancer, neuroblastoma, ovarian cancer, ovarian serous cystadenocarcinoma, pancreatic cancer, pancreatic adenocarcinoma, pancreatic ductal adenocarcinoma, paraganglial cancer, paraganglioma, prostate cancer, prostate adenocarcinoma, rectal cancer, rectal adenocarcinoma, testicular cancer, testicular germ cell tumors, thymal cancer, thymoma, thyroid cancer, thyroid carcinoma, and uterine corpus endometrial carcinoma.

In some embodiments of the methods of the present application, the cancer is characterized by moderate-to-high GLS2 expression. In some embodiments of the methods of the present application, the cancer is characterized by low GLS expression. See Table 2 for the expression patterns of GLS and GLS2 in various cancer types. In Table 2 below, “low” is defined as the majority of samples having gene expression less than the average expression level across all tissues examined. “Moderate” is defined as the majority of samples having gene expression close to the average expression level across all tissues examined. “High” is defined as the majority of samples having gene expression levels above the average expression level across all tissues examined.

TABLE 2
Expression patterns of GLS and GLS2 in various cancer types

| | | | 5 year |
|---|---|---|---|
| Cancer | GLS | GLS2 | survival (%) |
| Acute Myeloid Leukemia | Moderate | High | 65 |
| Bladder Urothelial Carcinoma | Low | High | 78 |
| Brain Lower Grade Glioma | Low | Moderate | 35 |
| Cervical Squamous cell carcinoma | Low | High | 69 |
| Colon Adenocarcinoma | High | High | 65 |
| Gliobolastoma Multiforme | Low | Low | 35 |
| Head and Neck Squamous Cell | High | Moderate | 21 |
| Carcinoma | | | |
| Kidney Chromophobe | High | High | 75 |
| Kidney Renal Clear Cell | High | Low | 75 |
| Kidney Renal Papillary Cell | High | High | 75 |
| Carcinoma | | | |
| Large B Cell Lymphoma | High | Low | 74 |
| Liver Hepatocellular Carcinoma | Low | High | 19 |
| Lung Adenocarcinoma | High | High | 20 |
| Lung Squamous Cell Carcinoma | Low | High | 20 |
| Melanoma | High | Low | 94 |
| Mesothelioma | High | Low | |
| Ocular Melanoma | High | Low | |
| Ovarian Serous | Low | High | 48 |
| Cystadenocarcinoma | | | |
| Pancreatic Adenocarcinoma | High | Moderate | 9 |
| Paraganglioma | Low | High | |
| Prostate Adenocarcinoma | Low | High | 99 |
| Receptor-positive Breast | Low | High | 91 |
| Rectal Adenocarcinoma | High | High | 69 |
| Sarcoma | High | Low | |
| Testicular Germ Cell Tumors | Low | High | 97 |
| Thymoma | Low | High | |
| Thyroid Carcinoma | High | High | 98 |
| Triple-negative Breast | High | Low | 91 |
| Uterine Carcinoma | Low | Low | 69 |
| Uterine Corpus Endometrial | Low | Moderate | 83 |
| Carcinoma | | | |

In at least some preferred embodiments of the methods of the present application, the cancer is selected from the group consisting of receptor-positive breast cancer, bladder urothelial carcinoma, brain lower grade glioma, cervical squamous cell carcinoma, liver hepatocellular carcinoma, lung squamous cell carcinoma, ovarian serous cystadenocarcinoma, paraganglioma, prostate adenocarcinoma, testicular germ cell tumors, thymoma, and uterine corpus endometrial carcinoma). In at least one embodiment of the methods of the present application, the cancer is a breast cancer. In at least one embodiment of the methods of the present application, the cancer can is a receptor-positive breast cancer or a luminal type breast cancer (e.g., a luminal type A breast cancer or a luminal type B breast cancer).

In some embodiments of the methods of the present application, the cancer is characterized by GLS2 hyperactivity. Furthermore, the cancer can be characterized by GLS2 overexpression. Additionally, the cancer may be characterized by only one of GLS2 overexpression and GLS2 hyperactivity.

In some embodiments of the methods of the present application, the cancer is characterized by GLS overexpression and/or GLS hyperactivity and characterized by GLS2 overexpression and/or GLS2 hyperactivity.

In some embodiments of the methods of the present application, the cancer is resistant to treatment with a GLS-specific inhibitor (i.e., glutaminase inhibitor that inhibits at least one GLS isoform but does not inhibit an GLS2 isoform). Such GLS-specific inhibitors include CB-839, BPTES, and BPTES-like compounds.

The methods of the present application involve contacting a cell/tissue with an inhibitor or administering an inhibitor to a subject. In at least one embodiment of the methods of the present application, the contacting or administering includes inhibiting cell proliferation, tumorigenesis, tumor growth, tumor initiation, and/or metastasis.

Administration may be performed parenterally, orally, subcutaneously, intravenously, intramuscularly, extraperitoneally, by intranasal instillation, or by application to mucous membranes.

Numerous standard references are available that describe procedures for preparing various formulations suitable for administering the compounds according to the application. Examples of potential formulations and preparations are contained, for example, in the HANDBOOK OF PHARMACEUTICAL EXCIPIENTS (American Pharmaceutical Association, current edition), PHARMACEUTICAL DOSAGE FORMS: TABLETS (Lieberman et al. eds., Marcel Dekker, Inc., pubs., current edition), and REMINGTON'S PHARMACEUTICAL SCIENCES 1553-93 (Arthur Osol ed., current edition), which are hereby incorporated by reference in their entirety.

Any pharmaceutically acceptable liquid carrier suitable for preparing solutions, suspensions, emulsions, syrups and elixirs may be employed in the composition of the application. Compounds for administration may be dissolved or suspended in a pharmaceutically acceptable liquid carrier such as water, an organic solvent, or a pharmaceutically acceptable oil or fat, or a mixture thereof. The liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like. Examples of liquid carriers suitable for oral and parenteral administration include water (particularly containing additives as above, e.g., cellulose derivatives, preferably sodium carboxymethyl cellulose solution), alcohols (including monohydric alcohols and polyhydric alcohols, e.g., glycols) or their derivatives, or oils (e.g., fractionated coconut oil and arachis oil). For parenteral administration the carrier may also be an oily ester such as ethyl oleate or isopropyl myristate.

It will be understood that the specific dose level for any particular patient will depend upon a variety of factors, including the activity of the specific compound employed, the age, body weight, general health, sex, diet time of administration, route of administration, rate of excretion, drug combination and the severity of the particular disease undergoing therapy.

In all aspects of the present application directed to methods involving contacting a sample with an inhibitor, contacting can be carried out using methods that will be apparent to the skilled artisan, and can be done in vitro, ex vivo, or in vivo.

Compounds may be delivered directly to a targeted cell/tissue/organ. Additionally and/or alternatively, the compounds may be administered to a non-targeted area along with one or more agents that facilitate migration of the compounds to (and/or uptake by) a targeted tissue, organ, or cell. As will be apparent to one of ordinary skill in the art, the compound itself can be modified to facilitate its transport to a target tissue, organ, or cell, including its transport across the blood-brain barrier; and/or to facilitate its uptake by a target cell (e.g., its transport across cell membranes).

In vivo administration can be accomplished either via systemic administration to the subject or via targeted administration to affected tissues, organs, and/or cells, as described above. Typically, the therapeutic agent (i.e., a GLS2 inhibitor or dual GLS/GLS2 inhibitor) will be administered to a patient in a vehicle that delivers the therapeutic agent(s) to the target cell, tissue, or organ. Typically, the therapeutic agent will be administered as a pharmaceutical formulation, such as those described above.

The compounds can be administered, e.g., by intravenous injection, intramuscular injection, subcutaneous injection, intraperitoneal injection, topical application, sublingual, intraarticular (in the joints), intradermal, buccal, ophthalmic (including intraocular), intranasally (including using a cannula), or by other routes. The compounds can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682, which is hereby incorporated by reference in its entirety) via a liposomal formulation (see, e.g., European Patent No. 736299, WO 99/59550, and WO 97/13500, which are hereby incorporated by reference in their entirety), via formulations described in WO 03/094886, which is hereby incorporated by reference in its entirety, or in some other form. The compounds can also be administered transdermally (i.e. via reservoir-type or matrix-type patches, microneedles, thermal portion, hypodermic needles, iontophoresis, electroporation, ultrasound or other forms of sonophoresis, jet injection, or a combination of any of the preceding methods (Prausnitz et al., Nature Reviews Drug Discovery 3:115 (2004), which is hereby incorporated by reference in its entirety). The compounds can be administered locally, for example, at the site of injury to an injured blood vessel. The compounds can be coated on a stent. The compounds can be administered using high-velocity transdermal particle injection techniques using the hydrogel particle formulation described in U.S. Patent Publication No. 20020061336, which is hereby incorporated by reference in its entirety. Additional particle formulations are described in WO 00/45792, WO 00/53160, and WO 02/19989, which are hereby incorporated by reference in their entirety. An example of a transdermal formulation containing plaster and the absorption promoter dimethylisosorbide can be found in WO 89/04179, which is hereby incorporated by reference in its entirety. WO 96/11705, which is hereby incorporated by reference in its entirety, provides formulations suitable for transdermal administration.

For use as aerosols, a compound in solution or suspension may be packaged in a pressurized aerosol container together with suitable propellants, for example, hydrocarbon propellants like propane, butane, or isobutane with conventional adjuvants. The compounds also may be administered in a non-pressurized form.

Exemplary delivery devices include, without limitation, nebulizers, atomizers, liposomes (including both active and passive drug delivery techniques) (Wang & Huang, “pH-Sensitive Immunoliposomes Mediate Target-Cell-Specific Delivery and Controlled Expression of a Foreign Gene in Mouse,” Proc. Nat'l Acad. Sci. USA 84:7851-55 (1987); Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; U.S. Pat. No. 5,059,421 to Loughrey et al.; Wolff et al., “The Use of Monoclonal Anti-Thy1 IgG1 for the Targeting of Liposomes to AKR-A Cells in Vitro and in Vivo,” Biochim. Biophys. Acta 802:259-73 (1984), each of which is hereby incorporated by reference in its entirety), transdermal patches, implants, implantable or injectable protein depot compositions, and syringes. Other delivery systems which are known to those of skill in the art can also be employed to achieve the desired delivery of the compound to the desired organ, tissue, or cells in vivo to effect this aspect of the present application.

Contacting (including in vivo administration) can be carried out as frequently as required and for a duration that is suitable to provide the desired effect. For example, contacting can be carried out once or multiple times, and in vivo administration can be carried out with a single sustained-release dosage formulation or with multiple (e.g., daily) doses.

The amount to be administered will, of course, vary depending upon the particular conditions and treatment regimen. The amount/dose required to obtain the desired effect may vary depending on the agent, formulation, cell type, culture conditions (for ex vivo embodiments), the duration for which treatment is desired, and, for in vivo embodiments, the individual to whom the agent is administered.

Effective amounts can be determined empirically by those of skill in the art. For example, this may involve assays in which varying amounts of the compound of the application are administered to cells in culture and the concentration effective for obtaining the desired result is calculated. Determination of effective amounts for in vivo administration may also involve in vitro assays in which varying doses of agent are administered to cells in culture and the concentration of agent effective for achieving the desired result is determined in order to calculate the concentration required in vivo. Effective amounts may also be based on in vivo animal studies.

The compounds can be administered alone or as an active ingredient of a pharmaceutical formulation, such as those described above. The compounds can be administered in a form where the active ingredient is substantially pure.

In the methods of the present application involving selecting a subject, the subject is preferably a human subject, but can be any animal, including a laboratory animal in the context of a clinical trial or screening or activity experiment. Thus, as can be readily appreciated by one of ordinary skill in the art, the methods of the present application are particularly suited to administration to any animal, particularly a mammal, and including, but by no means limited to, humans, domestic animals, such as feline (e.g., cats) or canine (e.g., dogs) subjects, farm animals, such as but not limited to bovine (e.g., cows), equine (e.g., horses), caprine (e.g., goats), ovine (e.g., sheep), and porcine (e.g., pigs) subjects, wild animals (whether in the wild or in a zoological garden), research animals, such as mice, rats, rabbits, guinea pigs, goats, sheep, pigs, dogs, cats, horses, cows, camels, llamas, monkeys, zebrafish etc., avian species, such as chickens, turkeys, songbirds, etc., i.e., for veterinary medical use.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

The present technology may be further illustrated by reference to the following examples.

### EXAMPLES

The examples below are intended to exemplify the practice of embodiments of the disclosure but are by no means intended to limit the scope thereof.

### Materials and Methods

### Human Cell Lines

Human breast cancer cell lines MCF7, BT-474, T-47D, MDA-MB-453, MDA-MB-231, Hs 578T, HCC38, SK-BR-3, and DU4475 were purchased from the American Type Culture Collection (ATCC) and no additional cell authentication was performed. The TSE human breast cancer cell line was supplied by Dr. Steven Abcouwer (University of Michigan). All breast cancer cells were cultured at 37° C. under a 5% CO₂ atmosphere in RPMI 1640 medium containing 2 mM glutamine (Gibco) and supplemented with 10% fetal bovine serum (FBS) (Gibco). The 293T cell line (ATCC) used for generating lentivirus particles was cultured as above but using DMEM, high glucose (Gibco).

### Animals

All experiments involving mice were carried out according to protocols approved by the Institutional Animal Care and Use Committee at Cornell University. In all cases, 6-8 week old female NOD.Cg-Prkdc_{scid} Il2rg^{tm1Wjl}/SzJ (NSG) mice (The Jackson Laboratory) were used. For xenograft experiments, a suspension of MDA-MB-453 breast cancer cells (or derivative cell lines stably expressing shRNAs) was mixed 1:1 with Matrigel Matrix (BD Biosciences) to give a final concentration of 3×10⁶ cells per 100 μl, and 3×10⁶ cells were immediately injected into each of the two flanks of 6-8 week old female NOD.Cg-Prkdc^{scid} Il2rg^{tm1Wjl}/SzJ (NSG) mice (n=3 mice per condition). Tumor sizes were measured using calipers, and estimated volumes were calculated using the formula V=(π/6)×length×width², as described previously (Tomayko et al., “Determination of Subcutaneous Tumor Size in Athymic (Nude) Mice,” Cancer Chemother Pharmacol. 24:148-154 (1989), which is hereby incorporated by reference in its entirety). For experiments using MDA-MB-453 cells stably expressing shRNAs, mice were sacrificed after 6 weeks. For 968 treatment studies, when tumors of 1-2 mm diameter were detected, the mice were randomly divided into two groups and intraperitoneal (IP) injections of 968 (10 mg/kg) solution or carrier solution were initiated and carried out three times weekly. The formulation was prepared immediately prior to injection and consisted of 70% PBS, 20% Cremophor EL, 10% DMSO, and 968 diluted from a 21 mM DMSO stock. Mice were sacrificed after 3 weeks of treatment.

### Cell Lysis and Western Blot Analysis

Cells, or fractionated mitochondria and nuclei, were lysed with ice-cold lysis buffer (50 mM HEPES pH 8.0, 150 mM NaCl, 25 mM NaF, 1% (v/v) Triton X-100, 1 mM MgCl₂, 50 mM β-glycerophosphate, 30 μg/ml leupeptin, 5 μg/ml aprotinin), and insoluble debris cleared by centrifugation at 4° C. Protein concentrations were determined by Bradford assay (Bio-Rad), and lysate was then boiled for 10 min in reducing SDS-sample buffer. Lysate proteins (20 μg total protein/lane, except for the cell fractionation experiment in which 5 μg total protein/lane was used) were then resolved on Novex 12% Tris-Glycine Mini Gels (Thermo Fisher Scientific) and transferred to PVDF membrane (PerkinElmer). Membranes were blocked in 7% BSA in Tris-Buffered Saline plus 0.05% TWEEN 20 (TBST) for 1 hour at room temperature and probed overnight at 4° C. in primary antibody solution in TBST (see Table 3 for antibody dilutions). They were then washed with TBST and incubated in TBST containing 25% (v/v) non-fat dry milk powder and anti-Rabbit or anti-Mouse secondary antibody (1:2500) for 1 hour. Finally, membranes were washed in TBST, and imaged using Western Lightning Plus-ECL (PerkinElmer) and HyBlot ES autoradiography film (Denville Scientific Inc.).

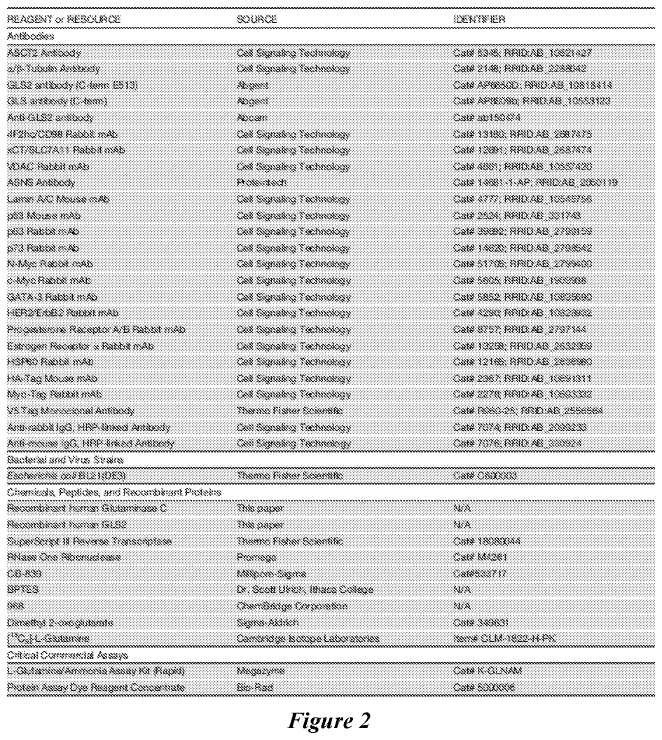

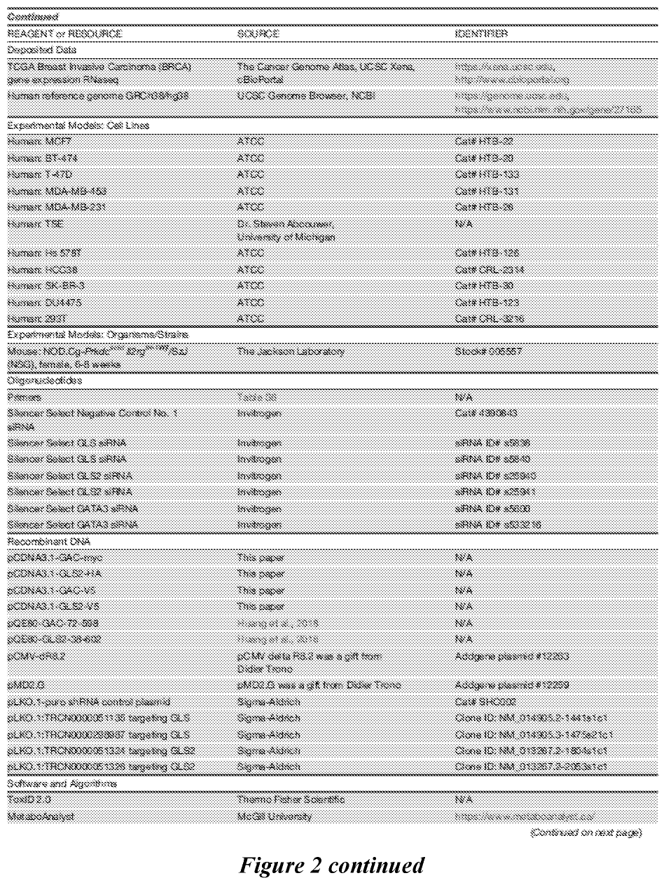

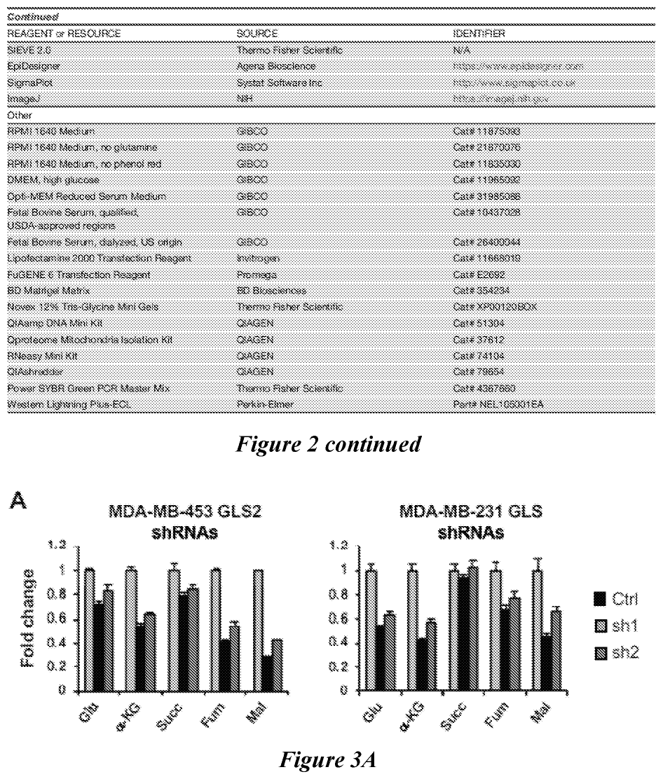

TABLE 3
Antibody dilutions used for western blot analysis related to
STAR Methods (see FIG. 2 for Star Method Resource Table).

| Antibody | Dilution |
|---|---|
| ASCT2 Antibody | 1:2000 |
| α/β-Tubulin Antibody | 1:5000 |
| GLS antibody (C-term) | 1:10000 |
| Anti-GLS2 antibody (C-term) | 1:8000 |
| 4F2hc/CD98 (SLC3A2) antibody | 1:2000 |
| xCT/SLC7A11 antibody | 1:2000 |
| VDAC Rabbit mAb | 1:4000 |
| ASNS Antibody | 1:4000 |
| Lamin A/C Mouse mAb | 1:5000 |
| p53 Mouse mAb | 1:4000 |
| p63 Rabbit mAb | 1:5000 |
| p73 Rabbit mAb | 1:5000 |
| N-Myc Rabbit mAb | 1:5000 |
| c-Myc Rabbit mAb | 1:2000 |
| GATA-3 Rabbit mAb | 1:32000 |
| HER2/ErbB2 Rabbit mAb | 1:4000 |
| Progesterone Receptor A/B Rabbit mAb | 1:4000 |
| Estrogen Receptor α Rabbit mAb | 1:4000 |
| V5 Tag Monoclonal Antibody | 1:8000 |

### Breast Tumor Tissue Microarray

Tissue microarray BRC961 (US Biomax) was probed as follows, using the anti-GLS2 antibody (Abgent, AP6650D). Reagents were from Vector Laboratories VECTASTAIN Elite ABC HRP kit (Cat #PK-6200), Avidin/Biotin blocking kit (Cat #SP-2001), and ImmPACT DAB Peroxidase (HRP) Substrate (Cat #SK-4105). Deparaffinization was carried out by heating the slide to 60° C. for 20 min and then immersing the slide in mixed xylenes (2×10 min), 100% ethanol, 95% ethanol, 70% ethanol (5 min each) and finally H₂O (2×5 min). The antigen retrieval step involved immersing the slide in 10 mM sodium citrate buffer, pH 6.0 at ˜95° C. for 15 min and then cooling at room temperature for 20 min. To remove endogenous peroxidase the slide was washed with H₂O (2×5 min) and then incubated in 3% H₂O₂ in H₂O for 10 min. The slide was then washed in H₂O for 5 min followed by PBS for 5 min. Blocking was carried out at room temperature using horse serum (20 min), PBS rinse, avidin solution (15 min), PBS rinse, biotin solution (15 min), PBS rinse, followed by PBS washes (2×5 min). The slide was then incubated overnight at 4° C. with the anti-GLS2 primary antibody (1:100 in horse serum). Next, the slide was washed with PBS (4×5 min), incubated for 30 min at room temperature with biotinylated universal secondary antibody, and washed again with PBS (4×5 min). Then, the slide was incubated for 30 min at room temperature with VECTASTAIN ABC reagent and washed again with PBS (4×5 min). To develop, the slide was incubated with diluted ImmPACT DAB chromogen for ˜2 min and then washed in H₂O (2×5 min). Finally, the stained slide was dehydrated by immersing in 70% ethanol, 95% ethanol, 100% ethanol (5 min each) and mixed xylenes (2×5 min), mounted using Permount mounting medium (Fisher) and sealed. Signal intensity was quantified using ImageJ. Receptor staining intensity data were from US Biomax.

RNA Isolation and Quantitative Real-Time PCR (qRT-PCR)

Total RNA was extracted from cells using the RNeasy Mini Kit (Qiagen) and QIAshredder (Qiagen), and cDNAs were prepared using SuperScript III Reverse Transcriptase (Thermo Fisher Scientific). RT-PCR was carried out using the 7500 fast real-time PCR system (Applied Biosystems), using appropriate primers (Table 4) with cDNA as the template. In all cases, 18S rRNA served as the endogenous control. All primer sequences were obtained from PrimerBank (https://pga.mgh.harvard.edu/primerbank/), and primers were synthesized by Integrated DNA Technologies. Reactions were carried out using Power SYBR Green PCR Mix (Thermo Fisher Scientific).

TABLE 4
List of primers related to STAR Methods

| Name | Sequence | Sequence ID |
|---|---|---|
| 18S rRNA FWD | CGGCGACGACCCATTCGAAC | SEQ ID NO: 4 |
| 18S rRNA REV | GAATCGAACCCTGATTCCCCGTC | SEQ ID NO: 5 |
| GLS FWD | TCTACAGGATTGCGAACGTCT | SEQ ID NO: 6 |
| GLS REV | CTTTGTCTAGCATGACACCATCT | SEQ ID NO: 7 |
| GLS2 FWD | GCCTGGGTGATTTGCTCTTTT | SEQ ID NO: 8 |
| GLS2 REV | CCTTTAGTGCAGTGGTGAACTT | SEQ ID NO: 9 |
| SLC7A11 FWD | GGTCCATTACCAGCTTTTGTACG | SEQ ID NO: 10 |
| SLC7A11 REV | AATGTAGCGTCCAAATGCCAG | SEQ ID NO: 11 |
| GLS2_000_10F | AGGAAGAGAGGGAGGTAGGAGGTTGTTTTATAT | SEQ ID NO: 12 |
| | TT | |
| GLS2_000_T7R | CAGTAATACGACTCACTATAGGGAGAAGGCTCT | SEQ ID NO: 13 |
| | TACCCCCACTCCCACTATAATTC | |
| GLS2_001_10F | AGGAAGAGAGATTATAGTGGGAGTGGGGGTAA | SEQ ID NO: 14 |
| | GT | |
| GLS2_001_T7R | CAGTAATACGACTCACTATAGGGAGAAGGCTTC | SEQ ID NO: 15 |
| | CTAAAAATACCCCTAACCCTAAA | |
| GLS2_002_10F | AGGAAGAGAGGGGTTTGTTTTTTTAAAGTTGGTT | SEQ ID NO: 16 |
| | T | |
| GLS2_002_T7R | CAGTAATACGACTCACTATAGGGAGAAGGCTAC | SEQ ID NO: 17 |
| | TCTCATTAAACAACCTTACCCTTT | |
| GLS2_003_10F | AGGAAGAGAGGAGTTGGAATGGATAATGTTTAG | SEQ ID NO: 18 |
| | GTT | |
| GLS2_003_T7R | CAGTAATACGACTCACTATAGGGAGAAGGCTCC | SEQ ID NO: 19 |
| | CAATACCTCCCTAAAATACTAATC | |
| GLS2_004_10F | AGGAAGAGAGAAAGGGTAAGGTTGTTTAATGA | SEQ ID NO: 20 |
| | GAGT | |
| GLS2_004_T7R | CAGTAATACGACTCACTATAGGGAGAAGGCTCA | SEQ ID NO: 21 |
| | CAAACTAAAACCCAAACTTCCTA | |
| GLS2_005_10F | AGGAAGAGAGTAAGGTTTTTGGGATAGGGTAGG | SEQ ID NO: 22 |
| GLS2_005_T7R | CAGTAATACGACTCACTATAGGGAGAAGGCTAC | SEQ ID NO: 23 |
| | ACAAACCCCAATACACCTAAAC | |
| GLS2_006_10F | AGGAAGAGAGTGAAGGTTTTGTAGAAGGTTTTG | SEQ ID NO: 24 |
| | AG | |
| GLS2_006_T7R | CAGTAATACGACTCACTATAGGGAGAAGGCTCC | SEQ ID NO: 25 |
| | ACAATAAAAATAAAAACAAACCC | |
| GLS2_007_10F | AGGAAGAGAGTGATTGGATTTGGGTATTGTTTA | SEQ ID NO: 26 |
| | TT | |
| GLS2_007_T7R | CAGTAATACGACTCACTATAGGGAGAAGGCTCT | SEQ ID NO: 27 |
| | CAAAACCTTCTACAAAACCTTCA | |
| GLS2_008_10F | AGGAAGAGAGGTTTAATGTTTTTTTGAGGTGTTG | SEQ ID NO: 28 |
| | G | |
| GLS2_008_T7R | CAGTAATACGACTCACTATAGGGAGAAGGCTAA | SEQ ID NO: 29 |
| | AATAAACAATACCCAAATCCAATC | |
| GLS2_009_10F | AGGAAGAGAGTAAATAAATGGTAGAGGGATAT | SEQ ID NO: 30 |
| | AAATTGG | |
| GLS2_009_T7R | CAGTAATACGACTCACTATAGGGAGAAGGCTTT | SEQ ID NO: 31 |
| | CAAAAAATCACTCAAAAAACCTC | |

### Genomic DNA Isolation and DNA Methylation Analysis

High molecular weight genomic DNA was isolated from breast cancer cells using QIAamp DNA Mini Kit (Qiagen), and contaminating RNA was digested using RNase One Ribonuclease (Promega) followed by re-purification of DNA, elution with water, and adjustment of DNA concentration to 50 ng/μl. To assess DNA purity, UV/visible absorption spectra were measured, and for all samples the A260/280 ratio was >1.7 and the A260/230 ratio was >2.0. Samples were then submitted to the Epigenomics Core at Weill Cornell Medical College, where bisulfite conversion was carried out followed by DNA methylation analysis using Mass ARRAY EpiTYPER1.2 Suite (Agena Bioscience). The sequence of the CpG island in the GLS2 gene promoter was obtained from human reference genome GRCh38/hg38 using the UCSC Genome Browser (https://genome.ucsc.edu). Primers for DNA methylation analysis were designed using EpiDesigner (Agena Bioscience) and synthesized by Integrated DNA Technologies (Table 4). Relative methylation ratios at CpG sites are presented as a heatmap, generated using MORPHEUS (https://software.broadinstitute.org/morpheus).

### Immunofluorescence

Cells were fixed with 3.7% formaldehyde in PBS for 20 min and then permeabilized with PBS containing 0.1% Triton X-100 (v/v) for 20 min. Samples were blocked with 10% BSA (w/v) in PBS for 1 hour, rinsed with PBS and then incubated with primary antibody in PBS containing 5% BSA (w/v) for 2 hours. Samples were washed 3 times with PBS, and then Texas Red- or Oregon Green 488-conjugated secondary antibody diluted 1:400 in PBS containing 5% BSA (w/v), along with DAPI counterstain, was added for 1 hour. Samples were washed 3 times with PBS, mounting medium was applied, and slides were sealed with a cover slip prior to imaging with a ZEISS Axioscope.

### Cell Fractionation and Mitochondrial Isolation

Breast cancer cells were fractionated into cytosolic, mitochondrial, and nuclear components via partial lysis and centrifugation using the Qproteome Mitochondria Isolation Kit (Qiagen). For all samples, a portion of the starting material (i.e. whole cells) was retained for comparison with the isolated fractions. When cellular fractions were to be analyzed only by western blot, a single 10 cm dish of exponentially growing cells was used. When mitochondria were isolated for glutaminase assays, two 15 cm dishes of exponentially growing cells were used.

### Mitochondrial Glutaminase Activity Assays

A two-reaction protocol was used to measure mitochondrial glutaminase activity. Mitochondria (5 pg total protein) were added to 105 μl of Reaction Mix 1 (20 mM glutamine, 0.2 mM EDTA, 50 mM Tris-acetate pH 8.6), supplemented with 10 μM BPTES when appropriate, and samples were incubated at 37° C. for 40 min. The reaction was then quenched by addition of 10 μl of 2.4 M HCl, and samples placed on ice. Next, 20 μl of quenched Reaction Mix 1 was added to 200 μl of Reaction Mix 2 (1 unit bovine liver glutamate dehydrogenase (Sigma-Aldrich), 80 mM Tri-HCl pH 9.4, 200 mM hydrazine, 0.25 mM ADP, 2 mM NAD) and samples were incubated for 1 hour at room temperature. The absorbance at 340 nm was then measured against a matched sample in which heat-inactivated mitochondria (immersed in boiling water for 5 minutes) were used. A standard curve was prepared using given concentrations of glutamate in Reaction Mix 2, allowing the amount of glutamate produced in Reaction 1 to be determined.

### Real-Time Recombinant Glutaminase Activity Assays

Real-time monitoring of glutaminase activity through NADH production was performed on a Cary Eclipse fluorescence spectrometer. The excitation and emission wavelengths were set at 340 nm and 460 nm, respectively. To a 1 ml cuvette, 900 μl of assay buffer was added, followed by 10 μl of GDH, 40 μl of 50 mM NAD and 20 μl of either DMSO or various dilutions of 968. Then, 100 μl of either 100 nM GAC or 500 nM GLS2 was added to this mixture and the fluorescence emission was monitored in real time. After 30 s, 200 μl of a mixture of glutamine and K₂HPO₄ was added such that the final concentrations of K₂HPO₄ and glutamine were 100 mM and 20 mM, respectively. The initial velocity of glutamine hydrolysis was obtained from the slopes of the linear portion of the kinetic curve.

### Glutamine Consumption and Glutamate Release Assays

To 6-well plates containing 2 ml phenol red-free culture medium/well, 2×10⁵ cells/well were added and incubated overnight to attach. Wells were then rinsed twice with serum-free, phenol red-free culture medium, and 2 ml/well fresh serum-free/phenol red-free medium (containing 2 mM glutamine) was added, followed by incubation for 19 hours. As a negative control, wells containing culture medium only were used. Medium was then collected, cellular debris removed by centrifugation at 4° C., and the supernatant retained and stored on ice. Meanwhile, cells attached to the wells were lysed and total protein was quantified using the Bradford assay. Glutamine concentrations were determined using the L-Glutamine/Ammonia Assay Kit (Rapid) (Megazyme) following the manufacturer's instructions. Briefly, 50 μl sample was mixed with 100 μl Assay Buffer 1 and 10 μl Glutaminase Suspension and incubated at room temperature for 5 min. For all reactions, a blank containing 50 μl H₂O was run in parallel. Then, 150 μl. Assay Buffer 2, 100 μl NADPH Solution, and H₂O to bring the final volume to 1160 μl was added, followed by incubation at room temperature for 4 min. Absorbance A₁ was then measured at 340 nm. Next, 10 μl. Glutamate Dehydrogenase Suspension was added, samples were mixed and incubated at room temperature for 5 min, and absorbance A₂ was measured at 340 nm. Sample concentrations of glutamine were calculated using the extinction coefficient of NADPH at 340 nm. Changes in sample glutamine concentrations were measured relative to the culture medium samples which had been incubated in cell-free wells. To measure glutamate levels in culture medium, samples were analyzed by Reaction 2 described above for the mitochondrial glutaminase activity assays.

### Cell Proliferation Assays

Culture medium was added to 12-well plates (1 ml/well) and wells were seeded with cells at Day 0 as follows. MCF7, T-47D, BT-474, HCC38 cells: 2×10W cells/well. MDA-MB-453 and MDA-MB-231 cells: 1×10⁴ cells/well. TSE and Hs 578T cells: 0.3×10⁴ cells/well. After 16 hours, culture medium was replaced with fresh medium supplemented with appropriate concentrations of inhibitors and was subsequently replaced every 48 hours. On Day 6 cells were trypsinized and suspended in an appropriate volume of culture medium, and the total number of cells per well was determined using a hemocytometer or a TC20 Automated Cell Counter (Bio-Rad).

### DNA Constructs for Expressing GLS and GLS2

Vectors for expressing human GAC or GLS2 in breast cancer cell lines were based on pCDNA3.1/V5-His TOPO (Thermo Fisher Scientific), with the appropriate gene sub-cloned in and the tag switched to HA-tag or myc-tag for immunofluorescence experiments. Vectors pQE80-GAC-72-598 and pQE80-GLS2-38-602, for expressing the processed forms of human GAC (residues 72 to 598) or GLS2 (residues 38 to 602) in E. coli, were described in Huang et al., “Characterization of the Interactions of Potent Allosteric Inhibitors with Glutaminase C, a Key Enzyme in Cancer Cell Glutamine Metabolism,” J. Biol. Chem. 293:3535-3545 (2018), which is hereby incorporated by reference in its entirety.

### Expression and Purification of Recombinant GAC and GLS2

Recombinant GAC and GLS2 were expressed in E. coli as described in Huang et al., “Characterization of the Interactions of Potent Allosteric Inhibitors with Glutaminase C, a Key Enzyme in Cancer Cell Glutamine Metabolism,” J. Biol. Chem. 293:3535-3545 (2018), which is hereby incorporated by reference in its entirety. Briefly, E. coli strain BL21(DE3) (Thermo Fisher Scientific) were transformed with vector pQE80-GAC-72-598 or with pQE80-GLS2-38-602 to express the processed forms of GAC and GLS2, respectively. For both constructs, expression was induced with 0.3 mM IPTG for 20 hours at 18° C., and cells were then harvested by centrifugation and lysed by sonication in binding buffer (500 mM NaCl, 50 mM Tris-HCl pH 8.5, 10 mM imidazole, 5 mM β-mercaptoethanol, 1 mM benzamidine chloride). The lysate was centrifuged, and the supernatant applied to a Ni-NTA column which was then washed with 100 column volumes of binding buffer followed by 10 volumes of wash buffer (500 mM NaCl, 50 mM Tris-HCl pH 8.5, 40 mM imidazole, 5 mM β-mercaptoethanol, 1 mM benzamidine chloride). Protein was eluted with 5 column volumes of elution buffer (500 mM NaCl, 300 mM imidazole-HCl pH 7.5, 5 mM β-mercaptoethanol, 1 mM benzamidine chloride). The eluate was centrifugally concentrated and then further purified by FPLC using a HiLoad 16/600 Superdex 200 column (GE Healthcare) with 150 mM NaCl, 5 mM Tris-HCl pH 7.5.

Transfection of Breast Cancer Cells with DNA Constructs

For 60 mm dish format. 0.2 ml Opti-MEM (Gibco) containing 1.5 pg of the appropriate DNA construct, along with 0.2 ml Opti-MEM containing 12 μl Lipofectamine 2000 (Invitrogen), were separately incubated at room temperature for 5 min. The two solutions were combined and incubated for an additional 20 min, mixed with 1.6 ml culture medium and added to cells. After 5 hours incubation at 37° C. the transfection mixture was replaced with fresh culture medium, and cells were then incubated for an additional 48 hours to allow for ectopic expression of GLS or GLS2. To select for cells stably expressing the DNA construct, culture medium supplemented with 500 μg/ml G-418 disulfate (Research Products International) was added and replaced every 2 days for 2-3 weeks until isolated colonies ˜2 mm in diameter were present. Individual colonies were transferred to a 12-well plate (1 colony per well) using sterile blotting paper soaked in trypsin solution and were then cultured in medium supplemented with 250 μg/ml G-418 disulfate. All colonies were screened by western blot for ectopic expression, and positive clones were maintained in medium supplemented with 250 μg/ml G-418 disulfate.

Genetic Knockdowns Using siRNAs

Transient knockdowns of GATA3, GLS, and GLS2 were achieved using two rounds of transfection with Silencer Select pre-designed siRNAs (Invitrogen). For 60 mm dish format, 0.3 ml Opti-MEM (Gibco) containing 100 nM of the appropriate siRNA (to give a final siRNA concentration of 10 nM when diluted as below), along with 0.3 ml Opti-MEM containing 12 μl Lipofectamine 2000 (Invitrogen), were incubated separately at room temperature for 5 min. The two solutions were then combined and incubated for an additional 20 min, mixed with 2.4 ml culture medium, and added to cells. After 5 hours incubation at 37° C. the transfection mixture was replaced with fresh culture medium. For all knockdowns, two independent siRNAs were used, along with a negative control siRNA.

Genetic Knockdowns Using shRNAs

The MISSION RNAi system (Sigma-Aldrich) was used for shRNA-mediated knockdown of GLS and GLS2. Lentivirus particles for each shRNA construct were generated using exponentially-growing 293T cells (ATCC) as follows. For 10 cm dish format, 570 μl DMEM was mixed with 33 μl FuGENE 6 (Promega) and incubated at room temperature for 5 min. Plasmids pLKO.1-shRNA (5 μg), pCMV-dR8.2 (packaging vector) (5 μg), and pMD2.G (envelope vector) (1 gg) were then added to the solution, incubated for an additional 15 min, mixed with 8 ml culture medium and added to cells. Cells were incubated at 37° C. overnight and the transfection medium was then replaced with fresh culture medium, followed by an additional 24 hours incubation to allow for production of virus particles. Virus-containing medium was then collected, and cellular debris removed by centrifugation. To transduce breast cancer cells, virus-containing supernatant was diluted 1:12 in fresh culture medium, and 6 μg/ml polybrene was added before applying to cells. After 6 hours incubation at 37° C. the transduction medium was replaced with fresh culture medium, and cells were incubated for an additional 48 hours before knockdowns were validated. For both GLS and GLS2 knockdowns, two independent shRNA constructs were used (see Table 5), and for all experiments the effects of knockdown were compared with those of a control shRNA. To select for stable expression of the constructs, cells were cultured in medium containing 0.5 μg/ml puromycin.

TABLE 5
List of shRNA Constructs

| | Sigma-Aldrich | | |
|---|---|---|---|
| Name | Product No. | Target Sequence | Sequence ID |
| GLS shRNA 1 | TRCN0000051135 | GCACAGACATGGTTGGTATAT | SEQ ID NO: 32 |
| GLS shRNA 2 | TRCN0000298987 | GCACAGACATGGTTGGTATAT | SEQ ID NO: 33 |
| GLS2 shRNA 1 | TRCN0000051324 | GCCATGGATATGGAACAGAAA | SEQ ID NO: 34 |
| GLS2 shRNA 2 | TRCN0000051326 | GCCCTGTCCAAAGAGAACTTA | SEQ ID NO: 35 |

Data from the Cancer Genome Atlas

Gene expression data (RNA-Seq V2, RSEM) from TCGA invasive breast cancer dataset (Koboldt et al., “Comprehensive Molecular Portraits of Human Breast Tumours,” Nature 490:61-70 (2012), which is hereby incorporated bt reference in its entirety) were accessed using UCSC Xena (https://xena.ucsc.edu) or cBioPortal (www.cbioportal.org). Breast tumor subtype calls made by UCSC Xena were based on RNA-Seq data. Outlier readings are not shown on box and whisker plots but are included in calculation of the mean. Copy-number analysis data were accessed using cBioPortal.

### Metabolite Extraction

The procedures for metabolite extraction from cultured cells are described in previous studies (Cluntun et al., “The Rate of Glycolysis Quantitatively Mediates Specific Histone Acetylation Sites,” Cancer Metab. 3:10 (2015); Liu et al., “A Strategy for Sensitive, Large Scale Quantitative Metabolomics,” J. Vis. Exp. e51358-e51358 (2014), which are hereby incorporated by reference in their entirety). Briefly, adherent cells were grown in 6-well plates in biological triplicate to 80% confluence, medium was rapidly aspirated and cells were washed with cold PBS on ice. Then, 1 ml of extraction solvent (80% methanol/water) cooled to −80° C. was added to each well, and the dishes were transferred to −80° C. for 15 min. Cells were then scraped into the extraction solvent on dry ice. All metabolite extracts were centrifuged at 20,000×g at 4° C. for 10 min. Finally, the solvent in each sample was evaporated in a Speed Vacuum. The cell extracts were dissolved in 15 μl water and 15 μl methanol/acetonitrile (1:1 v/v) (LC-MS optima grade, Thermo Fisher Scientific). Samples were centrifuged at 20,000×g for 10 min at 4° C. and the supernatants were transferred to Liquid Chromatography (LC) vials. The injection volume for polar metabolite analysis was 8 μl.

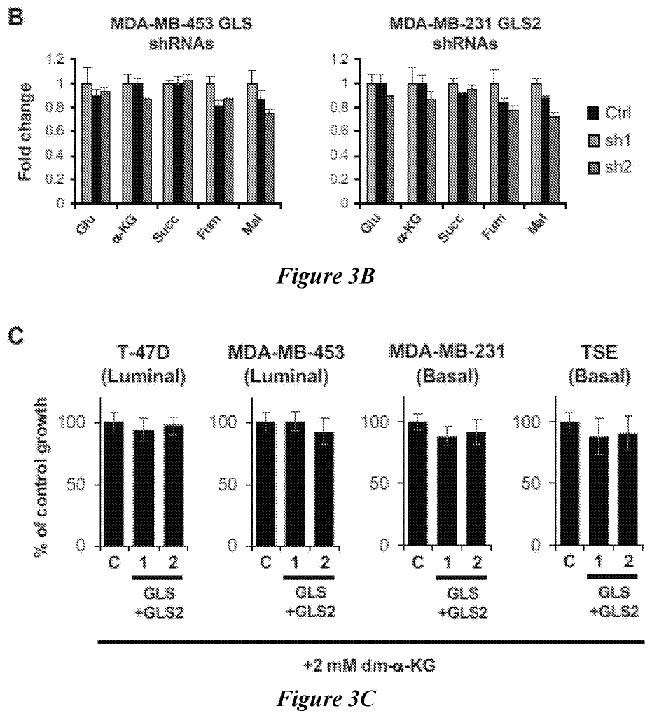

For metabolite abundances in FIGS. 3A-B a slightly modified protocol was used, as follows. The cell extracts were dissolved in 50 μl water (LC-MS optima grade, Thermo Fisher Scientific) and sonicated to ensure analytes were completely dissolved. Samples were centrifuged at 18,000×g for 30 min at 4° C. and the supernatants were transferred to Liquid Chromatography (LC) vials. The injection volume for polar metabolite analysis was 1 μl.

### [U-¹³]-Glutamine Labeling

Cells were grown to 80% confluence in 6-well plates with standard culture medium and washed with sterile PBS. Then, culture medium in which glutamine was replaced by [¹³C₅]-L-glutamine (Cambridge Isotope Laboratories), supplemented with dialyzed FBS (Gibco) and appropriate concentrations of inhibitors was added (1.5 ml/well). At the appropriate time-point, metabolites were extracted as described above.

### Liquid Chromatography

A hydrophilic interaction liquid chromatography method (HILIC) with an Xbridge amide column (100×2.1 mm, 3.5 μm) (Waters) was employed on a Dionex (Ultimate 3000 UHPLC) for compound separation and detection at room temperature. The mobile phase A was 20 mM ammonium acetate and 15 mM ammonium hydroxide in water with 3% acetonitrile, pH 9.0, and the mobile phase B was acetonitrile. The linear gradient was as follows: 0 min, 85% B; 1.5 min, 85% B, 5.5 min, 35% B; 10 min, 35% B, 10.5 min, 35% B, 14.5 min, 35% B, 15 min, 85% B, and 20 min, 85% B. The flow rate was 0.15 ml/min from 0 to 10 min and 15 to 20 min, and 0.3 ml/min from 10.5 to 14.5 min. All solvents were LCMS grade and purchased from Thermo Fisher Scientific.

For metabolite abundances in FIGS. 3A-B a slightly modified protocol was used, as follows. A hydrophilic interaction liquid chromatography method (HILIC) with an ZORBAX HILIC Plus column (150×2.1 mm, 1.8 μm) (Agilent) was employed on a Dionex (Ultimate 3000 UHPLC) for compound separation and detection at room temperature. The mobile phase water with 0.1% formic acid was mobile phase A and acetonitrile with 0.1% formic acid was mobile phase B. The linear gradient was as follows: 0 min, 95% B; 1.5 min, 95% B, 15.5 min, 50% B; 16.5 min, 10% B, 18.5 min, 10% B, 18.6 min, 95% B, 21 min, 95% B, and total flow rate was 0.5 ml/min. All solvents were LCMS grade and purchased from Thermo Fisher Scientific.

### Mass Spectrometry

The Q Exactive MS (Thermo Scientific) is equipped with a heated electrospray ionization probe (HESI), and the relevant parameters are as listed: evaporation temperature, 120° C.; sheath gas, 30; auxiliary gas, 10; sweep gas, 3; spray voltage, 3.6 kV for positive mode and 2.5 kV for negative mode. Capillary temperature was set at 320° C., and S-lens was 55. A full scan range from 60 to 900 (m/z) was used. The resolution was set at 70,000. The maximum injection time was 200 ms. Automated gain control (AGC) was targeted at 3,000,000 ions.

For metabolite abundances in FIGS. 3A-B a slightly modified protocol was used, as follows. The Q Exactive HF (Thermo Scientific) is equipped with a heated electrospray ionization probe (HESI), and the relevant parameters are as listed: evaporation temperature, 120° C.; sheath gas, 60; auxiliary gas, 20; sweep gas, 1; spray voltage, 3.0 kV for negative mode. Capillary temperature was set at 380° C., and S-lens was 50. A full scan range from 80 to 300 (m/z) was used. The resolution was set at 240,000. The maximum injection time was 500 ms. Automated gain control (AGC) was targeted at 3,000,000 ions.

### Metabolomics and Data Analysis

Raw data collected from LC-Q Exactive MS were processed on Sieve 2.0 (Thermo Scientific) and ToxID 2.0 (Thermo Scientific). Peak alignment and detection were performed according to the protocol described by Thermo Scientific. For targeted metabolite analysis, the method ‘peak alignment and frame extraction’ was applied. An input file of theoretical m/z and detected retention time of 204 known metabolites was used for targeted metabolites analysis with data collected in positive mode, while a separate input file of 278 metabolites was used for negative mode. M/Z width was set at 10 ppm. The output file including detected m/z and relative intensity in different samples was obtained after data processing. The quantity of the metabolite fraction analyzed was adjusted to the corresponding protein concentration and cell count upon processing a parallel 6-well plate. Quantitation and statistics were calculated and visualized with Microsoft Excel, MORPHEUS and MetaboAnalyst online software.

### Example 1—Luminal Breast Cancers Use Glutamine Anaplerosis but Resist GLS Inhibitors

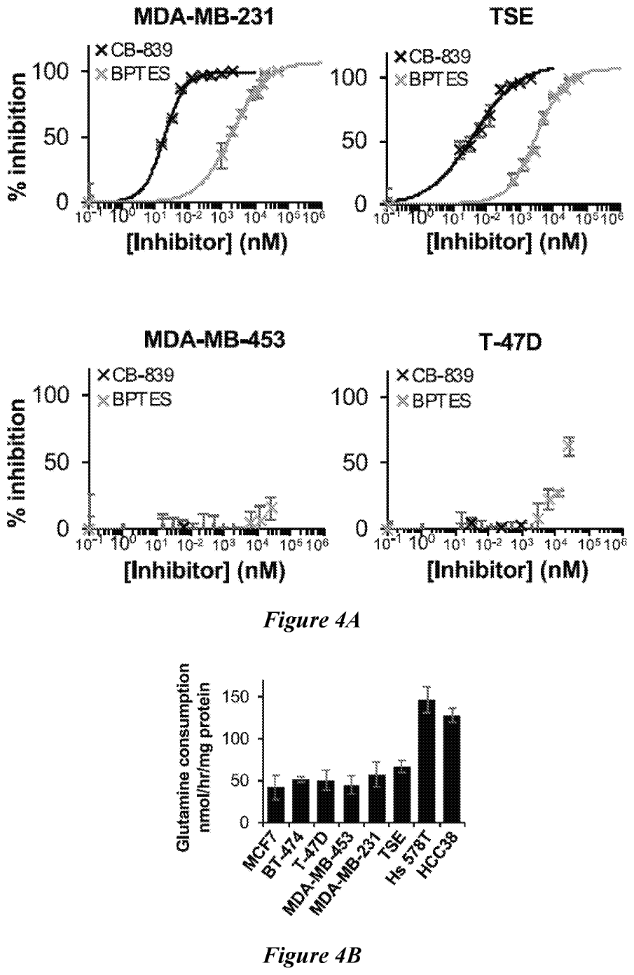

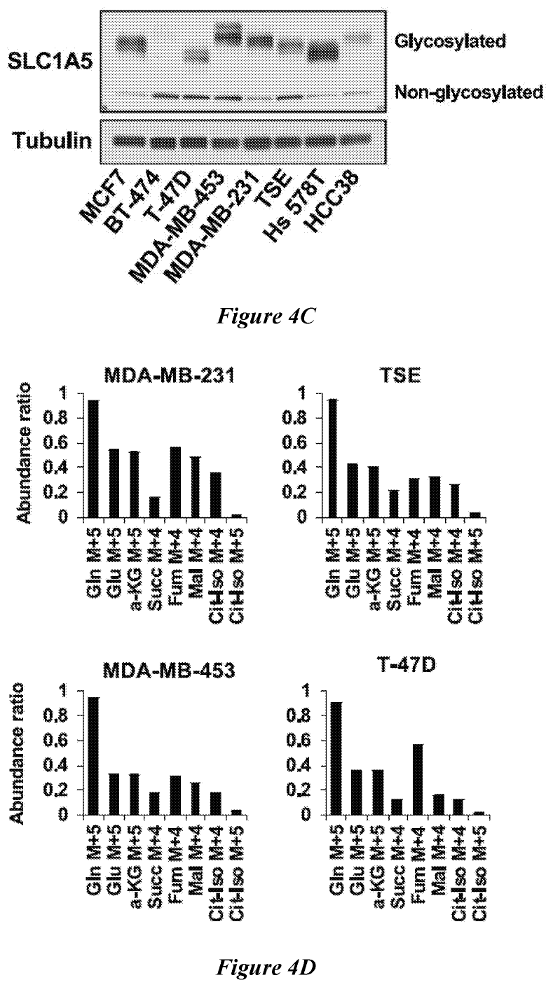

The most extensively studied inhibitors of GLS are based on the BPTES molecular scaffold, with the potent analog CB-839 currently in clinical trials for a number of malignancies. CB-839 was originally reported to be effective against triple-negative breast cancer (TNBC) cells (Gross et al., “Antitumor Activity of the Glutaminase Inhibitor CB-839 in Triple-negative Breast Cancer,” Mol. Cancer Ther. 13:890-901 (2014), which is hereby incorporated by reference in its entirety), which are characterized by low expression of the receptors ER, PR, and HER2. Across a collection of breast cancer cell lines, it was observed that basal-subtype cells respond to BPTES or CB-839 treatment, whereas luminal-subtype cells resist these inhibitors, regardless of their specific receptor status (FIG. 4A, Table 6, and Table 7). It was previously reported the same selectivity profile for the related inhibitor UPGL00004, indicating that this entire class of molecules is ineffective against luminal-subtype breast cancers (Huang et al., “Characterization of the Interactions of Potent Allosteric Inhibitors with Glutaminase C, a Key Enzyme in Cancer Cell Glutamine Metabolism,” J. Biol. Chem. 293:3535-3545 (2018), which is hereby incorporated by reference in its entirety).

TABLE 6
Description of breast cancer cell lines.

| | Reported | Reported receptor status | | | |
|---|---|---|---|---|---|
| Cell line | subtype | ER | PR | HER2 | Notes |
| MCF7 | Luminal | + | + | − | |
| | A | | | | |
| BT-474 | Luminal | + | + | + | ERBB2 gene amplified, but inverse |
| | B | | | | correlation to HER2+ molecular subtype. |
| T-47D | Luminal | + | + | − | |
| | A | | | | |
| MDA-MB- | Luminal | − | − | +/− | ERBB2 gene amplified but not |
| 453 | (LAR) | | | | overexpressed. Luminal androgen |
| | | | | | receptor (LAR) triple-negative breast |
| | | | | | cancer. |
| MDA-MB- | Basal | − | − | − | Basal B and Claudin-low subgroups. |
| 231 | | | | | |
| TSE | Basal | − | − | − | Triple-negative with strong basal markers. |
| Hs 578T | Basal | − | − | − | Basal B and Claudin-low subgroups. |
| HCC38 | Basal | − | − | − | Basal B and Claudin-low subgroups. |
| DU4475 | Basal | − | − | − | Derived from a metastatic lesion of the |
| | | | | | breast tumor located at the skin. |

TABLE 7
EC₅₀ values for BPTES and CB-839 derived
from 6-day cell proliferation assay.

| | BPTES | CB-839 |
|---|---|---|
| Cell line | EC₅₀/μM | EC₅₀/nM |
| MCF7 | ~20 | >1000 |
| BT-474 | ~20 | >1000 |
| T-47D | ~20 | >1000 |
| MDA-MB-453 | ~20 | >1000 |
| MDA-MB-231 | 2.1 | 19 |
| TSE | 3.1 | 41 |
| Hs 578T | 3.7 | 31 |
| HCC38 | 4.7 | 49 |

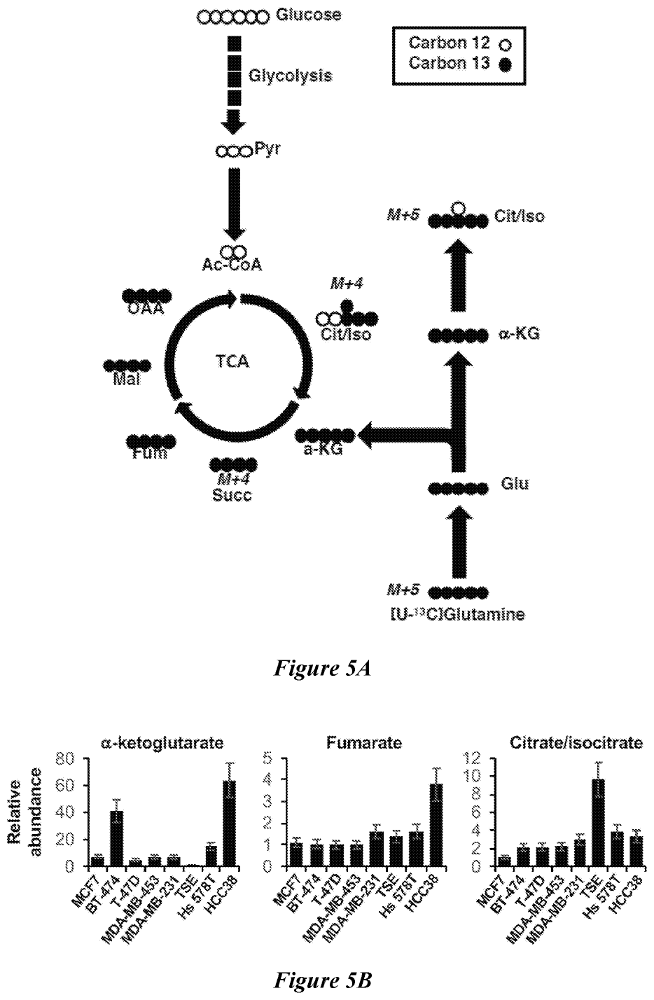

However, the sensitivity of breast cancer cells to GLS inhibitors does not correspond to their rate of glutamine consumption or to their expression of SLCIA5 (FIGS. 4B-4C), the major facilitator of glutamine uptake in breast cancer (Van Geldermalsen et al., “ASCT2/SLC1A5 Controls Glutamine Uptake and Tumour Growth in Triple-negative Basal-like Breast Cancer,” Oncogene 35:3201-3208 (2016), which is hereby incorporated by reference in its entirety). Therefore, stable isotope tracing was used to determine the fate of glutamine-derived carbon in the different breast cancer subtypes (FIG. 5A). Cells were cultured in medium containing uniformly labeled ¹³C-glutamine for 10 hours, and metabolites were then extracted for analysis by LC-HRMS. In both basal-subtype (MDA-MB-231 and TSE) and luminal-subtype (T-47D and MDA-MB-453) cells, glutamine is a major source of carbon for the TCA cycle (FIG. 4D). The proportion of the TCA cycle intermediates α-KG and fumarate derived directly from [U-³C]-glutamine (i.e. the abundance ratio of m+5 α-KG and m+4 fumarate) ranges from ˜30% to ˜55%. The abundance ratio of m+4 citrate ranges from 18 to 35%, whereas only trace quantities of m+5 citrate are present in all cell lines, indicating that the TCA cycle is turning in the oxidative direction under the aerobic experimental conditions. The abundance of TCA cycle metabolites showed no consistent differences between luminal- and basal-subtype cells (FIG. 5B), and samples collected at different time-points (1 hour, 24 hours) had similar labeling patterns to those collected at 10 hours (FIG. 5C). Thus, both luminal- and basal-subtype breast cancer cells make use of glutamine as an anaplerotic substrate, despite their contrasting sensitivity to GLS inhibitors.

### Example 2—Expression of GLS2 Is Elevated in Luminal-Subtype Breast Cancers

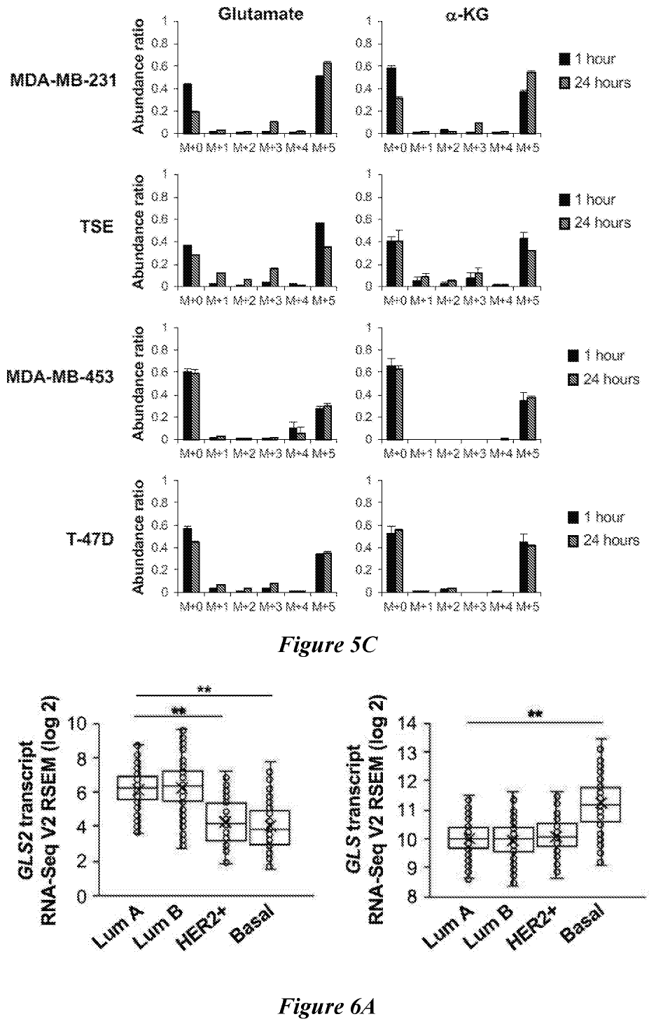

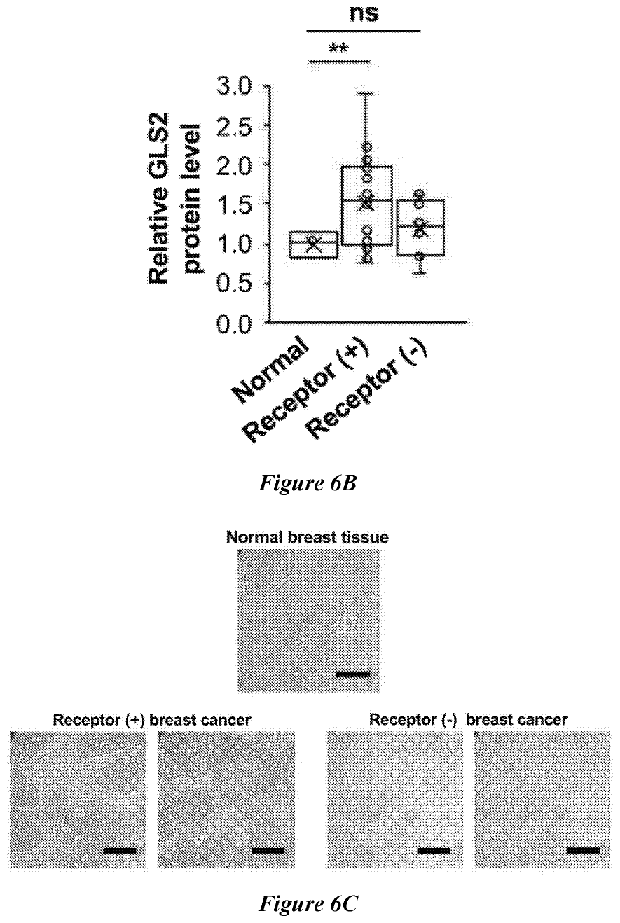

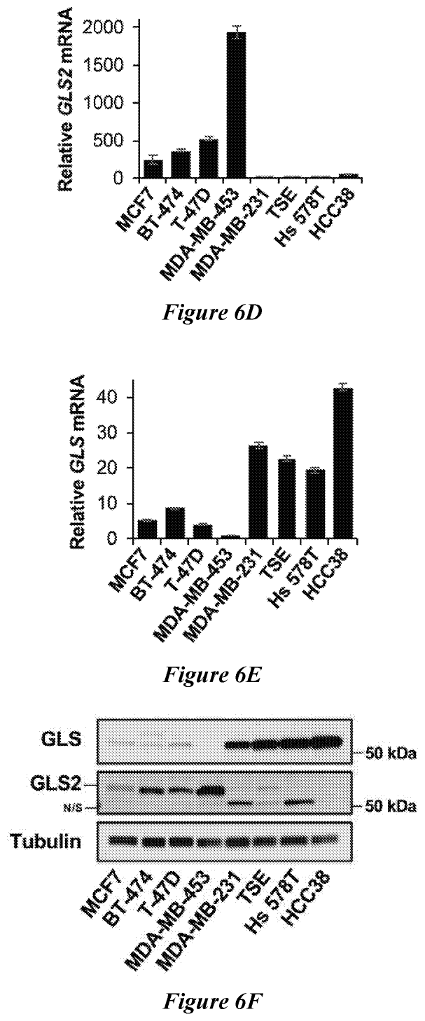

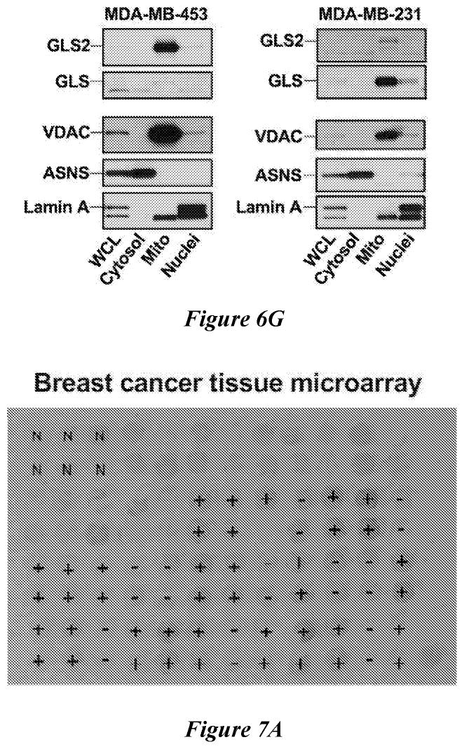

Previously, the glutaminase isozyme GLS2 has been described as a tumor suppressor in some contexts, with downregulated expression in liver and brain cancers (Matés et al., “Glutaminase Isoenzymes in the Metabolic Therapy of Cancer,” Biochim. Biophys. Acta—Rev. Cancer 1870:158-164 (2018), which is hereby incorporated by reference in its entirety). However, since luminal-subtype breast cancer cells resist GLS inhibitors yet still exhibit glutamine-mediated anaplerosis, it was hypothesized that they might instead be dependent on GLS2. The Cancer Genome Atlas (TCGA) invasive breast cancer dataset (Koboldt et al., “Comprehensive Molecular Portraits of Human Breast Tumours,” Nature 490:61-70 (2012), which is hereby incorporated by reference in its entirety) was used to examine GLS2 transcript levels in the breast cancer molecular subtypes luminal A (LumA), luminal B (LumB), HER2+, and basal. Expression of GLS2 is indeed substantially higher in LumA and LumB tumors than in basal-subtype tumors, which instead have high levels of the GLS transcript (FIG. 6A).

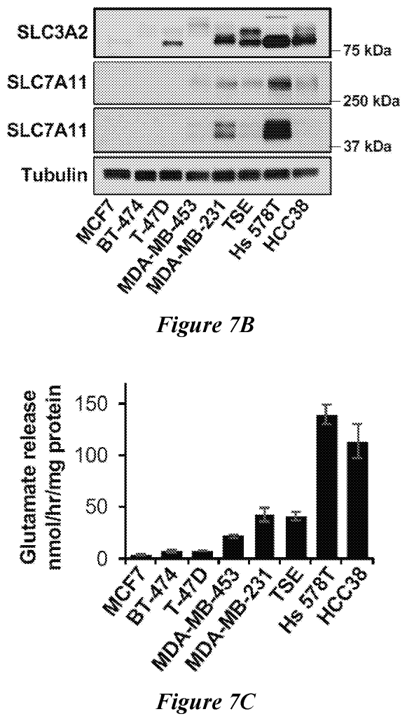

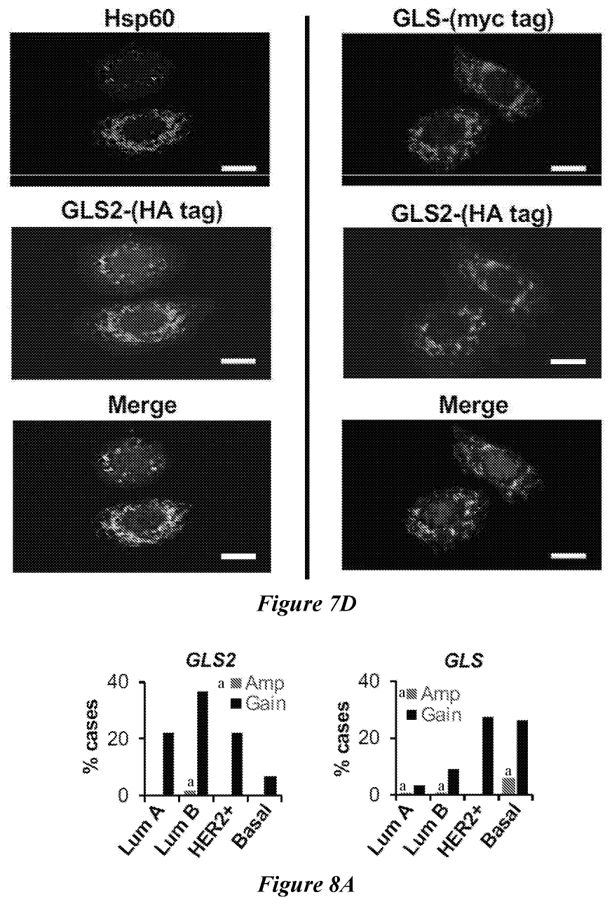

To compare protein levels of GLS2 in breast tumors and normal mammary tissue, a tissue microarray was probed (FIGS. 6B-6C and FIG. 7A). This revealed that GLS2 levels are significantly upregulated in receptor-positive breast tumors (the majority of which are luminal-subtype) relative to normal tissue but are elevated only in a small number of receptor-negative breast tumors (generally basal-subtype), with the mean level not differing significantly from normal tissue (FIG. 6B). Microscopic examination of the stained tissue slices revealed that in receptor-positive tumors, GLS2 is much more abundant in carcinoma cells than in neighboring connective tissue (FIG. 6C). It was then confirmed that the expression patterns for GLS and GLS2 are conserved in breast cancer cell lines. Quantitative real-time PCR (qRT-PCR) analysis showed that the GLS2 transcript is up to ˜2000-fold more abundant in luminal-subtype than in basal-subtype cells, which predominantly express GLS (FIGS. 6D-6E). These differences are conserved at the protein level (FIG. 6F). Consistent with other reports (Muir et al., “Environmental Cystine Drives Glutamine Anaplerosis and Sensitizes Cancer Cells to Glutaminase Inhibition,” Elife 6:e27713 (2017); Shin et al., “The Glutamate/Cystine xCT Antiporter Antagonizes Glutamine Metabolism and Reduces Nutrient Flexibility,” Nat. Commun. 8 (2017), which are hereby incorporated by reference in their entirety), high levels of GLS in basal-subtype cells are associated with expression of the glutamate/cystine antiport system ‘xCT’ and corresponding rapid glutamate efflux (FIGS. 7B-7C).

### Example 3—GLS2 Is Localized to Mitochondria in Breast Cancer Cells

Although GLS2 contains a predicted mitochondrial localization signal (Katt et al., “A Tale of Two Glutaminases: Homologous Enzymes with Distinct Roles in Tumorigenesis,” Future Med. Chem. 9:223-243 (2017), which is hereby incorporated by reference in its entirety), several subcellular localizations have been reported, including the nucleus in neurons and astrocytes and as a binding partner of the plasma membrane/cytosolic protein Rac1 in liver cancer cells (Cardona et al., “Expression of GIs and Gls2 Glutaminase Isoforms in Astrocytes,” Glia 63:365-382 (2015); Zhang et al., “Glutaminase 2 is a Novel Negative Regulator of Small GTPase Rac1 and Mediates p53 Function in Suppressing Metastasis,” Elife 5:e10727 (2016), which are hereby incorporated by reference in their entirety). To establish the localization of GLS2 in breast cancer cells, both MDA-MB-453 (high-GLS2) and MDA-MB-231 cells (high-GLS) were fractionated, and western blot analysis performed on the whole-cell lysates along with the cytosolic, mitochondrial, and nuclear fractions. As markers for these fractions, the samples were also probed for the cytosolic enzyme asparagine synthetase (ASNS), the mitochondrial ion channel VDAC, and the nuclear envelope protein lamin A. Both GLS2 and GLS, along with VDAC, were detected almost exclusively in the mitochondrial fractions of both cell lines (FIG. 6G). A small proportion of each glutaminase was present in the nuclear fractions, but the similar pattern for VDAC suggests that these signals arise from small quantities of mitochondria co-pelleting with nuclei (FIG. 6G). As expected, ASNS was detected in the cytosolic fraction, and lamin A in the nuclear fraction (FIG. 6G). The subcellular localization of ectopically-expressed GLS2 was also examined using immunofluorescence. Supporting a mitochondrial localization for GLS2 in breast cancer cells, GLS2-HA co-localizes with the endogenous mitochondrial marker protein Hsp60 in SK-BR-3 cells, as well as with ectopically-expressed GLS-myc (FIG. 7D).

### Example 4—GLS2 Expression Is Regulated by GATA3 and Promoter Methylation

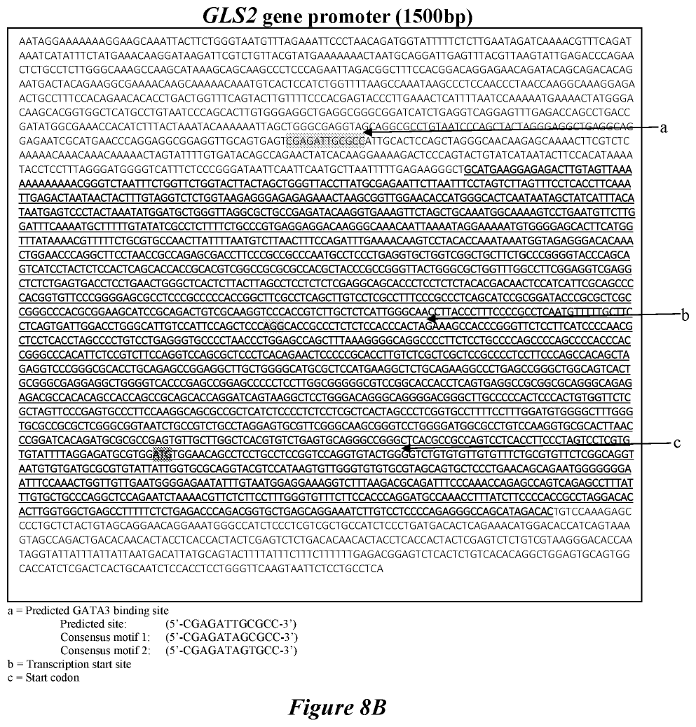

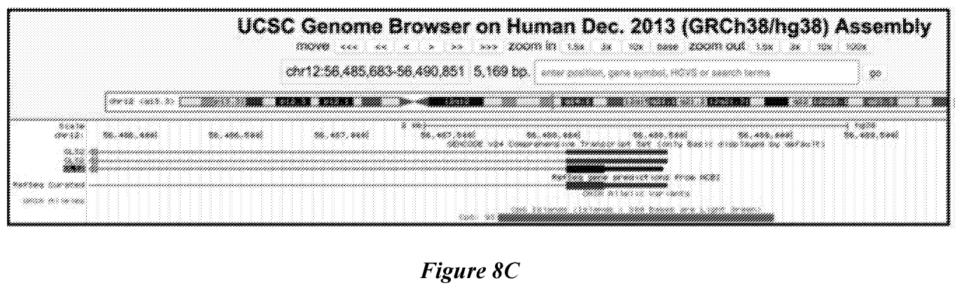

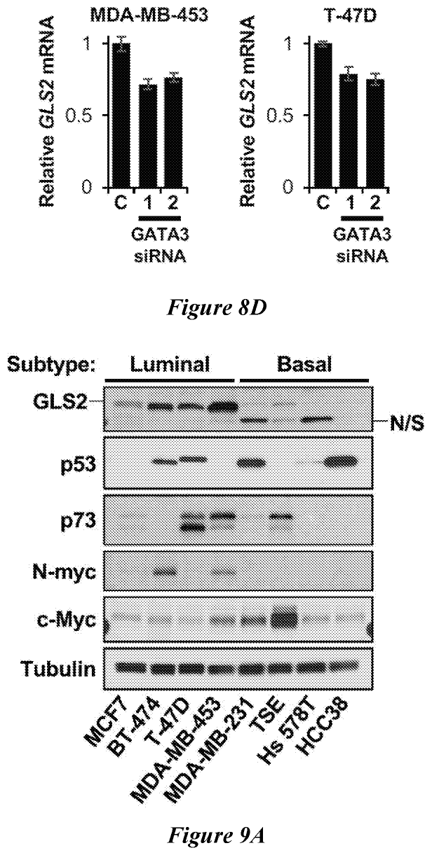

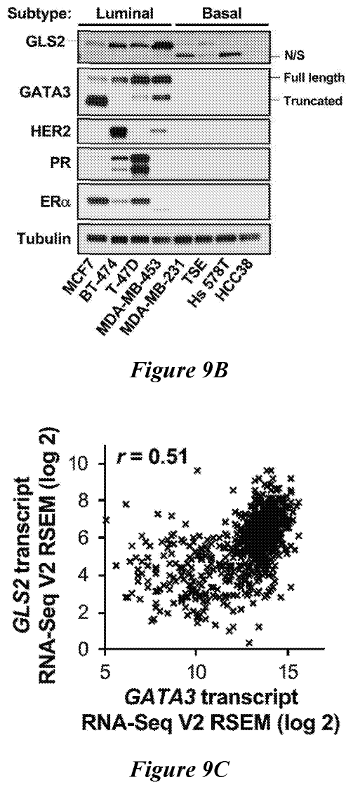

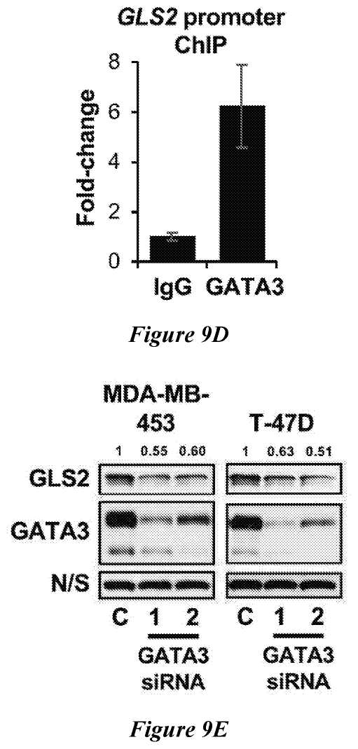

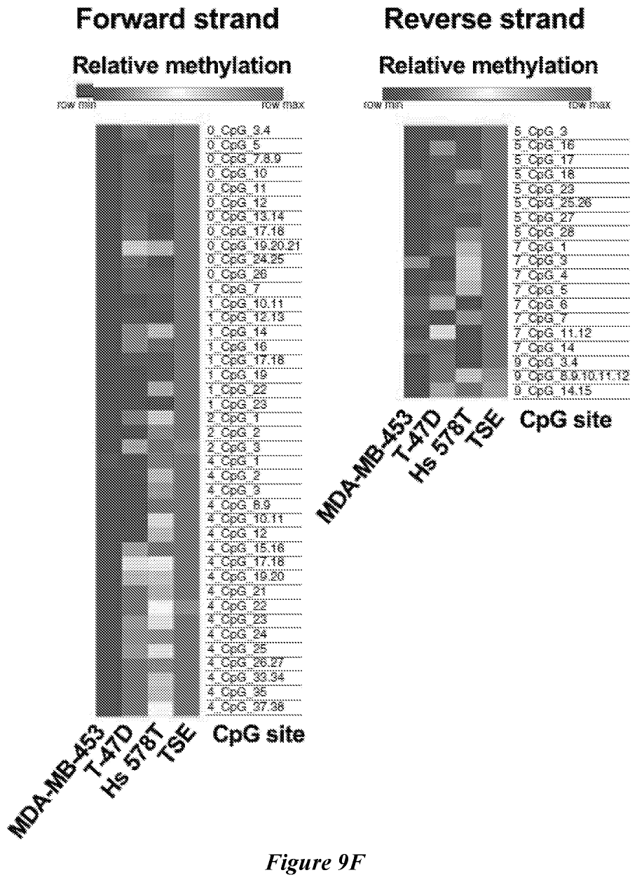

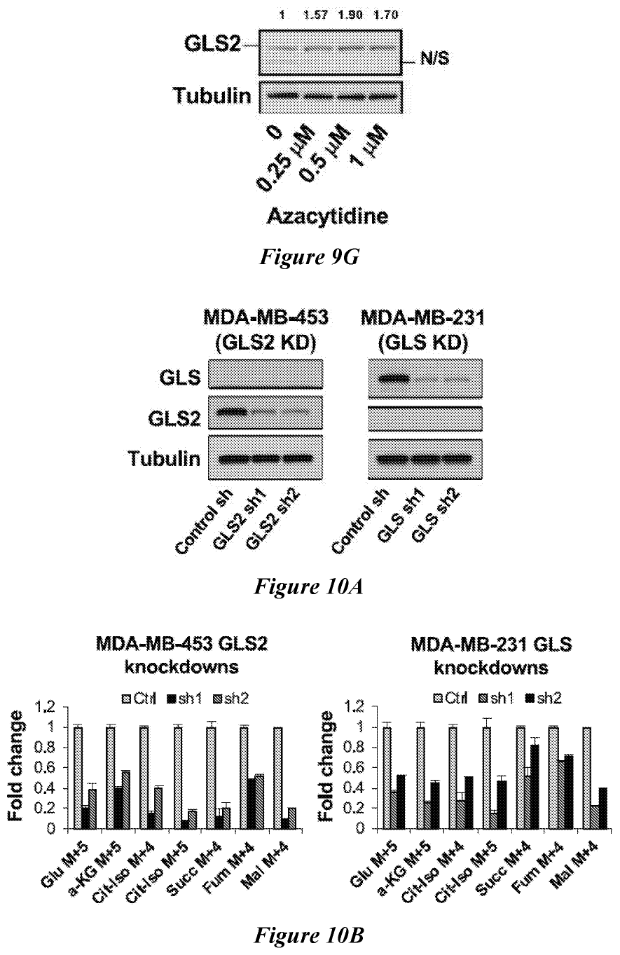

To understand the upregulation of GLS2 in luminal-subtype breast cancers, the mechanisms influencing GLS2 gene expression was investigated. Data from TCGA show a high frequency of copy-number gains at the GLS2 gene locus in luminal-subtype and HER2+ breast tumors (as high as 37% copy-number gain, 2% gene amplification, in the case of LumB), but not in basal-subtype tumors (FIG. 8A). The GLS locus exhibits the reverse pattern, with frequent copy-number gains in basal-subtype and HER2+ breast tumors, but not in LumA or LumB (FIG. 8A). The transcription factors p53, p63, p73, c-Myc, and N-myc have each been shown to regulate GLS2 expression in different contexts (Katt et al., “A Tale of Two Glutaminases: Homologous Enzymes with Distinct Roles in Tumorigenesis,” Future Med. Chem. 9:223-243 (2017), which is hereby incorporated by reference in its entirety). However, it was found that none of these regulators correlates with the pattern of GLS2 expression in breast cancer cells (FIG. 9A), and that the TP53 gene encoding p53 is mutated in BT-474, T-47D, MDA-MB-231, Hs 578T, and HCC38 cells. It was investigated whether additional factors contribute to the regulation of GLS2 expression in breast cancer.

Across the cell lines there is no clear association between GLS2 levels and any one of the receptors ER, PR, or HER2, with MDA-MB-453 cells, which have low expression of all three receptors, having the highest GLS2 levels (FIG. 9B). Since GLS2 is so abundant in LumA and LumB breast cancers relative to other subtypes, it was hypothesized that its expression is intrinsically associated with luminal cell status. Levels of GLS2 in breast cancer cell lines correlate closely with that of full-length GATA3 (FIG. 9B), a ‘master regulator’ of luminal differentiation (Asselin-Labat et al., “Gata-3 is an Essential Regulator of Mammary-Gland Morphogenesis and Luminal-cell Differentiation,” Nat. Cell Biol. 9:201-209 (2007), which is hereby incorporated by reference in its entirety). There is also a strong positive correlation between GATA3 and GLS2 transcript levels in human breast tumors (FIG. 9C). Therefore, PROMO (Farrd et al., “Identification of Patterns in Biological Sequences at the ALGGEN Server: PROMO and MALGEN.” Nucleic Acids Res. 31:3651-3653 (2003); Messeguer et al., “PROMO: Detection of Known Transcription Regulatory Elements using Species-Tailored Searches,” Bioinformatics 18:333-334 (2002), which are hereby incorporated by reference in their entirety) was used to analyze the human GLS2 gene promoter, and identified a match to the GATA3 consensus motif at position −1259 base pairs (bp) relative to the transcription start site (TSS) (FIG. 8B). The probability of this sequence occurring randomly within 1500 bp of the TSS is predicted by PROMO to be only 2.8%. Next a chromatin immunoprecipitation (ChIP) was carried out pulling down GATA3 from cross-linked chromatin prepared from MDA-MB-453 cell nuclei and using RT-PCR to quantify a 176-bp stretch of the GLS2 promoter centered on the GATA3 consensus motif. The GATA3 pulldown yielded a ˜6-fold higher signal relative to input than a negative-control IgG IP, consistent with direct binding of GATA3 to this region of the promoter (FIG. 9D). Finally, GATA3 was knocked down in MDA-MB-453 and T-47D cells and it was found that, after 48 hours, this resulted in a corresponding decrease in GLS2 levels in each cell line (FIGS. 9E and 8D). Thus, GATA3 contributes to the elevated expression of GLS2 in luminal-subtype breast cancers.

Because GLS2 levels are so low in basal-subtype breast cancers, it was also tested if expression is epigenetically silenced in these cells via methylation of the GLS2 gene promoter. After isolating genomic DNA from the basal-subtype cell lines with the lowest levels of GLS2 transcript (TSE and Hs 578T) and the luminal-subtype cell lines with the highest levels (T-47D and MDA-MB-453), the MassARRAY system was used to quantify methylation levels at sites in the CpG island centered on the TSS of the GLS2 gene (FIGS. 8B-8C). In MDA-MB-453 cells, which have the highest levels of GLS2, there is minimal promoter methylation, whereas in TSE cells, which have the lowest levels of GLS2 transcript, almost every site within the CpG island has a high methylation ratio (FIG. 9F). Treatment of TSE cells with the DNA hypomethylating agent azacitidine for 48 hours resulted in increased GLS2 protein levels (FIG. 9G). Collectively, these results show that GLS2 expression is regulated by GATA3 in luminal-subtype breast cancers, and potentially also by copy-number gains, whereas expression in basal-subtype breast cancers is repressed by promoter methylation.

### Example 5—GLS2 Mediates Glutamine Anaplerosis in Luminal-Subtype Cells

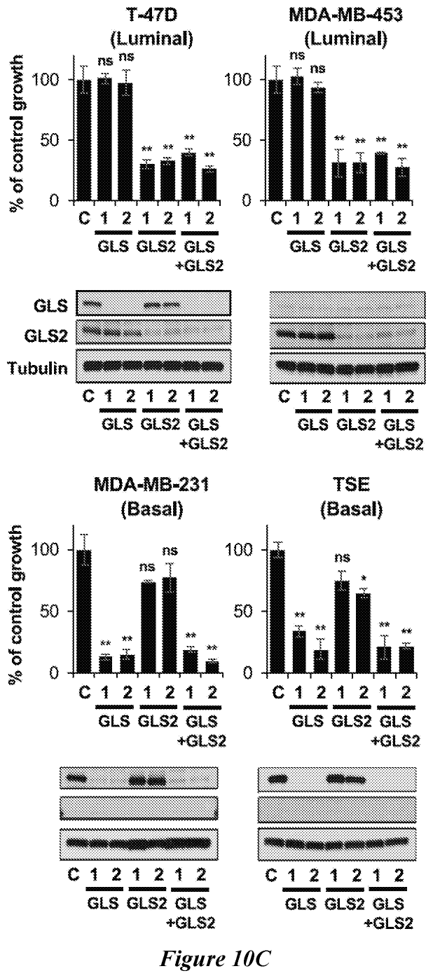

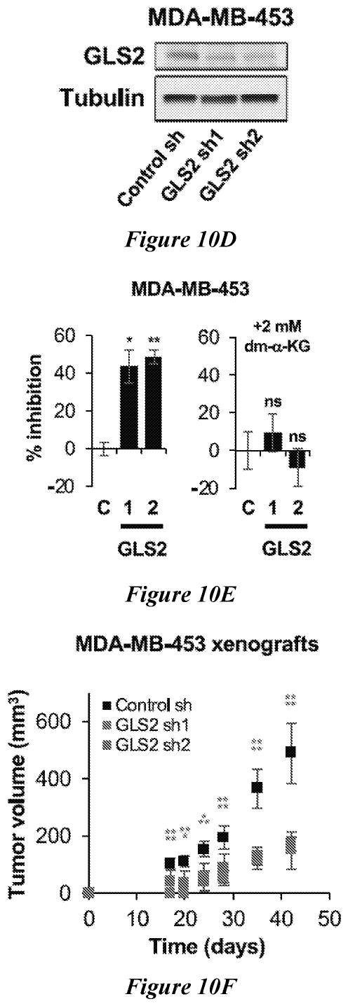

To probe the role of GLS2 in luminal-subtype cells, it was examined whether GLS2 is involved in the observed supply of glutamine-derived carbon to the TCA cycle (FIG. 4D). For comparison, the anaplerotic role of GLS in basal-subtype breast cancer cells was also quantified. MDA-MB-453 (luminal, high-GLS2) or MDA-MB-231 (basal, high-GLS) cells were transduced with constructs for expressing a control shRNA or shRNAs targeting GLS2 or GLS. After 48 hours, the knockdowns were confirmed using western blot analysis, and it was observed that within this time-frame there was no compensatory upregulation of the other glutaminase isozyme (FIG. 10A). Metabolomics experiments were then performed to assess the effects of the knockdowns on TCA cycle anaplerosis. Importantly, knockdown of GLS2 strongly inhibited glutamine-mediated TCA cycle anaplerosis in MDA-MB-453 cells (FIG. 10B). As expected, knockdown of GLS in MDA-MB-231 cells also suppressed the delivery of carbon from U-¹³C-glutamine into the TCA cycle (FIG. 10B). The glutaminase knockdowns also decreased the total abundance of glutamate and TCA cycle intermediates (FIG. 3A). As a control, the reciprocal experiment was carried out by treating MDA-MB-453 cells with GLS-targeted shRNAs, and MDA-MB-231 cells with GLS2-targeted shRNAs. In each case the abundance of glutamate and TCA cycle metabolites was minimally perturbed (FIG. 3B), further demonstrating that GLS2 is the predominant glutaminase in MDA-MB-453 cells and that GLS is the primary isozyme in MDA-MB-231 cells. Collectively, these findings establish that GLS2 is a metabolically active mitochondrial enzyme, critical for glutamine-mediated anaplerosis in luminal-subtype breast cancer cells.

### Example 6—GLS2 Is Essential in Luminal-Subtype Breast Cancer Cells

Because various functions have been reported for GLS2 in cancer, including tumor suppressive activity in liver cancer and glioblastoma (Mates et al., “Glutaminase Isoenzymes in the Metabolic Therapy of Cancer,” Biochim. Biophys. Acta—Rev. Cancer 1870:158-164 (2018), which is hereby incorporated by reference in its entirety), the importance of GLS2 for breast cancer cell proliferation and tumorigenesis was investigated. Luminal- and basal-subtype cell lines were transfected with either a control siRNA, or siRNAs selectively targeting GLS2, GLS, or both isozymes simultaneously. Western blot analysis 48 hours after transfection confirmed that potent and selective knockdowns had been achieved (FIG. 10C, lower panels). Cell proliferation was measured over 6 days. Knockdown of GLS2 strongly suppressed proliferation of luminal-subtype cells (MDA-MB-453 and T-47D), but only modestly impacted proliferation of basal-subtype cells (MDA-MB-231 and TSE) (FIG. 10C, upper panels). Knockdown of GLS had no effect on luminal-subtype cells but did inhibit basal-subtype cells, consistent with the results using GLS-selective inhibitors (FIG. 4A). In all cases the effects of GLS2 and/or GLS knockdowns could be rescued by supplementation of the culture medium with 2 mM dimethyl α-KG (dm-α-KG) (FIG. 3C), confirming that suppressed proliferation was a result of impaired TCA cycle anaplerosis.

Next, the importance of GLS2 for luminal-subtype breast tumor growth in vivo was addressed. This experiment required stable rather than transient depletion of GLS2, but potent shRNA-mediated knockdowns severely impacted luminal-subtype cell viability after several days. Therefore, a low titer of virus was used for transducing MDA-MB-453 cells and generated stable GLS2 partial-knockdown cell lines (FIG. 10D). Proliferation of the GLS2 partial-knockdown cells in culture was 40-50% slower than that of cells expressing a control shRNA (FIG. 10E, left panel), and could be rescued with 2 mM dm α-KG (FIG. 10E, right panel). The inventors then injected 3×10⁶ control or GLS2 partial-knockdown cells, in Matrigel suspension, into each flank of female NSG mice, and measured tumor growth over 42 days (n=6 tumors per condition). Both tumor initiation time and the rate of tumor growth were strongly inhibited in the GLS2 partial-knockdown samples relative to the control, providing a proof of principle that targeting GLS2 can inhibit luminal-subtype breast cancer tumorigenesis in vivo as well as cell proliferation ex vivo (FIG. 10F).

### Example 7—GLS2 Mediates Resistance to GLS Inhibitors

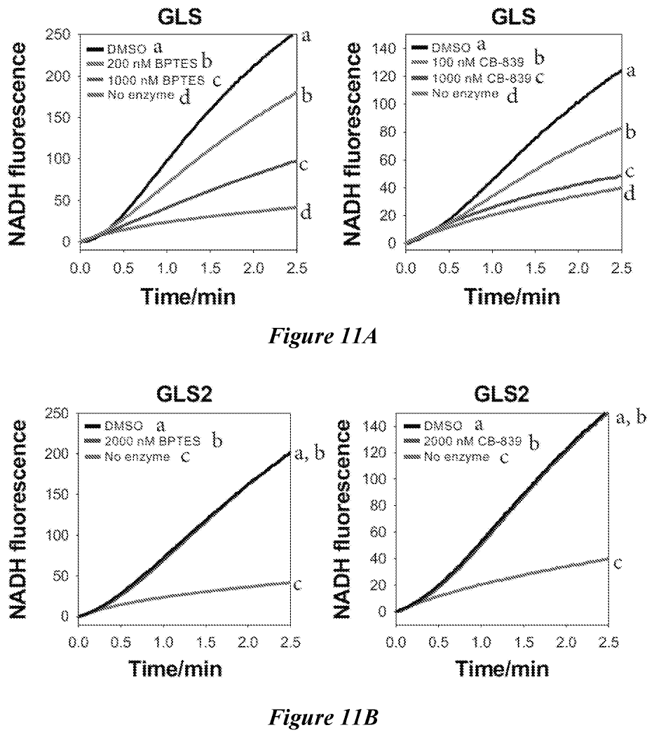

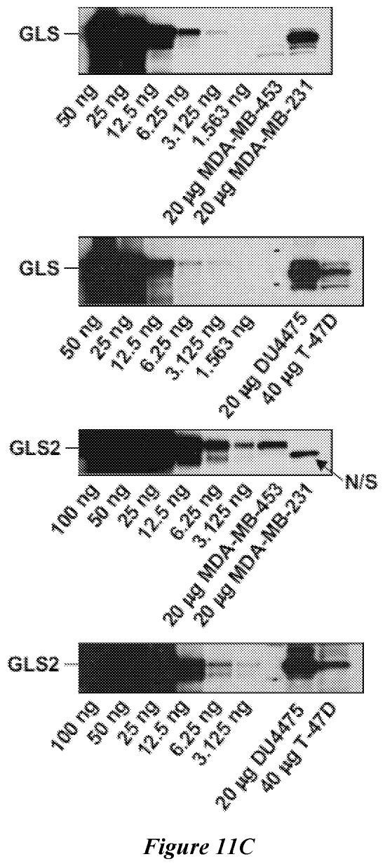

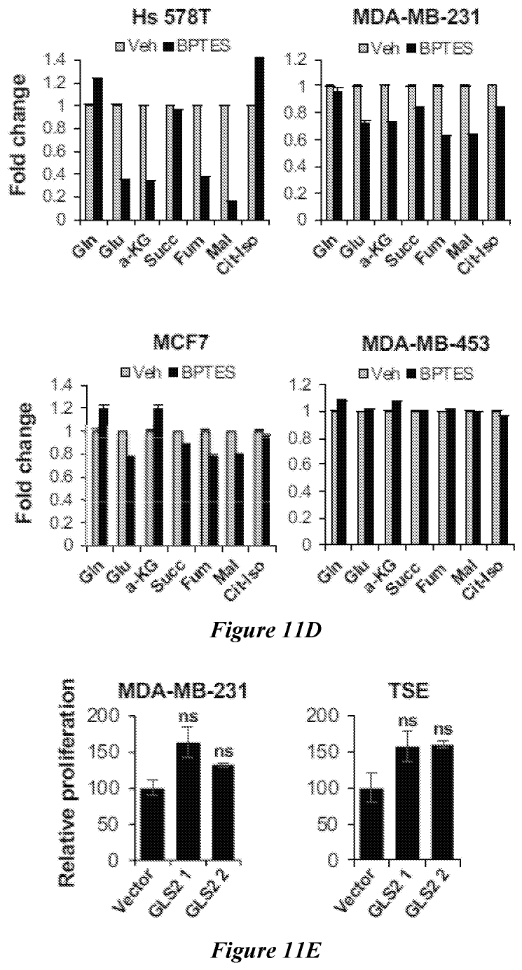

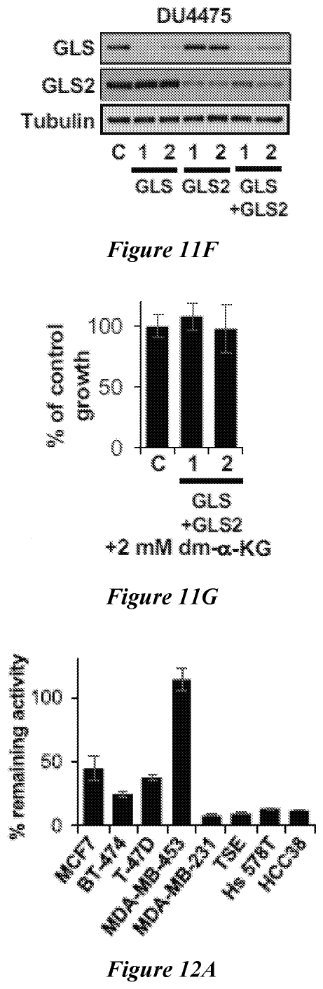

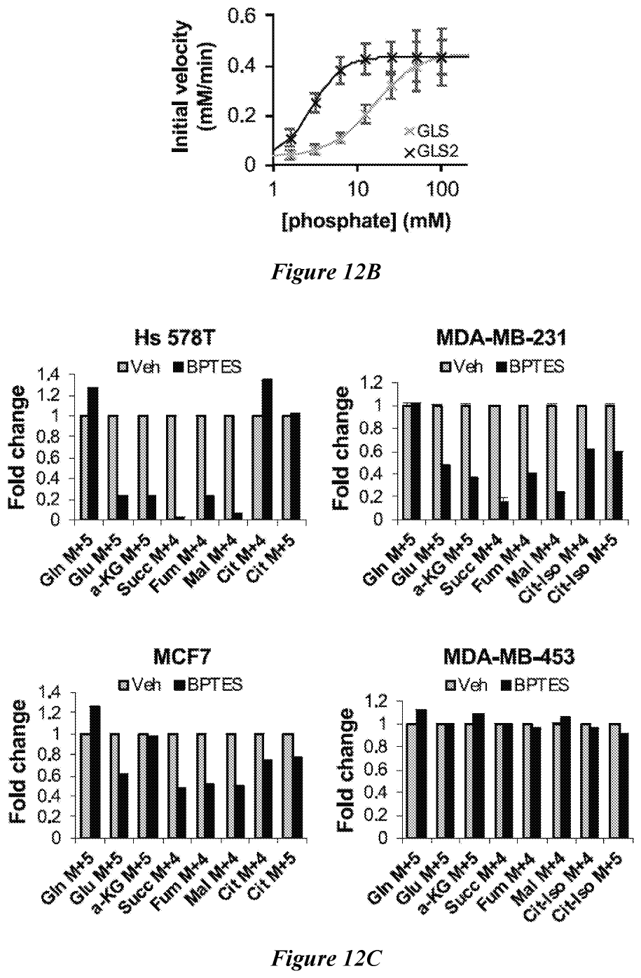

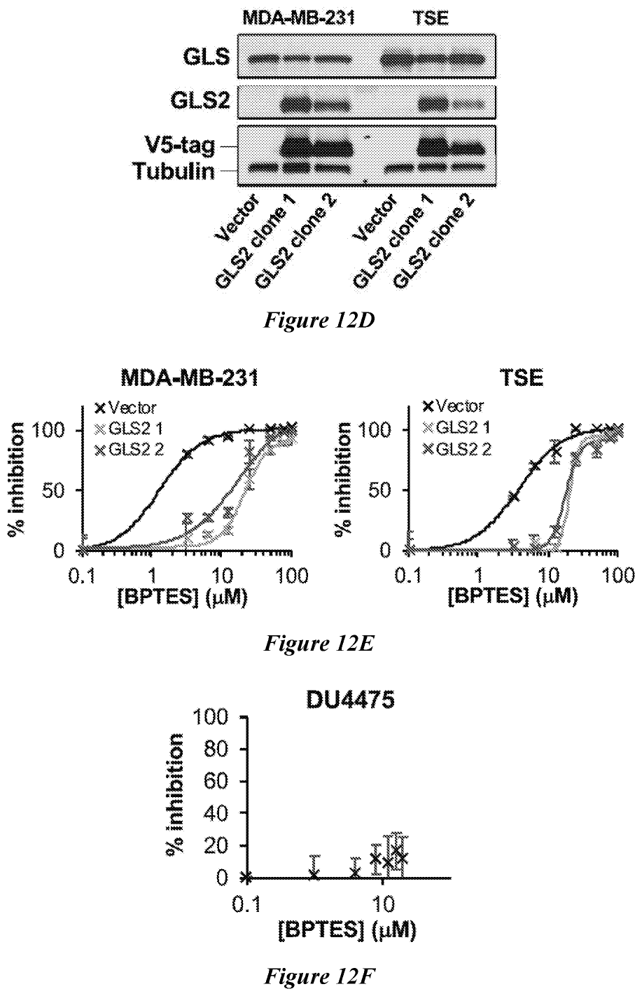

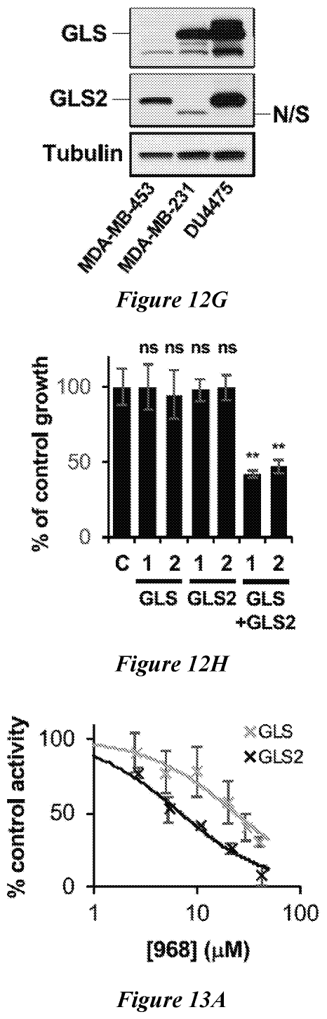

The BPTES class of inhibitors have been extensively studied and are highly selective for GLS over GLS2 (FIGS. 11A-11B) (Robinson et al., “Novel Mechanism of Inhibition of Rat Kidney-Type Glutaminase by Bis-2-(5-Phenylacetamido-1,2,4-Thiadiazol-2-yl)Ethyl Sulfide (BPTES),” Biochem. J. 406:407-414 (2007), which is hereby incorporated by reference in its entirety). Since GLS2 is capable of mediating TCA cycle anaplerosis in breast cancer cells, it was tested to determine if it is sufficient to confer resistance to these inhibitors. To start the inhibition of glutaminase activity in isolated mitochondria was studied. In mitochondria from luminal-subtype breast cancer cells, glutaminase activity was only partially inhibited by addition of 10 pM BPTES (FIG. 12A). In the case of MDA-MB-453 cells, BPTES treatment had no effect on activity. As expected, for mitochondria isolated from basal-subtype cells, glutaminase activity was almost completely abolished by 10 μM BPTES (FIG. 12A).

To gain further insight into the glutaminase expression profiles of breast cancer cells, western blot analyses were performed using known amounts of purified, recombinant GLS and GLS2 to estimate absolute levels of each glutaminase in cells (FIG. 11C). It was found that MDA-MB-453 cells contain ˜275 pg GLS2 per pg of total cellular protein and ˜75 pg/μg GLS. In contrast, MDA-MB-231 cells contain ˜500 pg/μg GLS and <75 pg/μg GLS2 (FIG. 11C). In T-47D cells, the absolute levels of GLS and GLS2 are approximately equal, at ˜200 pg/μg (FIG. 11C). However, GLS requires much higher concentrations of inorganic phosphate (Pi) to reach maximal activity (FIG. 12B), and in contrast to GLS2 it, is product inhibited by glutamate (Watford, M., “Hepatic Glutaminase Expression: Relationship to Kidney-Type Glutaminase and to the Urea Cycle,” FASEB J. 7:1468-1474 (1993), which is hereby incorporated by reference in its entirety). At physiological mitochondrial Pi concentrations of ˜10 mM (Hutson et al., “A ³¹P NMR Study of Mitochondrial Inorganic Phosphate Visibility: Effects of Ca²⁺, Mn²⁺, and the pH Gradient,” Biochemistry 31:1322-1330 (1992); Rauch et al., “Alteration of the Cytosolic-Mitochondrial Distribution of High-Energy Phosphates During Global Myocardial Ischemia May Contribute to Early Contractile Failure,” Circ. Res. 75:760-769 (1994), which are hereby incorporated by reference in their entirety), only GLS2 is fully activated (FIG. 12B).

Next cells were treated with 10 μM BPTES in culture medium containing [U-¹³C]-glutamine and extracted and analyzed cellular metabolites. Consistent with the data above, BPTES treatment potently inhibited the supply of glutamine-derived carbon to glutamate and the TCA cycle in the basal-subtype cell lines Hs 578T and MDA-MB-231 (FIG. 12C). In luminal-subtype MCF7 cells, which express GLS2 as well as GLS, BPTES treatment partially blocked glutamine anaplerosis (FIG. 12C). In MDA-MB-453 cells, which express primarily GLS2, BPTES treatment had no impact on glutamine-mediated anaplerosis (FIG. 12C). A similar pattern can be seen in the total abundances of TCA cycle intermediates (FIG. 11D).

Using MDA-MB-231 and TSE cells, which express almost exclusively GLS, derivative cell lines were generated that ectopically express GLS2 (FIG. 12D). In both cases, the clone with highest GLS2 expression showed a moderate decrease in the level of GLS. Although forced overexpression of GLS2 hinders the growth of liver cancer cells (Hu et al., “Glutaminase 2, a Novel p53 Target Gene Regulating Energy Metabolism and Antioxidant Function,” Proc. Nal. Acad. Sci. U.S.A. 107:7455-7460 (2010); Suzuki et al., “Phosphate-Activated Glutaminase (GLS2), a p53-Inducible Regulator of Glutamine Metabolism and Reactive Oxygen Species,” Proc. Natl. Acad. Sci. U.S.A. 107:7461-7466 (2010), which are hereby incorporated by reference in their entirety), in basal-subtype breast cancer cells each of the GLS2-overexpressing clones proliferated slightly more rapidly than control cells (FIG. 11E). Ectopic expression of GLS2 greatly decreased sensitivity to BPTES treatment, with the EC₅₀ shifting to >20 μM for both cell lines, which matches the aqueous solubility limit of the inhibitor (FIG. 12E and Table 8).

TABLE 8
EC50 values for BPTES and 968, derived
from 6-day cell proliferation assay.

| Cell line | BPTES EC₅₀/μM | 968 EC₅₀/μM |
|---|---|---|
| MDA-MB-231 (parental) | 2 | 4 |
| MDA-MB-231 vector only | 1.3 | 2 |
| MDA-MB-231 GLS2 clone 1 | >20 | 4 |
| MDA-MB-231 GLS2 clone 2 | >20 | 5 |
| TSE (parental) | 3 | 4 |
| TSE vector only | 4 | 2 |
| TSE GLS2 clone 1 | >20 | 7 |
| TSE GLS2 clone 2 | >20 | 6 |
| MCF7 | ~20 | 4 |
| BT-474 | ~20 | 4 |
| T-47D | ~20 | 4 |
| MDA-MB-453 | ~20 | 3 |
| MDA-MB-231 | 2.1 | 5 |
| TSE | 3.1 | 5 |
| Hs 578T | 3.7 | 4* |
| HCC38 | 4.7 | 4** |

*Maximal inhibition ~75%
**Maximal inhibition ~70%

Then a search was conducted for basal-subtype breast cancer cells that are intrinsically resistant to GLS-selective inhibitors, to test if resistance can be overcome by simultaneously targeting GLS2. The triple-negative, basal-subtype, breast cancer cell line DU4475, which was originally derived from a metastatic lesion, is highly resistant to BPTES treatment, with an EC₅₀ value >20 μM (FIG. 12F). Western blot analysis showed that DU4475 cells express higher levels of GLS than MDA-MB-231 cells, but simultaneously express higher levels of GLS2 than MDA-MB-453 cells (˜750 pg/μg of cellular protein for GLS, and ˜625 pg/μg for GLS2) (FIGS. 12G and 11C). Knockdown of either glutaminase isozyme alone did not impact DU4475 cell proliferation, but simultaneous knockdown of both GLS and GLS2 significantly inhibited growth (FIGS. 12H and 11F). Proliferation was again restored by supplementation of the culture medium with 2 mM dm-α-KG (FIG. 11G).

### Example 8-968 Inhibits GLS2 and Suppresses BPTES-Resistant Breast Cancer Growth

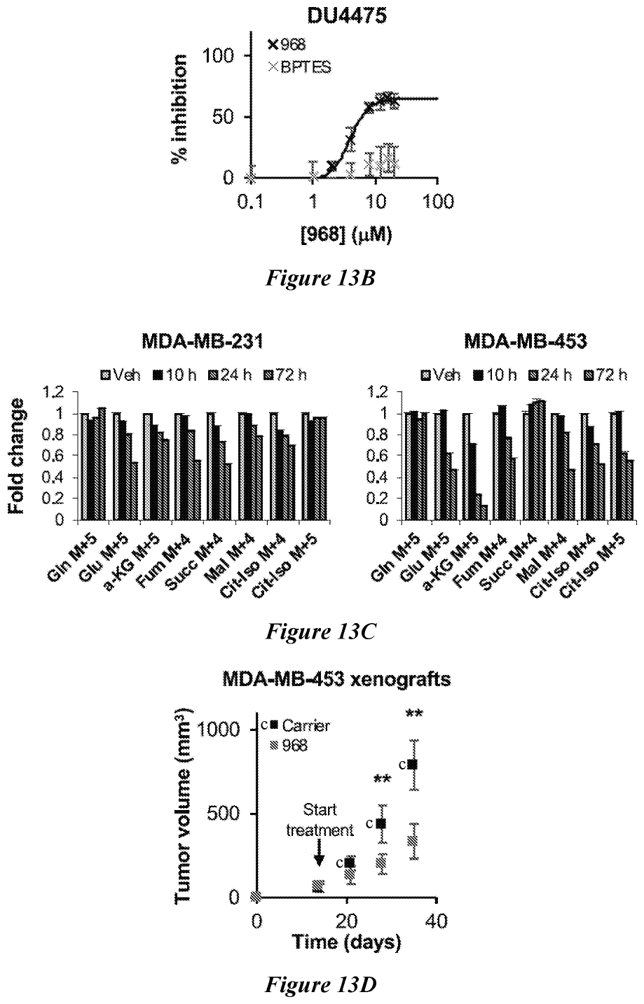

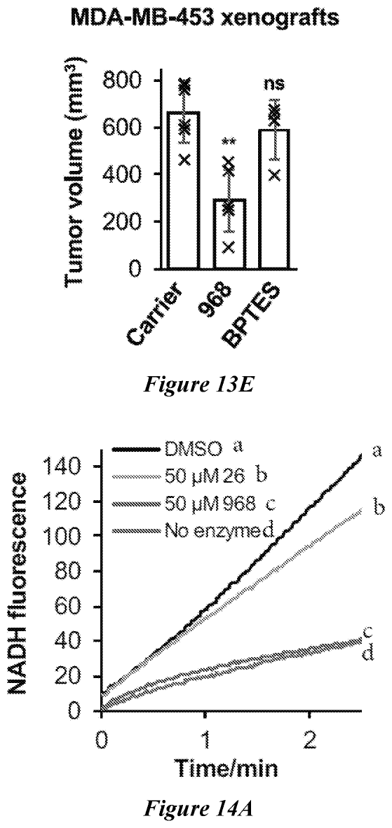

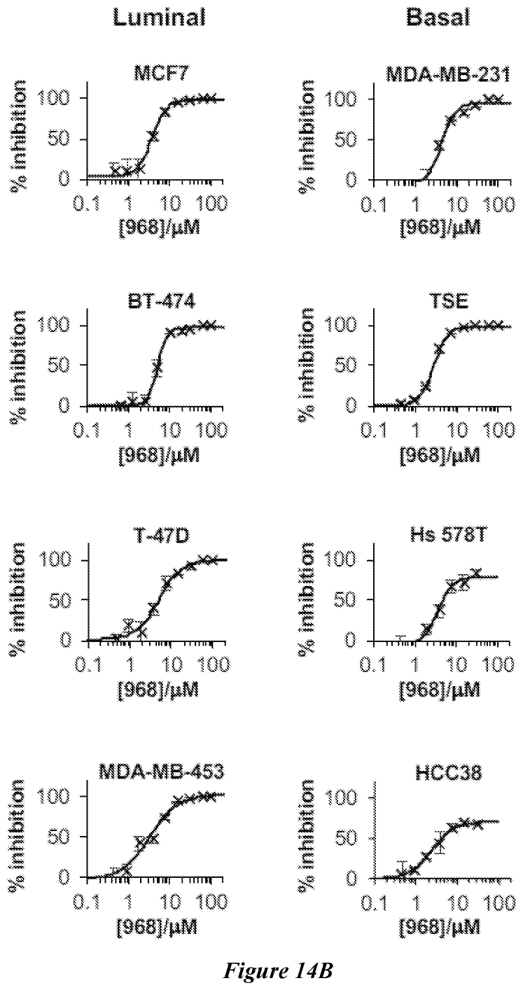

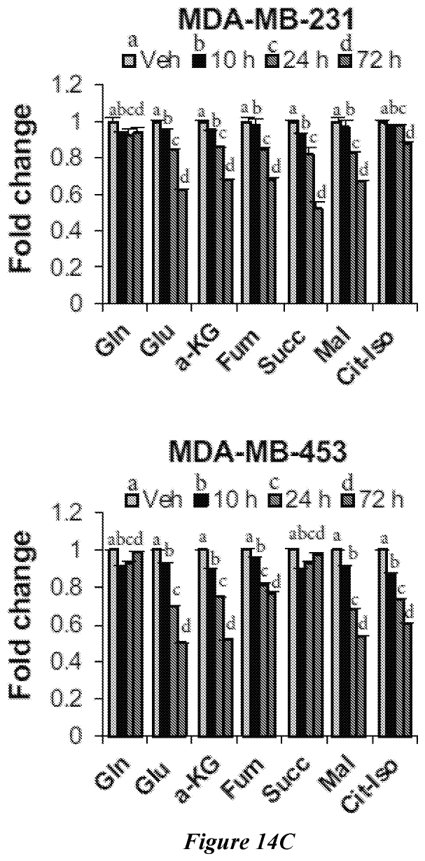

Previously, it was reported that the small molecule 968 binds and inhibits the GLS splice variant GAC (Wang et al., “Targeting Mitochondrial Glutaminase Activity Inhibits Oncogenic Transformation,” Cancer Cell 18:207-219 (2010), which is hereby incorporated by reference in its entirety). This inhibitor has a much higher affinity for monomeric GAC than for the active tetramer, and is proposed to bind to newly-synthesized enzyme monomers and prevent the formation of activated tetramers (Stalnecker et al., “Mechanism by which a Recently Discovered Allosteric Inhibitor Blocks Glutamine Metabolism in Transformed Cells,” Proc. Natl. Acad. Sci. U.S.A. 112:394-399 (2014), which is hereby incorporated by reference in its entirety). However, the sensitivity of GLS2 to 968 has not previously been tested. Therefore, the effect of 968 on the activity of purified, recombinant, glutaminase was measured. In contrast to BPTES-class inhibitors, which are highly selective for GLS, 968 inhibited both GLS and GLS2, with a moderate (>3-fold) selectivity for GLS2 (FIG. 13A). Compound 26, a quinoline derivative of 968 which differs by only a single carbon-to-nitrogen substitution, has greatly weakened affinity for GLS (Katt et al., “Dibenzophenanthridines as Inhibitors of Glutaminase C and Cancer Cell Proliferation,” Mol. Cancer Ther. 11:1269-1278 (2012); Stalnecker et al., “Mechanism by which a Recently Discovered Allosteric Inhibitor Blocks Glutamine Metabolism in Transformed Cells,” Proc. Natl. Acad. Sci. U.S.A. 112:394-399 (2014), which are hereby incorporated by reference in their entirety). Similarly, it was found that compound 26 minimally inhibits recombinant GLS2 at 50 μM, whereas 968 completely blocks activity at this concentration (FIG. 14A).

Treatment of breast cancer cells with 968 inhibited proliferation with EC₅₀ values of 3-5 μM (FIG. 14B and Table 8). For the cells with highest expression of GLS, maximal inhibition was ˜70-75%. The inactive analog compound 26 did not inhibit the proliferation of any of the cell lines it was tested it against. Despite their intrinsic resistance to BPTES, DU4475 cells responded to 968 treatment with an EC₅₀ value of 4 μM (FIG. 13B). The response of the GLS2-overexpressing derivatives of MDA-MB-231 and TSE cells was also measured, described above. Consistent with the ability of 968 to inhibit both glutaminase isozymes, overexpression of GLS2 had little effect on the EC₅₀ values for 968 (Table 8).

Next basal- and luminal-subtype breast cancer cells were treated with 10 μM 968 and extracted metabolites for analysis at different time points. Consistent with the mechanism of 968 binding to monomeric glutaminase and preventing the formation of active tetramers, a time-dependent inhibition of glutamine-mediated TCA cycle anaplerosis was observed (FIG. 13C). Matching the partial selectivity of 968 for GLS2 over GLS, the effects were more pronounced in MDA-MB-453 cells than MDA-MB-231 cells (FIG. 13C). Treatment with 968 also decreased the total abundance of TCA cycle intermediates (FIG. 14C).

Since 968 inhibits the proliferation of breast cancer cells ex vivo but does not affect the growth of primary human mammary epithelial cells or fibroblasts (Wang et al., “Targeting Mitochondrial Glutaminase Activity Inhibits Oncogenic Transformation,” Cancer Cell 18:207-219 (2010), which is hereby incorporated by reference in its entirety), it was tested if it can be used to treat luminal-subtype breast tumor growth in vivo. 3×10⁶ MDA-MB-453 cells in Matrigel suspension was injected into each flank of NSG mice (n=6 xenografts per condition) and waited until palpable tumors 1-2 mm in diameter were present (14 days). At this point, mice were separated into two groups, with one group receiving subcutaneous injections of 968 (10 mg/kg body weight) 3 times per week, and the other group receiving carrier solution only. The tumor growth was monitored for three weeks after initiating treatment and found that it was robustly inhibited in 968-treated animals (FIG. 13D). In contrast, treatment of animals with 10 mg/kg BPTES did not significantly impact tumor growth (FIG. 13E). Thus, 968 inhibits GLS2 in vitro, suppresses GLS2-mediated anaplerosis, and is effective against BPTES-resistant breast cancer cell proliferation and tumorigenesis. Taken together, the results reveal an essential and potentially druggable role for GLS2 in luminal-subtype breast cancers.

### Discussion of Examples 1-8

Efforts to target glutamine catabolism for cancer therapy have focused on inhibiting the glutaminase isozyme GLS, which is highly expressed and onco-supportive in diverse malignancies (Cluntun et al., “Glutamine Metabolism in Cancer: Understanding the Heterogeneity,” Trends in Cancer 3:169-180 (2017), which is hereby incorporated by reference in its entirety). The importance of the other mammalian glutaminase, GLS2, in tumorigenesis has remained less clear, and various subcellular localizations and functions have been described, including tumor suppressor activity (Mates et al., “Glutaminase Isoenzymes in the Metabolic Therapy of Cancer,” Biochim. Biophys. Acta—Rev. Cancer 1870:158-164 (2018), which is hereby incorporated by reference in its entirety). It is reported herein that GLS2 is upregulated in luminal-subtype/receptor-positive breast cancers, where it is essential for glutamine-mediated TCA cycle anaplerosis, cell proliferation, and tumorigenesis. These findings explain the identification of GLS2 as one of only 16 metabolic enzymes required for tumorigenesis in an earlier functional genomics screen (Possemato et al., “Functional Genomics Reveal that the Serine Synthesis Pathway is Essential in Breast Cancer,” Nature 476:346-350 (2011), which is hereby incorporated by reference in its entirety).

Previous studies found that triple-negative, but not receptor-positive, breast cancer cell lines often express high levels of the GLS splice variant GAC (Gross et al., “Antitumor Activity of the Glutaminase Inhibitor CB-839 in Triple-negative Breast Cancer,” Mol. Cancer Ther. 13:890-901 (2014), which is hereby incorporated by reference in its entirety). The results herein indicate that, rather than being directly dictated by the cellular receptor status, differences in glutaminase expression in breast cancer correspond to the intrinsic molecular subtype (FIG. 6). Specifically, LumA and LumB breast tumors display relatively high levels of GLS2, whereas GLS is elevated in basal-subtype breast tumors. Since expression of the GLS2 gene is driven in part by the transcription factor GATA3 (see FIGS. 9A-9G), a master-regulator of luminal differentiation, high levels of GLS2 might be intrinsically associated with luminal cell status. Expression of both glutaminase isozymes is low in HER2+ breast tumors, suggesting that glutaminase-mediated TCA cycle anaplerosis might not be a major metabolic pathway in this disease subtype.

These findings highlight the importance of considering GLS2 when identifying target diseases for treatment with GLS-selective inhibitors. Gene expression data from TCGA show that GLS2 transcript levels are consistently upregulated relative to healthy tissue in colorectal tumors and also in a subset of lung tumors. With clinical trials underway to evaluate the efficacy of CB-839 against these cancers, it will be important to characterize further the function of GLS2 in these contexts. In pancreatic ductal adenocarcinoma (PDAC) cells, GLS inhibitors have only a temporary cytostatic effect which is followed by metabolic adaptation and recovery of proliferation (Biancur et al., “Compensatory Metabolic Networks in Pancreatic Cancers Upon Perturbation of Glutamine Metabolism,” Nat. Commun. 8:15965 (2017), which is hereby incorporated by reference in its entirety). Notably, GLS2 is present in both healthy pancreas and PDAC cells (Altman et al., “From Krebs to Clinic: Glutamine Metabolism to Cancer Therapy,” Nat. Rev. Cancer 16:619-634 (2016); Biancur et al., “Compensatory Metabolic Networks in Pancreatic Cancers Upon Perturbation of Glutamine Metabolism,” Nat. Commun. 8:15965 (2017), which are hereby incorporated by reference in their entirety), and thus might provide a critical supply of glutamate following GLS inhibition. The sensitivity of some cancer cells to GLS inhibitors requires high expression of the xCT antiporter, which exchanges intracellular glutamate for extracellular cystine and can therefore deplete intracellular glutamate reserves (Muir et al., “Environmental Cystine Drives Glutamine Anaplerosis and Sensitizes Cancer Cells to Glutaminase Inhibition,” Elife 6:e27713 (2017), which is hereby incorporated by reference in its entirety). Thus, a signature of concurrent high expression of GLS and xCT, along with low levels of GLS2, might identify tumors that are most likely to respond to GLS-targeted therapy.

### GLS2 as a Potential Therapeutic Target

In healthy tissues GLS2 expression is highest in periportal regions of the liver, where it allows glutamine carbon to be directed via the TCA cycle into the gluconeogenic pathway in response to glucagon (Lacey et al., “Increased Activity of Phosphate-Dependent Glutaminase in Liver Mitochondria as a Result of Glucagon Treatment of Rats,” Biochem. J. 194:29-33 (1981); Watford and Smith, “Distribution of Hepatic Glutaminase Activity and mRNA in Perivenous and Periportal Rat Hepatocytes,” Biochem. J. 267:265-267 (1990), which are hereby incorporated by reference in their entirety). For GLS2 to be targeted for cancer therapy, any toxicity arising from inhibiting its normal physiological function must be within a tolerable range. It was observed that mice treated with 968 at 10 mg/kg body weight, three times weekly for three weeks, showed no gross evidence of toxicity. Moreover, it was recently reported that GLS2 knockout mice are viable, albeit with a decreased ability to maintain plasma glucose levels during fasting (Miller et al., “Targeting Hepatic Glutaminase Activity to Ameliorate Hyperglycemia,” Nat. Med. 24:518-524 (2018), which is hereby incorporated by reference in its entirety). These results indicate that GLS2 could be safely targeted as a strategy for treating luminal-subtype breast cancers, most likely as part of a combination therapy designed to maximize cancer cell dependence on the glutaminase reaction. To date, drug discovery efforts for blocking glutamine catabolism in cancer have focused almost exclusively on the GLS isozyme. However, a small number of molecular scaffolds have now been reported to inhibit GLS2 (Wu et al., “Glutaminase Inhibitors: A Patent Review,” Expert Opin. Ther. Pat. 28:823-835 (2018), which is hereby incorporated by reference in its entirety), including 968, which is shown to target both isozymes with a moderate (˜3-fold) selectivity for GLS2. The finding that GLS2 is upregulated and essential in the most prevalent subtypes of breast cancer support the notion of building on these scaffolds to develop more potent inhibitors for selective targeting of GLS2-high cancers.

Humans have two genes encoding glutaminase enzymes, GIS and GLS2. Efforts to target glutamine catabolism for cancer therapy have focused on GLS, an inhibitor of which (CB-839) is currently in clinical trials. The GLS2 isozyme has previously been described as a tumor suppressor, with downregulated expression in liver cancer. It is reported herein that GLS2 is overexpressed and essential for growth in the most prevalent subtypes of breast cancer, luminal A and B. Although GLS2 is insensitive to CB-839-class drugs, it is inhibited by the small molecule 968, which suppresses breast tumor growth in vivo. These findings establish a critical role for GLS2 in breast cancer and advance the understanding of how to target aberrant glutamine metabolism for cancer therapy generally.

Preferences and options for a given aspect, feature, embodiment, or parameter of the methods described herein should, unless the context indicates otherwise, be regarded as having been disclosed in combination with any and all preferences and options for all other aspects, features, embodiments, and parameters of the methods described herein.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

1. A method of reducing the production of glutamate from glutamine by GLS and by GLS2 in a cancerous cell or cancerous tissue, said method comprising:
inhibiting glutaminase activity of GLS and GLS2 in the cancerous cell or cancerous tissue by a method comprising:
selecting a cancerous cell or cancerous tissue; and
contacting GLS and GLS2 in the cell or tissue with a dual GLS/GLS2 inhibitor;
wherein said contacting reduces the production of glutamate from glutamine by GLS and by GLS2 in the cell or tissue.

2. A method of treating a subject with a condition mediated by production of glutamate from glutamine by GLS and by GLS2, said method comprising:
selecting a subject with a condition mediated by production of glutamate from glutamine by GLS and by GLS2 and
administering to said selected subject a dual GLS/GLS2 inhibitor under conditions effective to treat the condition mediated by production of glutamate from glutamine.

3. A method of reducing the production of glutamate from glutamine by GLS2 in a cancerous cell or cancerous tissue, said method comprising:
inhibiting glutaminase activity of GLS2 in the cancerous cell or cancerous tissue by a method comprising:
selecting a cancerous cell or cancerous tissue characterized by GLS2 overexpression and/or GLS2 hyperactivity; and
contacting GLS2 in the cell or tissue with an inhibitor of GLS2 glutaminase activity;
wherein said contacting reduces the production of glutamate from glutamine by GLS2 in the cell or tissue.

4. A method of treating a subject with a condition mediated by production of glutamate from glutamine by GLS2, said method comprising:
selecting a subject with a condition mediated by production of glutamate from glutamine by GLS2 and
administering to said selected subject an inhibitor of GLS2 glutaminase activity under conditions effective to treat the condition mediated by production of glutamate from glutamine.

5. The method according to claim 3, wherein the inhibitor is a dual GLS/GLS2 inhibitor.

6. The method according to claim 1, wherein the inhibitor inhibits GLS2 and GAC, and/or inhibits GLS2 and KGA, and/or inhibits LGA, and/or inhibits GAB.

7-9. (canceled)

10. The method according to claim 1, wherein the inhibitor is a compound, or a pharmaceutically acceptable salt, ester, enol ether, enol ester, solvate, hydrate, or prodrug thereof, selected from the group consisting of:
I) compounds of Formula IA: 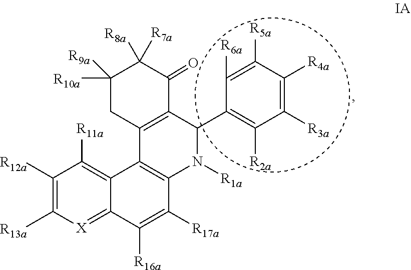
wherein:
the dotted circle identifies an active moiety;
X is independently —CR₁₄ₐ— or N;
R₁ₐ is independently H, —OH, —OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₂ₐ, R₃ₐ, R₄ₐ, R₅ₐ, and R₆ₐ are each independently a photoreactive moiety, H, halogen, —NO₂, —OH, —OR₁₄ₐ, —SR₁₄ₐ, —NH₂, —NHR₁₄ₐ, —NR₁₄ₐR₁₅ₐ, R₁₄ₐC(O)—, R₁₄ₐOC(O)—, R₁₄ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; or R₂ₐ and R₃ₐ, R₃ₐ and R₄ₐ, R₄ₐ and R₅ₐ, or R₅ₐ and R₆ₐ are combined to form a heterocyclic ring optionally substituted with a photoreactive moiety;
R₇ₐ, R₈ₐ, R₉ₐ, and R₁₀ₐ are each independently a photoreactive moiety, H, —OH, —NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl are optionally substituted from 1 to 3 times with substituents selected from the group consisting of, halogen, —OH, —NH₂, C₁-C₆alkyl, C₂-C₆ alkenyl, C₁-C₆ alkoxy, —SH, and C₁-C₆ thioalkyl, and wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; and
R₁₁ₐ, R₁₂ₐ, R₁₃ₐ, R₁₄ₐ, R₁₅ₐ, R₁₆ₐ, and R₁₇ₐ are each independently a photoreactive moiety, H, halogen, —OH, —O—C₁-C₆ alkyl, —O—C₂-C₆ alkenyl, —O—C₂-C₆ alkynyl, —NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, wherein the alkyl, alkenyl, alkynyl, cycloalkyl, cycloalkylalkyl, arylalkyl, and mono or polycyclic aryl are optionally substituted with a photoreactive moiety and each one of R₁₁ₐ-R₁₇ₐ is optionally substituted with —NH₂, —OH, halogen, —COOH, —NO₂, or —CN;
II) compounds of Formula IB: 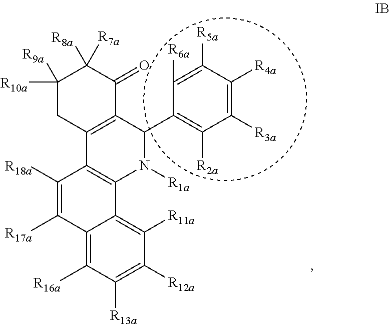
wherein:
the dotted circle identifies an active moiety;
R₁ₐ is H, —OH, —OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₂ₐ, R₃ₐ, R₄ₐ, R₅ₐ, and R₆ₐ are each independently a photoreactive moiety, H, halogen, —NO₂, —OH, —OR₁₄ₐ, —SR₁₄ₐ, —NH₂, —NHR₁₄ₐ, —NR₁₄ₐR₁₅ₐ, R₁₄ₐC(O)—, R₁₄ₐOC(O)—, R₁₄ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; or R₂ₐ, and R₃ₐ, R₃ₐ and R₄ₐ, R₄ₐ and R₅ₐ, or R₅ₐ and R₆ₐ are combined to form a heterocyclic ring optionally substituted with a photoreactive moiety; wherein at least two of R₂ₐ, R₃ₐ, R₄ₐ, R₅ₐ, and R₆ₐ are not hydrogen;
R₇ₐ, R₈ₐ, R₉ₐ, and R₁₀ₐ are each independently a photoreactive moiety, H, —OH, —NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl are optionally substituted from 1 to 3 times with substituents selected from the group consisting of, halogen, —OH, —NH₂, C₁-C₆alkyl, C₂-C₆ alkenyl, C₁-C₆ alkoxy, —SH, and C₁-C₆ thioalkyl, and wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; and
R₁₁ₐ, R₁₂ₐ, R₁₃ₐ, R₁₄ₐ, R₁₅ₐ, R₁₆ₐ, R₁₇ₐ, and R₁₈ₐ are each independently a photoreactive moiety, H, halogen, —OH, —O—C₁-C₆ alkyl, —O—C₂-C₆ alkenyl, —O—C₂-C₆ alkynyl, —NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, wherein the alkyl, alkenyl, alkynyl, cycloalkyl, cycloalkylalkyl, arylalkyl, and mono or polycyclic aryl are optionally substituted with a photoreactive moiety and each one of R₁₁ₐ-R₁₈ₐ is optionally substituted with —NH₂, —OH, halogen, —COOH, —NO₂, or —CN;
III) compounds of Formula IC: 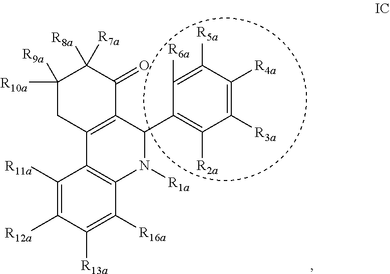
wherein:
the dotted circle identifies an active moiety;
R₁ₐ is H, —OH, —OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₂ₐ, R₃ₐ, R₄ₐ, R₅ₐ, and R₆ₐ are each independently a photoreactive moiety, H, halogen, —NO₂, —OH, —OR₁₄ₐ, —SR₁₄ₐ, —NH₂, —NHR₁₄ₐ, —NR₁₄ₐR₁₅ₐ, R₁₄ₐC(O)—, R₁₄ₐOC(O)—, R₁₄ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; or R₂ₐ and R₃ₐ, R₃ₐ and R₄ₐ, R₄ₐ and R₅ₐ, or R₅ₐ and R₆ₐ are combined to form a heterocyclic ring optionally substituted with a photoreactive moiety; wherein at least two of R₂ₐ, R₃ₐ, R₄ₐ, R₅ₐ, and R₆ₐ are not hydrogen;
R₇ₐ, R₈ₐ, R₉ₐ, and R₁₀ₐ are each independently a photoreactive moiety, H, —OH, —NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl are optionally substituted from 1 to 3 times with substituents selected from the group consisting of, halogen, —OH, —NH₂, C₁-C₆alkyl, C₂-C₆ alkenyl, C₁-C₆ alkoxy, —SH, and C₁-C₆ thioalkyl, and wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; and
R₁₁ₐ, R₁₂ₐ, R₁₃ₐ, R₁₄ₐ, R₁₅ₐ, and R₁₆ₐ, are each independently a photoreactive moiety, H, halogen, —OH, —O—C₁-C₆ alkyl, —O—C₂-C₆ alkenyl, —O—C₂-C₆ alkynyl, —NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, wherein the alkyl, alkenyl, alkynyl, cycloalkyl, cycloalkylalkyl, arylalkyl, and mono or polycyclic aryl are optionally substituted with a photoreactive moiety and each one of Ruia-R₁₆, is optionally substituted with —NH₂, —OH, halogen, —COOH, —NO₂, or —CN;
IV) compounds of Formula ID: 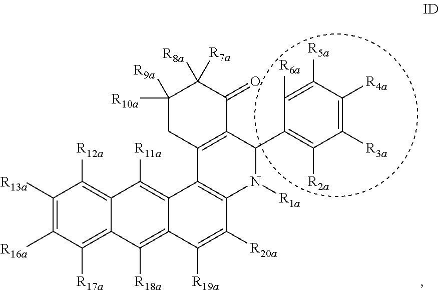
wherein:
the dotted circle identifies an active moiety;
R₁ₐ is H, —OH, —OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₂ₐ, R₃ₐ, R₄ₐ, R₅ₐ, and R₆ₐ are each independently a photoreactive moiety, H, halogen, —NO₂, —OH, —OR₁₄ₐ, —SR₁₄ₐ, —NH₂, —NHR₁₄ₐ, —NR₁₄ₐR₁₅ₐ, R₁₄ₐC(O)—, R₁₄ₐOC(O)—, R₁₄ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; or R₂ₐ, and R₃ₐ, R₃ₐ and R₄ₐ, R₄ₐ and R₅ₐ, or R₅ₐ and R₆ₐ are combined to form a heterocyclic ring optionally substituted with a photoreactive moiety; wherein at least two of R₂ₐ, R₃ₐ, R₄ₐ, R₅ₐ, and R₆ₐ are not hydrogen;
R₇ₐ, R₈ₐ, R₉ₐ, and R₁₀ₐ are each independently a photoreactive moiety, H, —OH, —NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl are optionally substituted from 1 to 3 times with substituents selected from the group consisting of, halogen, —OH, —NH₂, C₁-C₆alkyl, C₂-C₆ alkenyl, C₁-C₆ alkoxy, —SH, and C₁-C₆ thioalkyl, and wherein the alkyl, alkenyl, alkynyl, cycloalkyl, cycloalkylalkyl, arylalkyl, mono or polycyclic aryl, and mono or polycyclic heteroaryl are optionally substituted with a photoreactive moiety; and
R₁₁ₐ, R₁₂ₐ, R₁₃ₐ, R₁₄ₐ, R₁₅ₐ, R₁₆ₐ, R₁₇ₐ, R₁₈ₐ, R₁₉ₐ and R₂₀ₐ are each independently a photoreactive moiety, H, halogen, —OH, —O—C₁-C₆ alkyl, —O—C₂-C₆ alkenyl, —O—C₂-C₆ alkynyl, —NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, wherein the alkyl, alkenyl, alkynyl, cycloalkyl, cycloalkylalkyl, arylalkyl, and mono or polycyclic aryl are optionally substituted with a photoreactive moiety and each one of R₁₁ₐ-R₂₀ₐ is optionally substituted with —NH₂, —OH, halogen, —COOH, —NO₂, or —CN;
V) compounds of Formula II: 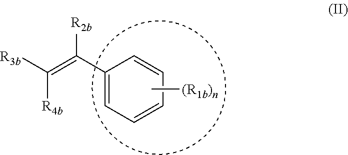
wherein:
the dotted circle identifies an active moiety;
n is an integer from 1 to 4;
R_{1b} is independently at each occurrence H, OH, OR_{5b}, halogen, CN, NO₂, NH₂, NHR_{5b}, NR_{5b}R_{6b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen;
R_{2b} is independently H, halogen, C₁-C₆ alkyl, C₂-C₆ alkenyl, C₂-C₆ alkynyl, C₃-C₆ cycloalkyl, C₄-C₇ cycloalkylalkyl, or mono or polycyclic aryl;
R_{3b} and R_{4b} are independently H, OR_{5b}, SR_{5b}, R_{5b}S(O)—, R_{5b}S(O)₂—, —COOR_{5b}, —C(O)NR_{5b}R_{6b}, C₁-C₆ alkyl, C₂-C₆ alkenyl, C₂-C₆ alkynyl, C₃-C₆ cycloalkyl, C₄-C₇cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; or
R_{3b} and R_{4b} can combine together to form a mono or polycyclic heterocyclyl or heteroaryl containing from 1-5 heteroatoms selected from the group consisting of nitrogen, sulfur, and oxygen, each formed heteroaryl or heterocyclyl optionally substituted with substituents selected from the group consisting of oxo, thio, amino, C₁-C₆ alkyl, C₂-C₆ alkenyl, and C₂-C₆ alkynyl; and
R_{5b} and R_{6b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, each one of R_{5b} or R_{6b} optionally substituted from 1 to 3 times with substituents selected from the group consisting of H, C₁-C₆ alkyl, C₂-C₆ alkenyl, C₂-C₆ alkynyl, C₃-C₆ cycloalkyl, and C₄-C₇ cycloalkylalkyl;
VI) compounds of Formula III: 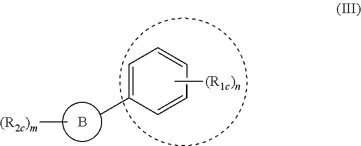
wherein:
the dotted circle identifies an active moiety;
m and n are integers from 1 to 4;
B is a substituted or unsubstituted mono or polycyclic aryl or mono or polycyclic heterocyclyl or heteroaryl with each cyclic unit containing from 1 to 5 heteroatoms selected from the group consisting of nitrogen, sulfur, and oxygen;
R_{1c} and R_{2c} are independently H, OH, OR_{3c}, halogen, CO, CN, NO₂, COOH, NH₂, NHR_{3c}, NR_{3c}R_{4c}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3c} and R_{4c} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen; and
VII) compounds of Formula IV 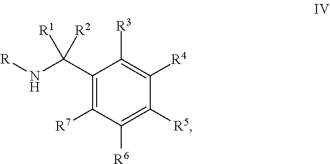
wherein:
R is selected from the group consisting of monocyclic or bicyclic aryl, monocyclic or bicyclic heteroaryl, and monocyclic or bicyclic heterocyclyl, wherein each monocyclic or bicyclic aryl, monocyclic or bicyclic heteroaryl, and monocyclic or bicyclic heterocyclyl can be optionally substituted from 1 to 4 times with substituents independently selected at each occurrence thereof from the group consisting of H, halogen, C₁₋₆ alkyl, aryl, —OR⁸, —CF₃, and —CHF₂;
R¹ and R² are each independently selected from the group consisting of a photoreactive moiety, H, halogen, and C₁₋₆ alkyl; or R¹ and R² are combined to form ═O;
R³, R⁴, R⁵, R⁶, and R⁷ are each independently selected from the group consisting of a photoreactive moiety, H, halogen, —NO₂, —NR⁸R⁹, —SO₂NR⁸R⁹, —N₃, —C(O)R⁸, aryl, heteroaryl, heterocyclyl, and 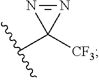 [SMILES: *C1(C(F)(F)F)N=N1]
and
R⁸ and R⁹ are each independently selected from the group consisting of H, C₁₋₆ alkyl, C₂₋₆ alkenyl, C₂₋₆ alkynyl, and aryl; or R⁸ and R⁹ are combined with the nitrogen to which they are attached to form a heterocyclyl, wherein the heterocyclyl can be optionally substituted with —COOH or —COOMe.

11-16. (canceled)

17. The method according to claim 10, wherein 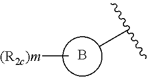
has the formula: 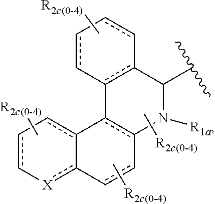
wherein
X is carbon or nitrogen;
R₁ₐ is H, OH, OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—;
R₁₄ₐ is H, halogen, OH, NO₂, C₁-C₆ alkyl, C₂-C₆ alkenyl, C₂-C₆ alkynyl, C₃-C₆ cycloalkyl, C₄-C₇cycloalkylalkyl, aryl C₁-C₆ alkyl, or mono or polycyclic aryl, with R₁ is optionally substituted with NH₂, OH, halogen, COOH, NO₂, or CN; and
the total number of R_{2c} substituents is from 1 to 4.

18. The method according to claim 11, wherein R₄ₐ is not hydrogen.

19. The method according to claim 11, wherein at least two of R₂ₐ, R₃ₐ, R₄ₐ, R₅ₐ, and R₆ₐ are not hydrogen.

20-21. (canceled)

22. The method according to claim 11, wherein R⁵ is not hydrogen.

23. The method according to claim 11, wherein at least two of R³, R⁴, R⁵, R⁶, and R⁷ are not hydrogen.

24. (canceled)

25. The method according to claim 10, wherein the compound comprises an active moiety having a formula selected from the group consisting of:

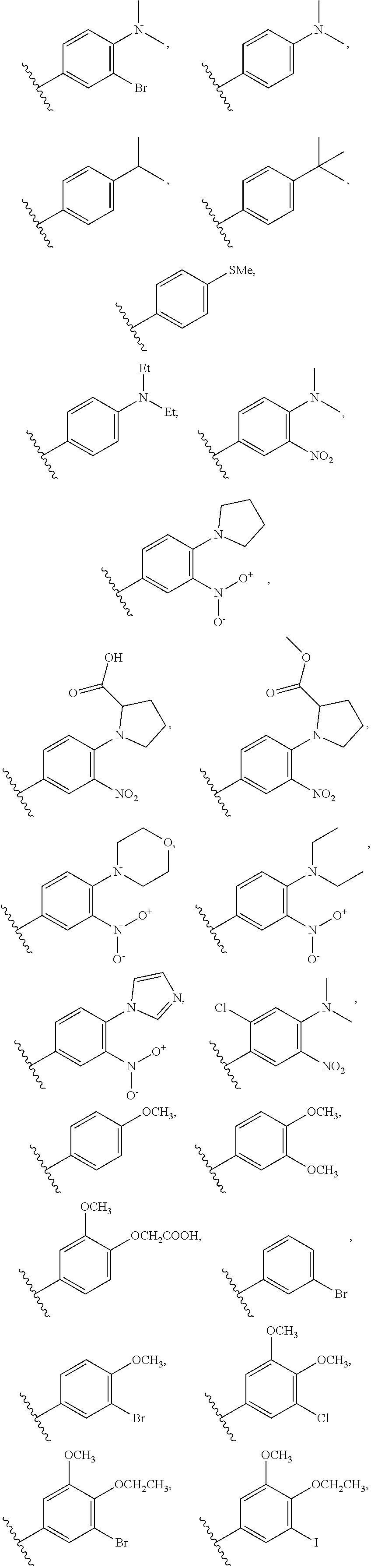

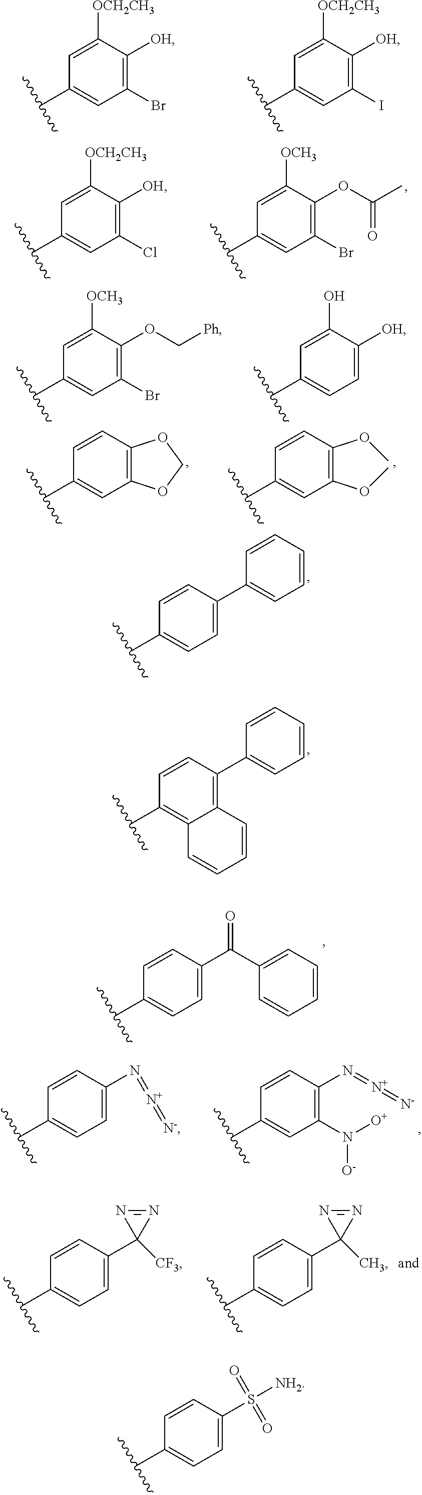

26-28. (canceled)

29. The method according to claim 2, wherein the condition is a cancer.

30. The method according to claim 1, wherein the cancer exhibits active GLS2 glutaminase activity.

31. The method according to claim 1, wherein the cancer is selected from the group consisting of breast cancer, triple-negative breast cancer, receptor-positive breast cancer, acute myeloid leukemia, bladder cancer, bladder urothelial carcinoma, brain lower grade glioma, cervical cancer, cervical squamous cell carcinoma, radiation-resistant cervical cancer, colorectal cancer, colorectal tumor, colon adenocarcinoma, glioblastoma multiforme, head and neck cancer, head and neck squamous cell carcinoma, kidney cancer, kidney chromophobe, kidney renal papillary cell carcinoma, large B cell lymphoma, liver cancer, liver hepatocellular carcinoma, lung cancer, lung adenocarcinoma, lung squamous cell carcinoma, melanoma, non-small cell lung cancer, neuroblastoma, ovarian cancer, ovarian serous cystadenocarcinoma, pancreatic cancer, pancreatic adenocarcinoma, pancreatic ductal adenocarcinoma, paraganglial cancer, paraganglioma, prostate cancer, prostate adenocarcinoma, rectal cancer, rectal adenocarcinoma, testicular cancer, testicular germ cell tumors, thymal cancer, thymoma, thyroid cancer, thyroid carcinoma, and uterine corpus endometrial carcinoma.

32-33. (canceled)

34. The method according to claim 1, wherein the cancer is characterized by moderate-to-high GLS2 expression and low GLS expression.

35-43. (canceled)

44. The method according to claim 1, wherein the cancer is resistant to treatment with a GLS-specific inhibitor.

45. (canceled)

46. The method according to claim 1, wherein said contacting comprises inhibiting cell proliferation, tumorigenesis, tumor growth, tumor initiation, and/or metastasis.

47. The method according to claim 2, wherein said administering is performed parenterally, orally, subcutaneously, intravenously, intramuscularly, extraperitoneally, by intranasal instillation, or by application to mucous membranes.

48-55. (canceled)
